(19) United States
(12) Patent Application Publication
(10) Pub. No.: US 2024/0050444 A1
(43) Pub. Date: Feb. 15, 2024

(54) TREATMENT METHODS

(71) Applicant: Levon Michael KHACHIGIAN, Randwick NSW (AU)

(72) Inventors: Levon Michael KHACHIGIAN, Randwick NSW (AU); Sebastian M. MARCUCCIO, Scoresby VIC (AU)

(21) Appl. No.: 17/906,288

(22) PCT Filed: Mar. 12, 2021
(86) PCT No.: PCT/AU2021/050219

§ 371 Date: Sep. 14, 2022

(30) Foreign Application Priority Data
Mar. 14, 2020 (AU) 2020900782

(51) Int. Cl. A61K 31/553; A61P 27/02; A61P 19/02

(52) U.S. Cl. CPC A61K 31/553; A61P 27/02; A61P 19/02

(57) ABSTRACT

A method of reducing vascular permeability, neovascularisation, angiogenesis, inflammation, cell migration and/or proliferation, comprising administering an effective amount of an inhibitor of FosB/ΔFosB expression and/or VCAM-1 expression and/or ERK1/2 phosphorylation, and pharmaceutical compositions and kits comprising inhibitors of FosB/ΔFosB expression and/or VCAM-1 expression and/or ERK1/2 phosphorylation.

### FIELD OF THE INVENTION

The present invention also relates to methods, compounds, and pharmaceutical compositions for reducing vascular permeability, neovascularisation, angiogenesis, inflammation, cell migration and/or cell proliferation, and to methods for inhibiting FosB/ΔFosB expression and/or ERK1/2 phosphorylation and/or VCAM-1 expression.

### BACKGROUND

Vascular permeability and neovascularization are key features underpinning inflammation, wound healing, tumor growth, macular edema in both diabetic retinopathy (DR) and neovascular (wet/exudative) age-related macular degeneration (nAMD). DR is the world's leading cause of vision loss in patients aged 20 to 74 years. AMD has a global prevalence of 170 million with around 11 million people affected with AMD in the United States. Retinal vascular leakage is caused by breakdown of the blood-retinal barrier (BRB) which normally maintains homeostasis. This is precipitated by endothelial dysfunction, angiogenic and inflammatory processes causing retinal capillary leakage into the interstitial space and edema through increased osmotic pressure. Vascular permeabilizing factors include vascular endothelial growth factor (VEGF), tumour necrosis factor-α (TNF-α), histamine, platelet-activating factor, serotonin and interleukin-β (IL-β).

Anti-VEGF therapies are widely used clinically for the treatment of DR. Repeated intravitreal injections, however, are needed and many patients do not respond optimally or an improved response is not sustained. Agents that target not only VEGF but other key mediators involved in the pathogenesis of nAMD/DR would have particular pharmaceutical appeal in this area of unmet clinical need.

Vascular permeability is also key to the pathogenesis of rheumatoid arthritis (RA), a process mediated by pro-inflammatory cytokines. RA impacts around 1.3 million people in the US alone.

There has been significant improvement in the management of RA over the last decade using biological agents, such as anti-TNF agents and soluble TNF receptor. However, a significant proportion of patients do not achieve clinical remission with current therapeutic options and are at risk of progressive joint destruction and functional disability.

Given the world's ageing population the significant unmet clinical need for both RA and nAMD/DR, and the global economic burden that the impact of these chronic diseases represent, alternative therapeutic approaches are needed.

### SUMMARY

Activator protein-1 (AP-1 or AP1) is a heterodimeric transcription factor involved in the regulation of gene expression in response to a range of pathological stimuli. The inventor has reasoned that compounds which are capable of inhibiting AP-1 dependent gene expression may be useful in treating or preventing diseases or conditions associated with vascular permeability, neovascularization, angiogenesis, inflammation, cell migration and/or cell proliferation.

The inventor has identified compounds that inhibit AP-1 dependent gene expression. The inventor has studied the activity of these compounds and found that these compounds inhibit FosB/ΔFosB expression. The inventor has found that such compounds are able to reduce vascular permeability, neovascularisation, angiogenesis, inflammation, cell migration and cell proliferation.

Accordingly, a first aspect provides a method of reducing vascular permeability, neovascularisation, angiogenesis, inflammation, cell migration and/or cell proliferation in a subject, comprising administering an effective amount of an inhibitor of FosB/ΔFosB expression.

An alternative first aspect provides an inhibitor of FosB/ΔFosB expression for use in reducing vascular permeability, neovascularisation, angiogenesis, inflammation, cell migration and/or cell proliferation in a subject; or use of an inhibitor of FosB/ΔFosB expression in the manufacture of a medicament for reducing vascular permeability, neovascularisation, angiogenesis, inflammation, cell migration and/or cell proliferation in a subject.

A second aspect provides a method of treating or preventing a disease or condition associated with vascular permeability, neovascularization, angiogenesis, inflammation, cell migration and/or cell proliferation in a subject, comprising administering to the subject an effective amount of an inhibitor of FosB/ΔFosB expression.

A alternative second aspect provides an inhibitor of FosB/ΔFosB expression for use in treating or preventing a disease or condition associated with vascular permeability, neovascularisation, angiogenesis, inflammation, cell migration and/or cell proliferation in a subject; or use of an inhibitor of FosB/ΔFosB expression in the manufacture of a medicament for treating or preventing a disease or condition associated with vascular permeability, neovascularisation, angiogenesis, inflammation, cell migration and/or cell proliferation in a subject.

A third aspect provides a method of reducing vascular permeability, neovascularization, angiogenesis, inflammation, cell migration and/or cell proliferation in a subject, comprising administering an effective amount of an inhibitor of FosB/ΔFosB expression, and/or extracellular signal-regulated kinase-1/2 (ERK1/2) phosphorylation and/or vascular cell adhesion molecule-1 (VCAM-1 or VCAM1) expression.

An alternative third aspect provides an inhibitor of FosB/ΔFosB expression, and/or ERK1/2 phosphorylation and/or VCAM-1 expression for use in reducing vascular permeability, neovascularization, angiogenesis, inflammation, cell migration and/or cell proliferation in a subject; or use of an inhibitor of FosB/ΔFosB expression, and/or ERK1/2 phosphorylation and/or VCAM-1 expression in the manufacture of a medicament for reducing vascular permeability, neovascularization, angiogenesis, inflammation, cell migration and/or cell proliferation in a subject.

A fourth aspect provides method of treating or preventing a disease or condition associated with vascular permeability, neovascularization, angiogenesis, inflammation, cell migration and/or cell proliferation in a subject, comprising administering an effective amount of an inhibitor of ERK1/2 phosphorylation, and/or FosB/ΔFosB expression, and/or VCAM-1 expression.

An alternative fourth aspect provides an inhibitor of FosB/ΔFosB expression, and/or ERK1/2 phosphorylation and/or VCAM-1 expression for use in treating or preventing a disease or condition associated with vascular permeability, neovascularization, angiogenesis, inflammation, cell migration and/or cell proliferation in a subject; or use of an inhibitor of FosB/ΔFosB expression, and/or ERK1/2 phosphorylation and/or VCAM-1 expression in the manufacture of a medicament for treating or preventing a disease or condition associated with vascular permeability, neovascularization, angiogenesis, inflammation, cell migration and/or cell proliferation in a subject.

A fifth aspect provides a method of reducing vascular permeability, neovascularization, angiogenesis, inflammation, cell migration and/or cell proliferation in a subject, comprising administering an effective amount of a compound of formula I or II, or a pharmaceutically acceptable salt thereof:

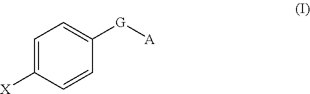

wherein:

X is F, Cl, Br or I;

G is C═O or C═N—OH; and

A is:

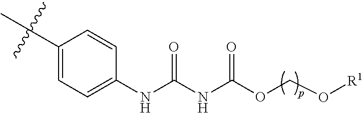

wherein p is 1, 2, 3 or 4; and R¹ is straight or branched C₁-C₆ alkyl;

or A is:

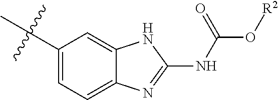

wherein R² is straight or branched C₁-C₆ alkyl;

or

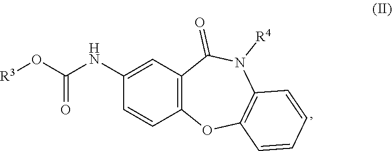

wherein:

R³ is straight or branched C₁-C₆ alkyl; and

R⁴ is straight or branched C₁-C₆ alkyl,

or R⁴ is

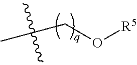

wherein q is 1, 2, 3 or 4; and R⁵ is straight or branched C₁-C₆ alkyl.

An alternative fifth aspect provides a compound of formula I or II, or a pharmaceutically acceptable salt thereof, for use in reducing vascular permeability, neovascularization, angiogenesis, inflammation, cell migration and/or cell proliferation in a subject; or use of a compound of formula I or II, or a pharmaceutically acceptable salt thereof, in the manufacture of a medicament for reducing vascular permeability, neovascularization, angiogenesis, inflammation, cell migration and/or cell proliferation in a subject.

A sixth aspect provides a method of treating or preventing a disease or condition mediated by AP-1 and/or ERK1//2, comprising administering to the subject an effective amount of a compound of formula I or II, or a pharmaceutically acceptable salt thereof:

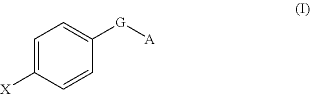

wherein:

X is F, Cl, Br or I;

G is C═O or C═N—OH; and

A is:

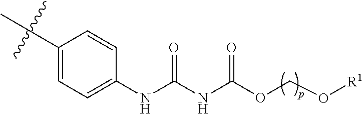

wherein p is 1, 2, 3 or 4; and R¹ is straight or branched C₁-C₆ alkyl;

or A is:

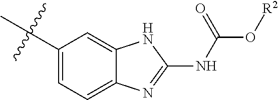

wherein R² is straight or branched C₁-C₆ alkyl;

or

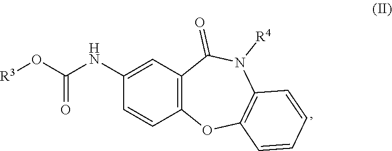

wherein:

R³ is straight or branched C₁-C₆ alkyl; and

R⁴ is straight or branched C₁-C₆ alkyl,

or R⁴ is

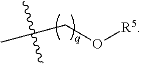

An alternative sixth aspect provides a compound of formula I or II, or a pharmaceutically acceptable salt thereof, for use in treating or preventing a disease or condition mediated by AP-1, and/or ERK1/2, in a subject; or use of a compound of formula I or II, or a pharmaceutically acceptable salt thereof, in the manufacture of a medicament for treating or preventing a disease or condition mediated by AP-1, and/or ERK1/2, in a subject.

A seventh aspect provides a method of treating or preventing a disease or condition mediated by AP-1, and/or FosB/ΔFosB and/or ERK1/2 and/or VCAM-1 and/or VEGF-A and/or IL-1β, in a subject, comprising administering to the subject an effective amount of a compound of formula I or II, or a pharmaceutically acceptable salt thereof:

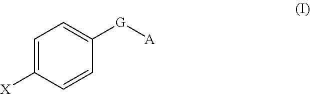

wherein:

X is F, Cl, Br or I;

G is C═O or C═N—OH; and

A is:

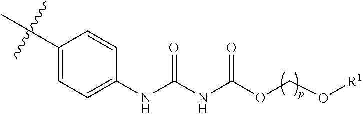

wherein p is 1, 2, 3 or 4; and R¹ is straight or branched C₁-C₆ alkyl;

or A is:

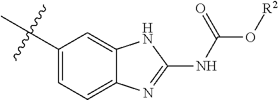

wherein R² is straight or branched C₁-C₆ alkyl;

or

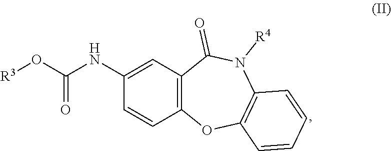

wherein:

R³ is straight or branched C₁-C₆ alkyl; and

R⁴ is straight or branched C₁-C₆ alkyl,

or R⁴ is

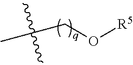

wherein q is 1, 2, 3 or 4; and R⁵ is straight or branched C₁-C₆ alkyl.

An alternative seventh aspect provides a compound of formula I or II, or a pharmaceutically acceptable salt thereof, for use in treating or preventing a disease or condition mediated by AP-1, and/or FosB/ΔFosB and/or ERK1/2 and/or VCAM-1 and/or VEGF-A and/or IL-1β, in a subject; or use of a compound of formula I or II, or a pharmaceutically acceptable salt thereof, in the manufacture of a medicament for treating or preventing a disease or condition mediated by AP-1, and/or FosB/ΔFosB and/or ERK1/2 and/or VCAM-1 and/or VEGF-A and/or IL-1β, in a subject.

An eighth aspect provides a method of treating or preventing a disease or condition associated with vascular permeability, neovascularization, angiogenesis, inflammation, cell migration and/or cell proliferation in a subject, comprising administering to the subject an effective amount of a compound of formula I or II, or a pharmaceutically acceptable salt thereof:

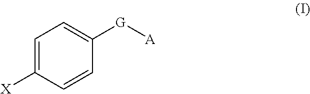

wherein:

X is F, Cl, Br or I;

G is C═O or C═N—OH; and

A is:

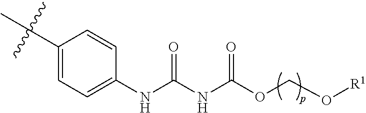

wherein p is 1, 2, 3 or 4; and R¹ is straight or branched C₁-C₆ alkyl;

or A is:

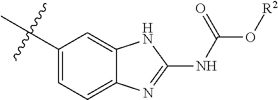

wherein R² is straight or branched C₁-C₆ alkyl;

or

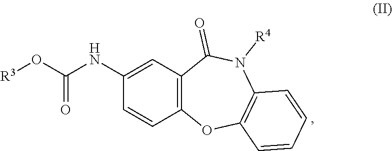

wherein:

R³ is straight or branched C₁-C₆ alkyl; and

R⁴ is straight or branched C₁-C₆ alkyl,

or R⁴ is

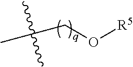

wherein q is 1, 2, 3 or 4; and R⁵ is straight or branched C₁-C₆ alkyl.

An alternative eighth aspect provides a compound of formula I or II, or a pharmaceutically acceptable salt thereof, for use in treating or preventing a disease or condition associated with vascular permeability, neovascularization, angiogenesis, inflammation, cell migration and/or cell proliferation in a subject; or use of a compound of formula I or II, or a pharmaceutically acceptable salt thereof, in the manufacture of a medicament for treating or preventing a disease or condition associated with vascular permeability, neovascularization, angiogenesis, inflammation, cell migration and/or cell proliferation in a subject.

A ninth aspect provides a method of reducing vascular permeability, neovascularization, angiogenesis, inflammation, cell migration and/or cell proliferation in a subject, comprising administering to the subject an effective amount of a compound selected from:

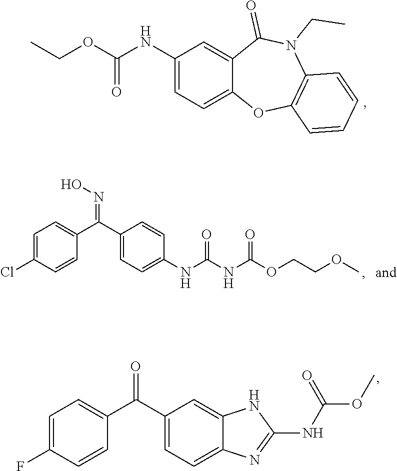 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

An alternative ninth aspect provides a compound selected from:

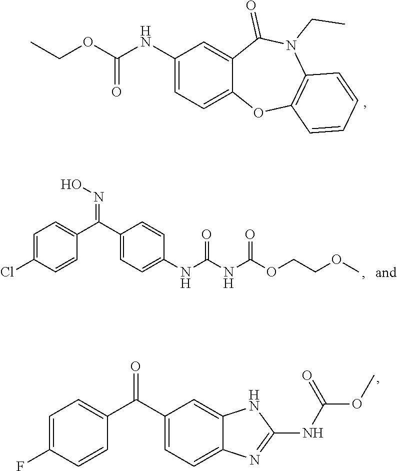 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in reducing vascular permeability, neovascularization, angiogenesis, inflammation, cell migration and/or cell proliferation in a subject; or use of a compound selected from:

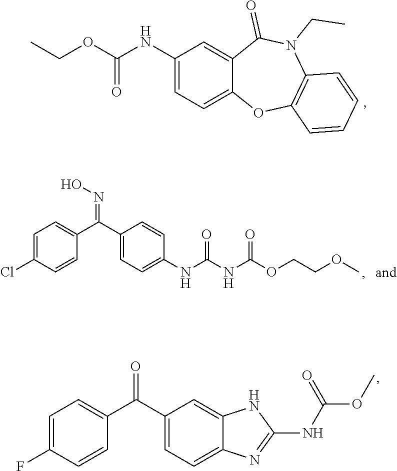 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, in the manufacture of a medicament for reducing vascular permeability, neovascularization, angiogenesis, inflammation, cell migration and/or cell proliferation in a subject

A tenth aspect provides a method of reducing AP-1-dependent gene expression and/or ERK1/2-dependent gene expression in a subject, comprising administering to the subject an effective amount of a compound selected from:

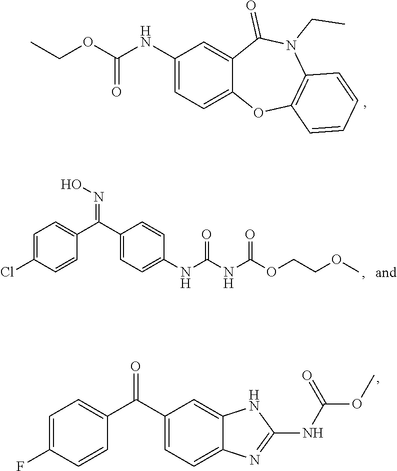 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

An alternative tenth aspect provides a compound selected from:

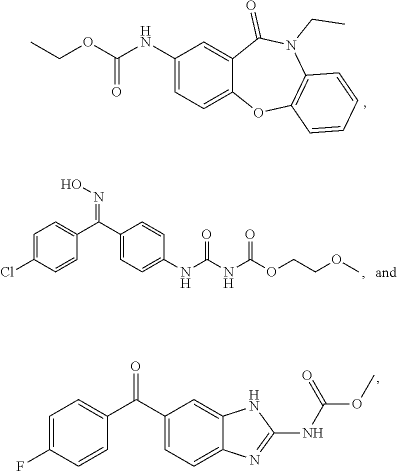 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in reducing AP-1-dependent gene expression and/or ERK1/2-dependent gene expression in a subject; or use of a compound selected from:

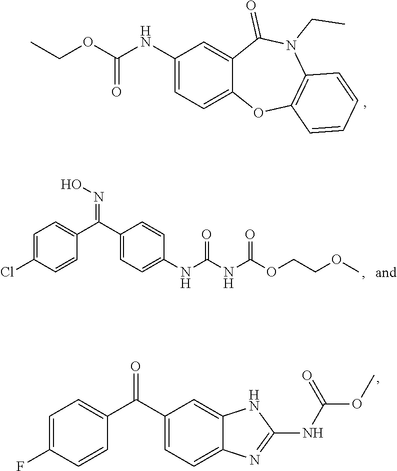 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, in the manufacture of a medicament for reducing AP-1-dependent gene expression and/or ERK1/2-dependent gene expression in a subject

An eleventh aspect provides a method of treating or preventing a disease or condition mediated by AP-1 and/or FosB/ΔFosB, and/or ERK1/2 and/or VCAM-1, and/or VEGF-A, and/or IL-1β in a subject, comprising administering to the subject an effective amount of a compound selected from:

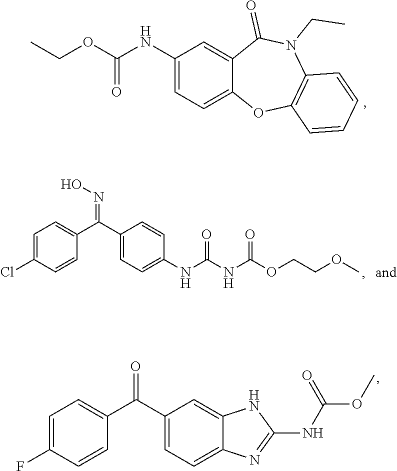 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

An alternative eleventh aspect provides a compound selected from:

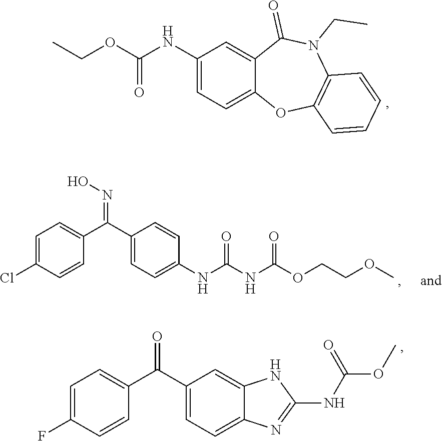 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in treating or preventing a disease or condition mediated by AP-1 and/or FosB/ΔFosB, and/or ERK1/2 and/or VCAM-1, and/or VEGF-A, and/or IL-1β in a subject; or use of a compound selected from:

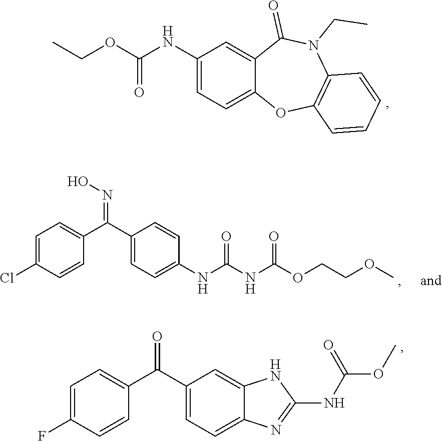 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, in the manufacture of a medicament for treating or preventing a disease or condition mediated by AP-1 and/or FosB/ΔFosB, and/or ERK1/2 and/or VCAM-1, and/or VEGF-A, and/or IL-1β in a subject.

A twelfth aspect provides a method of treating or preventing a condition associated with vascular permeability, neovascularization, angiogenesis, inflammation, cell migration and/or cell proliferation in a subject, comprising administering to the subject an effective amount of a compound selected from:

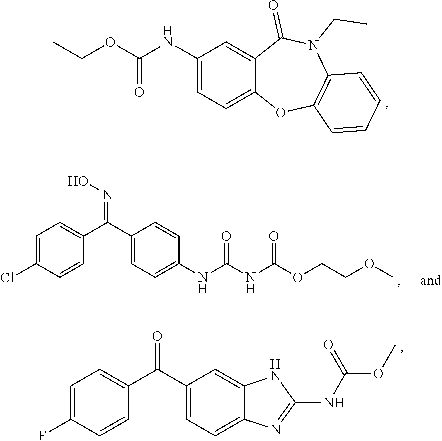 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

An alternative twelfth aspect provides a compound selected from:

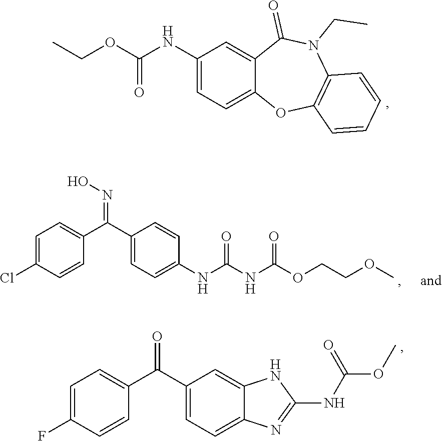 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in treating or preventing a condition associated with vascular permeability, neovascularization, angiogenesis, inflammation, cell migration and/or cell proliferation in a subject; or use of a compound selected from:

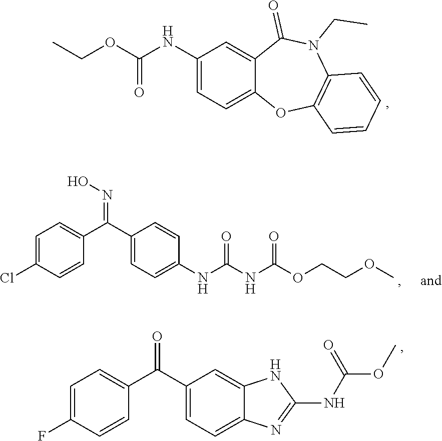 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, in the manufacture of a medicament for treating or preventing a condition associated with vascular permeability, neovascularization, angiogenesis, inflammation, cell migration and/or cell proliferation in a subject.

A thirteenth aspect provides a method of reducing ERK1/2 phosphorylation, and/or FosB/ΔFosB expression, and/or VCAM-1 expression and/or VEGF-A expression in a cell, comprising contacting the cell with an effective amount of a compound of formula I or II, or a pharmaceutically acceptable salt thereof:

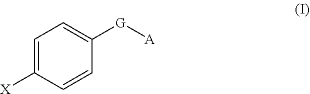

wherein:

X is F, Cl, Br or I;

G is C═O or C═N—OH; and

A is:

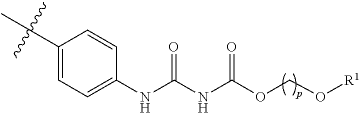

wherein p is 1, 2, 3 or 4; and R¹ is straight or branched C₁-C₆ alkyl;

or A is:

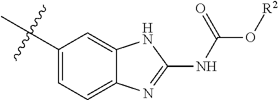

wherein R² is straight or branched C₁-C₆ alkyl;

or

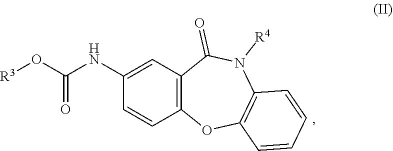

wherein:

R³ is straight or branched C₁-C₆ alkyl; and

R⁴ is straight or branched C₁-C₆ alkyl,

or R⁴ is

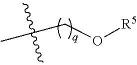

wherein q is 1, 2, 3 or 4; and R⁵ is straight or branched C₁-C₆ alkyl.

A fourteenth aspect provides a method of reducing ERK1/2 phosphorylation, and/or FosB/ΔFosB expression and/or VCAM-1 expression and/or VEGF-A expression in a cell, comprising contacting the cell with an effective amount of a compound selected from:

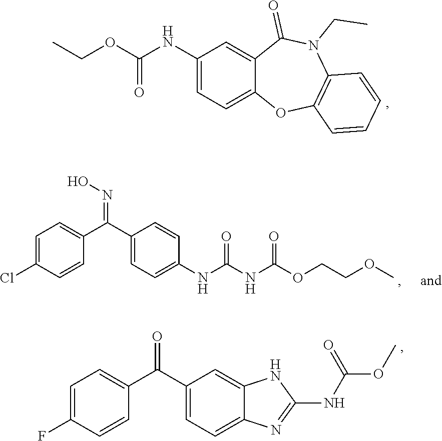 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

A fifteenth aspect provides a method of inhibiting ERK1/2 phosphorylation, comprising incubating ERK1/2 with an effective amount of a compound of formula I or II, or a pharmaceutically acceptable salt thereof:

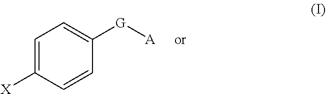

wherein:

X is F, Cl, Br or I;

G is C═O or C═N—OH; and

A is:

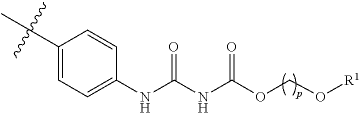

wherein p is 1, 2, 3 or 4; and R¹ is straight or branched C₁-C₆ alkyl;

or A is:

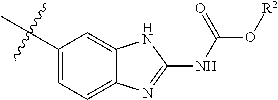

wherein R² is straight or branched C₁-C₆ alkyl;

or

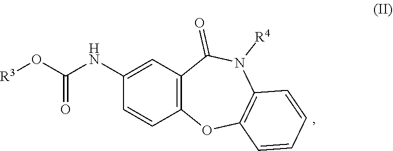

wherein:

R³ is straight or branched C₁-C₆ alkyl; and

R⁴ is straight or branched C₁-C₆ alkyl,

or R⁴ is

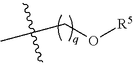

wherein q is 1, 2, 3 or 4; and R⁵ is straight or branched C₁-C₆ alkyl.

A sixteenth aspect provides a method of inhibiting ERK1/2 phosphorylation, comprising incubating ERK1/2 with an effective amount of a compound selected from:

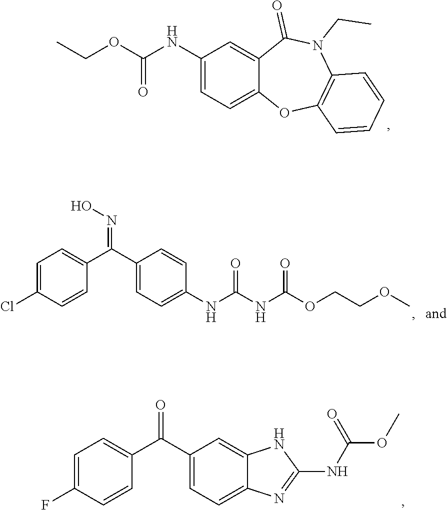 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

A seventeenth aspect provides a pharmaceutical composition comprising a compound which is an inhibitor of FosB/ΔFosB expression, and optionally an inhibitor of ERK1/2 phosphorylation and/or VCAM-1 expression, and a pharmaceutically acceptable carrier.

An eighteenth aspect provides a pharmaceutical composition comprising a compound of the following formula:

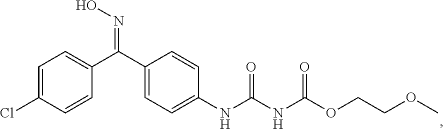 [SMILES: COCCOC(=O)NC(=O)Nc1ccc(/C(=N/O)c2ccc(Cl)cc2)cc1]

or a pharmaceutically acceptable salt thereof, and a pharmaceutically acceptable carrier.

A nineteenth aspect provides a method of treating or preventing a disease or condition selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject, comprising administering an effective amount of an inhibitor of FosB/ΔFosB expression; and optionally an inhibitor of ERK1/2 phosphorylation and/or VCAM-1 expression.

An alternative nineteenth aspect provides an inhibitor of FosB/ΔFosB expression; and optionally an inhibitor of ERK1/2 phosphorylation and/or VCAM-1 expression for use in treating or preventing a disease or condition selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject; or use of an inhibitor of FosB/ΔFosB expression; and optionally an inhibitor of ERK1/2 phosphorylation and/or VCAM-1 expression in the manufacture of a medicament for treating or preventing a disease or condition selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
restenosis;
inflammation;
cytokine storm;
pulmonary inflammation;
pulmonary fibrosis
   in a subject.

A twentieth aspect provides a method of treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject, comprising administering an effective amount of a compound of formula I or II, or a pharmaceutically acceptable salt thereof.

An alternative twentieth aspect provides a compound of formula I or II, or a pharmaceutically acceptable salt thereof, for use in treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject; or use of a compound of formula I or II, or a pharmaceutically acceptable salt thereof, in the manufacture of a medicament for treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject.

A twenty first aspect provides a method of treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
restenosis;
inflammation;
cytokine storm;
pulmonary inflammation;
pulmonary fibrosis
   in a subject, comprising administering an effective amount of a compound selected from:

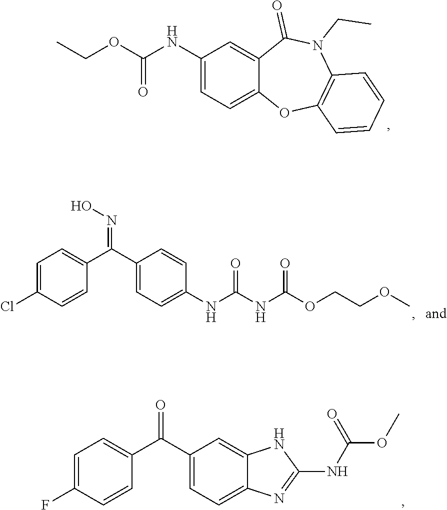 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

A twentieth aspect provides a compound having the following formula:

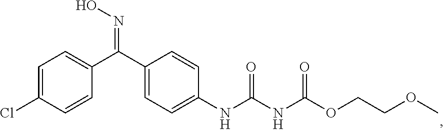 [SMILES: COCCOC(=O)NC(=O)Nc1ccc(/C(=N/O)c2ccc(Cl)cc2)cc1]

or a pharmaceutically acceptable salt thereof.

An alternative twenty first aspect provides a compound selected from:

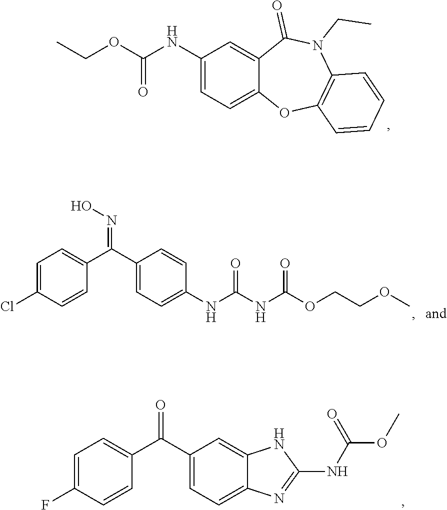 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject; or use of a compound selected from:

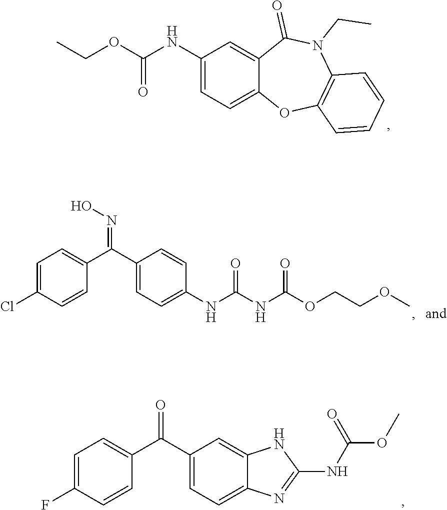 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, in the manufacture of a medicament for treating or preventing a condition or disease selected from:
arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation;
pulmonary fibrosis
   in a subject.

A twenty second aspect provides a pharmaceutical composition comprising a compound of formula II, or a pharmaceutically acceptable salt thereof, and a pharmaceutically acceptable carrier.

A twenty third aspect provides a pharmaceutical composition comprising a compound of the following formula:

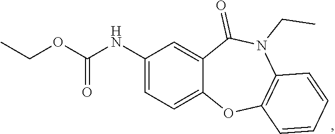 [SMILES: CCOC(=O)Nc1ccc2c(c1)C(=O)N(CC)c1ccccc1O2]

or a pharmaceutically acceptable salt thereof, and a pharmaceutically acceptable carrier.

A twenty fourth aspect provides use of a compound of formula I or II, or a pharmaceutically acceptable salt thereof, for reducing ERK1/2 phosphorylation, and/or FosB/ΔFosB expression, and/or VCAM-1 expression, and/or VEGF-A expression, in vitro.

A twenty fifth aspect provides a compound of formula I or II, or a pharmaceutically acceptable salt thereof, for use in reducing ERK1/2 phosphorylation, and/or FosB/ΔFosB expression, and/or VCAM-1 expression, and/or VEGF-A expression, in vitro.

A twenty sixth aspect provides a method of reducing ERK1/2 phosphorylation, and/or FosB/ΔFosB expression and/or VCAM-1 expression and/or VEGF-A expression in a cell in vitro, comprising contacting the cell with an effective amount of a compound of formula I or II, or a pharmaceutically acceptable salt thereof.

A twenty seventh aspect provides use of a compound selected from the following formula

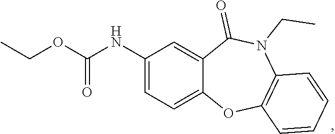 [SMILES: CCOC(=O)Nc1ccc2c(c1)C(=O)N(CC)c1ccccc1O2]

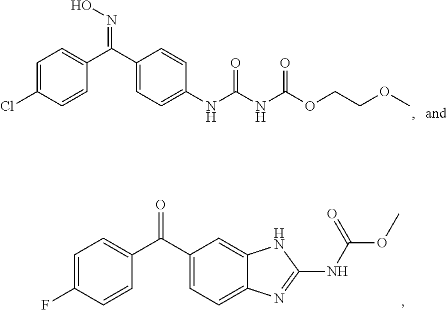 [SMILES: COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for reducing ERK1/2 phosphorylation, and/or FosB/ΔFosB expression, and/or VCAM-1 expression, and/or VEGF-A expression, in vitro.

A twenty eighth aspect provides a compound selected from the following formula

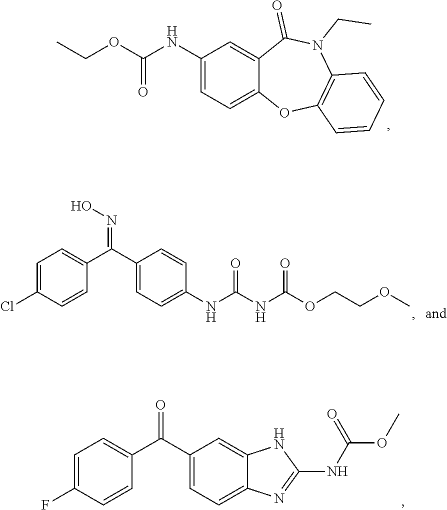 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof, for use in reducing ERK1/2 phosphorylation, and/or FosB/ΔFosB expression, and/or VCAM-1 expression, and/or VEGF-A expression, in vitro.

A twenty ninth aspect provides a method of reducing ERK1/2 phosphorylation, and/or FosB/ΔFosB expression and/or VCAM-1 expression and/or VEGF-A expression in a cell in vitro, comprising contacting the cell with an effective amount of a compound selected from the following formula

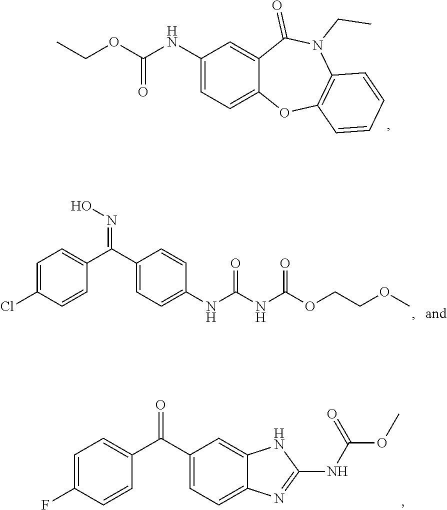 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

A thirtieth aspect provides a method of reducing expression of a gene referred to in Table 3A, 3B and/or 3C, typically a gene induced by IL-1β referred to in Table 3A, 3B and/or 3C, more typically a gene induced by IL-1β and referred to Table 3B, comprising administering an effective amount of a compound of formula I or II, or a pharmaceutically acceptable salt thereof:

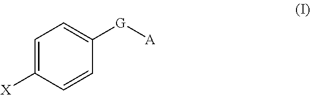

wherein:

X is F, Cl, Br or I;

G is C═O or C═N—OH; and

A is:

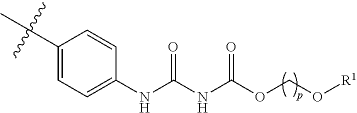

wherein p is 1, 2, 3 or 4; and R¹ is straight or branched C₁-C₆ alkyl;

or A is:

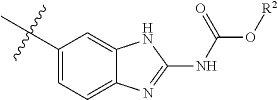

wherein R² is straight or branched C₁-C₆ alkyl;

or

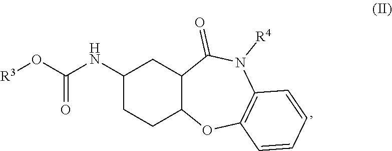

wherein:

R³ is straight or branched C₁-C₆ alkyl; and

R⁴ is straight or branched C₁-C₆ alkyl,

or R⁴ is

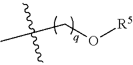

wherein q is 1, 2, 3 or 4; and R⁵ is straight or branched C₁-C₆ alkyl.

A thirty first aspect provides a method of treating or preventing a condition mediated by expression of a gene referred to in Table 3A, 3B and/or 3C, typically a gene induced by IL-1β and referred to in Table 3A, 3B and/or 3C, more typically a gene induced by IL-1β and referred to Table 3B, in a subject, comprising administering an effective amount of a compound of formula I or II, or a pharmaceutically acceptable salt thereof:

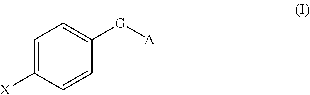

wherein:

X is F, Cl, Br or I;

G is C═O or C═N—OH; and

A is:

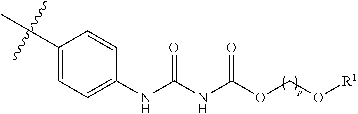

wherein p is 1, 2, 3 or 4; and R¹ is straight or branched C₁-C₆ alkyl;

or A is:

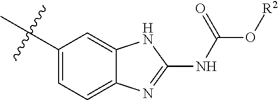

wherein R² is straight or branched C₁-C₆ alkyl;

or

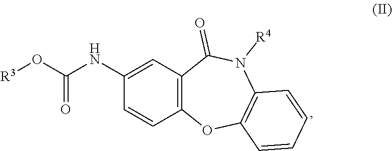

wherein:

R³ is straight or branched C₁-C₆ alkyl; and

R⁴ is straight or branched C₁-C₆ alkyl,

or R⁴ is

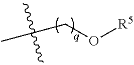

wherein q is 1, 2, 3 or 4; and R⁵ is straight or branched C₁-C₆ alkyl.

An alternative thirty first aspect provides a compound of formula I or II, or a pharmaceutically acceptable salt thereof for use in treating or preventing a condition mediated by expression of a gene referred to in Table 3A, 3B and/or 3C, typically a gene induced by IL-1β and referred to in Table 3A, 3B or 3C, more typically a gene induced by IL-1β and referred to Table 3B, in a subject; or use of a compound of formula I or II, or a pharmaceutically acceptable salt thereof in the manufacture of a medicament for treating or preventing a condition mediated by expression of a gene referred to in Table 3A, 3B and/or 3C, typically a gene induced by IL-1β referred to in Table 3A, 3B or 3C, more typically a gene induced by IL-1β and referred to Table 3B.

A thirty second aspect provides a method of reducing ICAM-1, c-Fos, Egr-1, CXCL2, KLF5, and/or VCAM-1 expression in a cell, comprising contacting the cell with a compound of formula I or II, or a pharmaceutically acceptable salt thereof.

A thirty third aspect provides a method of reducing expression of a gene referred to in Table 3A, 3B and/or 3C, typically a gene induced by IL-1β referred to in Table 3A, 3B or 3C, more typically a gene induced by IL-1β and referred to Table 3B, in a cell, comprising contacting the cell with an effective amount of a compound of formula I or II, or a pharmaceutically acceptable salt thereof.

### BRIEF DESCRIPTION OF THE FIGURES

Preferred embodiments of the present invention will now be described, by way of example only, with reference to the accompanying drawings:

FIG. 1A are images of Western blots showing the effect of compounds BT2, T4 and T6 on FosB/ΔFosB and c-Fos expression. HMEC-1 were grown in 6-well plates (in 10% FBS with EGF and hydrocortisone) and serum-arrested for 20 h, then treated with 30 μM compound (T4, T6, T7, BT2 and BT3) in serum free medium (without EGF or hydrocortisone) at 37° C. for 4 h. The medium was changed to 10% FBS (with EGF and hydrocortisone) with compound at the same concentration for 1 h. Lysates were resolved by SDS-PAGE and Western blotting was performed for FosB or c-Fos. Experiments were performed with independent biological duplicates where indicated. Approximate positions of molecular weight markers are shown. Data represent 3 biologically-independent experiments.

FIG. 1B shows the effect of BT2, T4 and T6 on serum-inducible endothelial cell proliferation over time. Serum-deprived HMEC-1 were treated with compound in medium containing 5% FBS (with EGF and hydrocortisone) and cell proliferation monitored using the xCELLigence system. Upper, Representative real time profiles from one experiment using the xCELLigence system with concentrations indicated. Cell index is a quantitative measure of cell growth. Lower, xCELLigence data represents the mean±SEM of the means of 5-8 independent experiments after 79 h. Statistical significance was assessed by one-way ANOVA.

FIG. 1C shows the effect of BT2, T4 and T6 on endothelial migration. BAEC in DMEM containing 10% FBS were seeded into 24-well plates fitted with 0.8 μm Transwell inserts. After 48 h, the medium was changed to DMEM containing 0.01% FBS for 48 h. Compounds were added to the upper chamber at 1 μM in DMEM containing 0.01% FBS and the medium in the lower chamber was changed to DMEM containing 10% FBS and 50 ng/ml VEGF-A₁₆₅. The cells were left for 24 h. Nuclei were quantified using NIH ImageJ software. Data represents the mean±SEM of the means of 4-5 independent experiments. Statistical significance was assessed by Kruskal-Wallis multiple comparisons test.

FIG. 1D shows the effect of BT2, T4 and T6 on endothelial cell regrowth after mechanical injury in vitro using a scratch assay. HMEC-1 monolayers scraped with a sterile toothpick were treated with compound at 0.6 μM in medium containing 5% FBS. Regrowth in the denuded area was monitored 48 h after scraping. Regrown area was determined using Image-Pro Plus software (Cybernetics). Data represents the mean±SEM of the means of 5 independent experiments. Statistical significance was assessed by one-way ANOVA.

FIG. 1E shows the effect of BT2, T4 and T6 on endothelial network (tubule) formation on Matrigel. HMEC-1 in medium containing 1% FBS and 50 ng/ml FGF-2 were mixed with compound (3 μM final) and seeded in wells coated with Matrigel. Network formation was assessed over the course of 24 h. Networks were quantified using Image-Pro Plus software. Data represents the mean±SEM of the means of 5-6 independent experiments. Statistical significance was assessed by Kruskal-Wallis multiple comparisons test.

FIG. 2A shows that BT2 inhibits retinal permeability in rats following choroidal laser injury. BT2, T4, T6 (doses indicated) or vehicle (control) was injected IVT into both eyes on the day (Day 0) and 7 days following 6 laser burns of the retina. Kenacort was administered IVT on Day 0. Alternatively, aflibercept/Eylea in vehicle (saline) was injected IVT 6 times (Days 0, 3, 7, 10, 14, 17). On Days 14 and 21, sodium fluorescein was injected subcutaneously and after 10 min, ocular fluorescence was recorded using Heidelberg retinal angiography (HRA) and scored. HRA score combines Day 14 and 21 data. Data represents mean±SEM. Statistical significance was assessed using one-way ANOVA (plot at left) or t-test (plot at right, and BT2 versus Kenacort comparison in plot at left). n=5-29 per group.

FIG. 2B shows that BT2 inhibits retinal vascular permeability in rabbits induced by rhVEGF-A₁₆₅. BT2 or BT3 (600 μg) or vehicle was injected IVT into the right eyes of rabbits 5d prior to induction of vascular leakage by IVT injection of 500 ng rhVEGF-A₁₆₅ in 50 μl in the same eyes. Two days after induction, sodium fluorescein was injected intravenously and after 1 h, ocular fluorescence was measured in right (R) and left (L) eyes with an ocular fluorophotometer and expressed as a ratio (R/L) for each rabbit. Ratio data from the vehicle and BT3 groups were pooled (control) as both conditions were inactive and did not differ statistically from the other, for comparison against active compound BT2. Data represents mean±SEM. Statistical significance was assessed by t-test. n=6-8 per group.

FIGS. 2C-E show immunohistochemical staining in rat retinal lesions for (C) CD31, (D) VEGF-A₁₆₅, (E) VEGF-A₁₆₅ in 100 μm boxed increments relative to the wound. Untreated refers to eyes that were not lasered or injected with vehicle or drug. IOD of positive staining (red chromogen) was assessed using Image-Pro Plus software. Slides were photographed under 10× or 20× objective and magnified views are shown. n=4-6 per group for CD31, n=3-6 per group for VEGF-A₁₆₅ or n=3-5 per group for VEGF-A₁₆₅ gradient analysis. Data represents the mean±SEM of the mean per animal. Statistical significance was assessed by one-way ANOVA, Mann-Whitney or t-test, as appropriate. Arrows provide examples of positive staining.

FIG. 2F shows that BT2 inhibits angiogenesis in Matrigel plugs in mice. Matrigel (500 μl) containing VEGF-A₁₆₅ (100 ng/ml), heparin (10 U) and BT2 or BT3 (2.5 mg/mouse) or vehicle was injected subcutaneously into the left flanks of male 8 week-old C57BL/6 mice. After 7 days, mice were sacrificed and the plugs stained with CD31 antibodies. Representative immunohistochemical images stained for CD31 photographed under 10× objective with the inset providing a magnified view (photographed under 40× objective). CD31 staining was quantified using Image-Pro Plus software. Data represents mean±SEM of the mean per animal. Statistical significance was assessed by Kruskal-Wallis multiple comparisons test. n=10-11 per group.

FIG. 3A are images of Western blots showing that BT2 inhibits ERK phosphorylation, FosB/ΔFosB and VCAM-1 expression. HMEC treated with 30 μM BT2 or 30 μM PD98059 were stimulated with 20 ng/ml IL-1β for various times up to 4 h. Westerns are representative of 2-3 biologically independent experiments each performed with 2 biologically independent replicates run in separate lanes (where shown) with times shown in hours. BT2 inhibition of IL-1β-inducible VCAM-1 and ERK phosphorylation on the same blot is indicated in FIG. 3D.

FIG. 3B shows that BT2 inhibits VCAM-1 expression by flow cytometry. Flow cytometry was performed with HMEC-1 treated with 30 μM BT2 or BT3 and 20 ng/ml IL-1β using a BD FACSCanto II. Data represents mean±SEM of the means of 3 independent experiments. Statistical significance was assessed by one-way ANOVA. n=3 per group.

FIG. 3C shows that BT2 inhibits FosB, c-Fos, VCAM-1, ICAM-1 and a range of other genes involved in cell proliferation, migration, angiogenesis and/or inflammation, RNA-seq was performed with total RNA prepared from HMEC-1 pre-treated with 30 μM BT2 and 4 h incubation with 20 ng/ml IL-β. A PCA plot (upper left) shows close association between biological replicates within conditions UT, IL-1β and IL-β+BT2 and clear separation across conditions. The heatmap (centre, 1579 genes) was generated for all up-regulated genes for the comparison IL-1β versus UT. Counts per million (cpm) values were used and the genes (rows) were grouped using hierarchical clustering with cpm for FosB and VCAM-1 and plotted. The heatmap (right) shows 325 genes with log fold change (FC) 2. FosB, c-Fos and VCAM-1 (the subject of this work) are indicated in the figure together with several other genes inhibited by BT2. The figure also shows a small subset of genes (indicated in red) that are further induced by BT2. BHLHE40, basic helix-loop-helix family member e40; CCL20, C-C motif chemokine ligand 20; CXCL2, C-X-C motif chemokine ligand 2; DUSP1, dual specificity phosphatase 1; EGR1, early growth response 1; ETS1, ETS proto-oncogene 1; FOS, FOS proto-oncogene; FOSB, FosB proto-oncogene; ICAM1, intercellular adhesion molecule 1; IL6, interleukin 6; KLF5, Kruppel like factor 5; MMP25, matrix metallopeptidase 25; NFKBIA, NFKB inhibitor α; THBS1, thrombospondin 1; TNIP, TNFAIP3 interacting protein 1; PLAT, plasminogen activator, tissue type; VCAM1, vascular cell adhesion molecule 1.

FIG. 3D shows that BT2 inhibits IL-β-inducible VCAM-1 expression and ERK phosphorylation more potently than PD98059. Concentrations of BT2 and PD98059 (1-30 μM) are indicated. Data represents 3 biologically-independent experiments.

FIG. 3E are images of Western blots using siRNA showing that VCAM-1 expression is dependent upon FosB. HMEC-1 treated with 0.6 μM siRNA or control siRNA were stimulated with 20 ng/ml IL-1β for 2 or 4 h. Western blotting was performed with the antibodies indicated. Data is representative of 2 biologically-independent experiments. Approximate positions of molecular weight markers are shown.

FIGS. 4A-E show that BT2 inhibits ERK phosphorylation, FosB/ΔFosB and VCAM-1 expression in retinas and Matrigel plugs. Immunohistochemical staining in retinal lesions was performed for (A) pERK, (B) FosB and (C) VCAM-1. IOD of positive staining (red chromogen) was assessed using Image-Pro Plus software. Slides were photographed under 20× or 40× objective and magnified views are shown. n=3-5 per group for pERK and FosB, and n=4-6 per group for VCAM-1. Data represents the mean±SEM of the mean per animal. Statistical significance was assessed by one-way ANOVA, Kruskal-Wallis, Mann-Whitney or t-test, as appropriate. Arrows provide examples of positive staining. INL, inner nuclear layer; OPL, outer plexiform layer; ONL, outer nuclear layer; OLM, outer limiting membrane. Alternatively, Matrigel plugs were stained for (D) FosB or (E) VCAM-1. Representative FosB or VCAM-1 staining was photographed under 40× or 20× objective, respectively, with the inset providing a magnified view. Staining was quantified using Image-Pro Plus software. Data represents mean±SEM. Statistical significance was assessed by Kruskal-Wallis multiple comparisons test (FosB, n=9-11 per group) or one-way ANOVA (VCAM-1, n=10-11 per group). Arrows provide examples of positive staining. IOD denotes integrated optical density.

FIGS. 5A-D show that the carbamate moiety in BT2 is critical to its interaction with MEK1 and functional effects. FIG. 5A shows proliferation experiments in which serum-deprived HMEC-1 were treated with compound (0.4 or 0.8 μM) in medium containing 5% FBS and cell proliferation monitored using the xCELLigence system (Roche). Left, Representative growth profiles from one experiment. Right, xCELLigence data representing the mean±SEM of the means of 3 independent experiments after 79 h. Statistical significance was assessed by one-way ANOVA or Mann-Whitney test.

FIG. 5B shows HMEC-1 network formation in medium containing 1% FBS and 50 ng/ml FGF-2 combined with compound (1 μM final) and seeded in wells coated with Matrigel. Networks were quantified using NIH ImageJ software. Data represents the mean±SEM of the means of 3-4 independent experiments. Statistical significance was assessed by Kruskal-Wallis multiple comparisons test.

FIG. 5C shows SPR analysis testing the interaction of PD98059, BT2 and BT2 analogues with His-MEK1 (left panels) and His-MEK2 (right panels). Measurements were made on a Biacore T200 at 15° C. in a buffer comprising 20 mM HEPES, 150 mM NaCl, 5% DMSO pH 7.4. Data are representative of 2 independent experiments.

In FIG. 5D, HMEC-1 were treated with 1 μM compound (BT2 and analogues) in serum free medium at 37° C. for 4 h. The medium was changed to 20 ng/ml IL-1β with compound for 15 min. Lysates were resolved by SDS-PAGE and Western blotting was performed for pERK or total ERK. Data is representative of 2 biologically-independent experiments. Approximate positions of molecular weight markers are shown.

FIG. 6A shows a schematic representation of the high throughput compound screen. A luciferase-based high throughput screen was used to identify hits including use of a PAINS frequent hitter filter. Mean 1050 data and typical 11-point titration curves for BT2 and Cpd B/X/LK001 are shown.

FIG. 6B shows reactants in chemical synthesis of Cpd B/X/LK001 or BT2 analogues.

FIGS. 7A-B show that BT2, T4 and T6 inhibit endothelial FosB/ΔFosB and c-Fos expression and block cell proliferation. FIG. 7A shows band intensity (pixel intensity relative to the corresponding control) from Western blot analysis measured using NIH ImageJ software. FosB/ΔFosB band intensity was combined. Plotted data represents the values or means (where independent biological duplicates were used in the one blot)±SEM of 3 biologically-independent experiments.

FIG. 7B shows total cell numbers and % living cells as a proportion of total cells determined by Trypan Blue exclusion using a Countess II Automated Cell Counter. Countess data represents the mean±SEM of the means of 4 independent experiments. Statistical significance was assessed by Kruskal-Wallis multiple comparisons test.

FIGS. 8A-C shows immunohistochemical staining with primary antibody omitted. FIG. 8A shows immunohistochemical staining (vehicle group) using the MACH3 AP-Polymer detection system with primary antibody omitted in a region without or with lesion (arrow). Vitr, vitreous. ILM, inner limiting membrane; GCL, ganglion cell layer; IPL, inner plexiform layer; INL, inner nuclear layer; OPL, outer plexiform layer; ONL, outer nuclear layer; OLM, outer limiting membrane; IS, inner segment; OS, outer segment; RPE, retinal pigment epithelium; Chor, choroid.

FIG. 8B shows immunohistochemical staining (vehicle group) using the DAB chromogen detection system with primary antibody omitted in Matrigel plug.

FIG. 8C shows immunohistochemical staining (vehicle group) using the MACH3 AP-Polymer detection system with primary antibody omitted in Matrigel plug.

No 1º Ab denotes primary antibody omitted.

FIG. 9 shows BT2 inhibits ERK phosphorylation, FosB/ΔFosB and VCAM-1 expression. Band intensity (pixel intensity relative to the corresponding control) from Western blot analysis was measured using NIH ImageJ software. FosB/ΔFosB band intensity was combined. Plotted data represents the values or means (where independent biological duplicates were used in the one blot)±SEM of 2-3 biologically-independent experiments.

FIG. 10 shows gating of VCAM-1+ and VCAM-1⁻ cells by flow cytometry. VCAM-1+ and VCAM-1⁻ cells were gated by performing flow cytometry (FACSDiva v6.1.3) with or without primary VCAM-1 antibody (non-specific staining), respectively. Representative gating from the latter (i.e, negative control) is shown in the figure.

FIGS. 11A-C show Western blotting experiments with extracts of HMEC-1 exposed to BT2 or plasmid transfected HMEC-1. FIG. 11A shows the comparative effect of BT2 and PD98059 on IL-1β-inducible VCAM-1 expression and ERK phosphorylation. Band intensity (pixel intensity relative to the corresponding control) from Western blot analysis was measured using NIH ImageJ software. Plotted data represents the mean±SEM of 3 biologically-independent experiments.

FIG. 11B shows the comparative effect of BT2 and PD98059 (1-30 μM) on IL-β-inducible p-SAPK/JNK or p-p38. Data represents the mean±SEM of 3 biologically-independent experiments. Approximate positions of molecular weight markers are shown.

FIG. 11C shows the requirement of ERK phosphorylation in the indication of FosB and VCAM-1 expression by Western blotting. HMEC-1 rendered growth quiescent by serum deprivation (and without EGF or hydrocortisone) in 6-well plates were transfected with 6 μg of the indicated pcDNA3.1+/C-(K)DYK-based plasmid with insert ERK1 variant 1 (NM_002746.2), ERK1 variant 2 (NM_001040056.3), FosB variant 1 (NM_006732.2), FosB variant 2 (NM_001114171.2) or ΔFosB (XM_005258691.1). Western blotting was performed with total protein lysates (collected 18, 24, 48 and 72 h after plasmid transfection) using antibodies indicated. L denotes lighter exposure. Approximate positions of molecular weight markers are shown. Data is representative of 2 independent experiments.

FIG. 12 shows that BT2 is more potent than curcumin at inhibiting endothelial network formation on Matrigel. HMEC-1 in medium containing 1% FBS and 50 ng/ml FGF-2 were combined with various concentrations of BT2 or curcumin compound and seeded in wells coated with Matrigel. Networks after 4 h were quantified using NIH ImageJ software. Data represents the mean±SEM of the means of 3-4 independent experiments. Statistical significance was assessed by Kruskal-Wallis multiple comparisons test.

FIGS. 13A-B show bioactivity of structural analogues of BT2. In FIG. 13A, HMEC-1 were treated with 3 μM compound (BT2 and analogues) in serum free medium at 37° C. for 4 h. The medium was changed to 20 ng/ml IL-1β with compound for 15 min. Lysates were resolved by SDS-PAGE and Western blotting was performed for phosphorylated ERK or total ERK. Approximate positions of molecular weight markers are shown.

FIG. 13B shows HMEC-1 network formation in medium containing 1% FBS and FGF-2 combined with compound (3 μM final) and seeded in wells coated with Matrigel. Networks were quantified using NIH Image J software. Data represents the mean±SEM of the means of 3-4 independent experiments. Statistical significance was assessed by Kruskal-Wallis multiple comparisons test.

FIGS. 14A-F show that BT2 retains stability and biological activity after boiling or autoclaving. FIGS. 14A and 14B show RRLC-MS/MS analysis of heat-treated (100° C. water bath for 10 min, DL20170921-H) or non-heat treated (DL20170921) sonicated formulations of BT2 (in saline containing 0.5% Tween 80 and 0.01% DMSO) was performed in triplicate 1 or 6 weeks after preparation of the formulation. Representative chromatograms (deuterated (d3)-BT2 controls shown at right in each set) are shown.

FIGS. 14C and 14D show tubes containing BT2 or BT3 in vehicle (saline containing 0.01% DMSO and 0.5% Tween 80, sonicated) were kept at 22° C. (non heat-treated) or placed in a 100° C. water bath for 10 min then allowed to cool to 22° C. (heat-treated, +H) and freshly used or stored in the dark for 6 weeks or at least 10 months (D, black bars represent 11 months; blue bars represent 10 months; red bars represent 16 months). Serum-deprived HMEC-1 were treated with heat-treated or non heat-treated BT2 or BT3 (0.4, 0.8 μM) in medium containing 5% FBS and proliferation monitored using the xCELLigence system (Roche). Data represents the mean±SEM of the means of 3 independent experiments after 79 h. Statistical significance was assessed by one-way ANOVA.

FIGS. 14E and 14F show RRLC-MS/MS analysis of heat-treated (100° C. for 10 min) or non-heat treated sonicated formulations of BT2 (in saline containing 0.5% Tween 80 and 0.01% DMSO) was performed in triplicate 10, 11 or 16 months after preparation of the formulation. Representative chromatograms are shown.

FIG. 14G shows tubes containing BT2 in vehicle (saline containing 0.01% DMSO and 0.5% Tween 80, sonicated) that were freshly used or autoclaved (121° C., 15 psi, 20 min; +A) and stored in the dark for 4 months (orange bars). Serum-deprived HMEC-1 were treated with autoclaved or freshly used BT2 (0.4, 0.8 μM) in medium containing 5% FBS and proliferation monitored using the xCELLigence system (Roche). Proliferation data represents the mean±SEM of the means of 4 independent experiments after 79 h. Statistical significance was assessed by one-way ANOVA. Also shown is LC/MS analysis of BT2 freshly prepared or BT2 autoclaved and stored in the dark for 4 months. Figure shows total ion chromatogram integrating peak intensities of each spectrum (upper, in black) and extracted ion chromatogram integrating peak intensities of protonated precursor (m/z 327.1319-327.1361) (lower, in brown).

Table 3 provides genes induced by IL-1β (logFC ≥2) relative to control (UT) (Table 3C) and inhibited by BT2 (logFC ≥2) relative to IL-1β (Table 3A). Table 3B shows genes induced by IL-1β and inhibited by BT2. RNA-seq was performed with total RNA prepared from HMEC-1 treated with 30 μM BT2 and 4 h incubation with 20 ng/ml IL-β. These data are sourced from the same experiment represented elsewhere by heatmaps.

FIG. 15A is a graph showing the effect of various concentrations of BT2 and BT3 on monocytic cell adhesion to IL-β-treated endothelium in vitro. THP-1 adhesion to HMEC in vitro was assessed by first treating HMEC with various concentrations of BT2 or BT3 for 1 h in 96-well plates. HMEC were stimulated with 20 ng/ml IL-1β for 1 h. Fluorescence intensity of calcein labeled THP-1 that adhered to HMEC monolayers 30 min after adding the cells was then measured via fluorescent plate reader. Data is representative of 3 experiments and expressed as mean±SEM. Statistical significance was assessed by one-way ANOVA.

FIG. 15B is a graph showing the effect of various concentrations of BT2 on monocytic transendothelial cell migration toward MCP-1 in vitro. THP-1 transendothelial cell migration in vitro was assessed by treating HMEC with various concentrations of BT2 for 1 h in gelatin-coated culture inserts for 1 h. HMEC were treated with 20 ng/ml IL-1β for 1 h. THP-1 cells that had undergone transendothelial migration toward MCP-1 after 24 h was measured using a Coulter counter. Data is representative of 3 experiments and expressed as mean±SEM. Statistical significance was assessed by one-way ANOVA.

FIG. 16A provides a graph showing the effect of vehicle or BT2 at 3 mg/kg or 30 mg/kg on hindfoot thickness in a collagen antibody induced arthritic mouse model. Animals were injected i.p. with antibody cocktail on Day 0 with LPS plus BT2 (3 or 30 mg/kg in vehicle) i.p. on Day 3. Hind footpad thickness was measured using digital calipers on Day 9. Data expressed as the hind footpad thickness (mm) of each limb (left and right). n=8-10 per group. Data expressed as mean±SEM. Statistical significance was assessed by Kruskal-Wallis multiple comparisons test.

FIG. 16B provides images showing the effect of vehicle or BT2 on hindfoot thickness in a collagen antibody induced arthritic mouse model at Day 14 (gross specimens).

FIG. 16C provides images showing H&E staining of mouse footpads following no treatment, or treatment of mice with vehicle or BT2 in a collagen antibody induced arthritic mouse model at Day 14.

FIG. 16D provides a graph showing the effect of no treatment, or treatment with vehicle or BT2 on bone destruction in a collagen antibody induced arthritic mouse model. 3D Micro-CT analysis of Day 14 hind limbs was quantified where a score of 0=no bone destruction and 1=destruction was given to each individual limb. Data is expressed as mean bone destruction score per hind limb (left and right)±SEM. n=8-10 per group. Statistical significance was assessed using Firth's penalized likelihood method test.

FIG. 16E shows Micro-CT images of Day 14 hind limbs in a collagen antibody induced arthritic mouse model following no treatment, or treatment with vehicle or BT2. Arrows denote bone erosion and/or remodeling.

FIG. 16F shows graphs and images showing tartrate-resistant acid phosphatase (TRAP) staining in Day 14 hind limb osteoclasts from joints of a collagen antibody induced arthritic mouse model untreated, or treated with vehicle or BT2. Arrows provide examples of positive staining. Slides were photographed under 20× or 40× objectives. IOD of positive staining (red chromogen) was assessed using Image-Pro Plus software. Alternatively numbers of osteoclasts were counted using NIH Image J. Data represents the mean±SEM of the means. Statistical significance was assessed by Wilcoxon signed-rank test. n=6-10 per group.

FIG. 16G shows immunohistochemical staining for VCAM-1 or ICAM-1 in Day 14 hind limbs. IOD/μm² under 20× objective was assessed using Image-Pro Plus software. Data represents the mean±SEM of the means. n=3-5 per group. Statistical significance was assessed by one-way ANOVA.

### DETAILED DESCRIPTION

AP-1 is a transcription factor that regulates gene expression in response to a range of pathologic stimuli including cytokines, growth factors, stress, and viral and bacterial infection. AP-1 is a heterodimer formed through the dimerization of proteins belonging to the c-Fos, c-Jun, ATF (activating transcription factor) and/or JDP (Jun dimerization protein 2) protein families. AP-1 family member c-fos and c-jun expression and DNA binding activity has been observed in human rheumatoid synovium and is associated with disease activity, and have been shown to regulate gene products implicated in angiogenesis, while IL-1β is a mediator of bone and cartilage damage in rheumatoid arthritis. Further, AP-1 factors are expressed in retinal cells after retinal detachment and are elevated in diabetic human retina. AP-1 therefore represents an important therapeutic target for a range of diseases.

As described in the Examples, the inventor has identified and synthesised compounds of formula I and II having the ability to inhibit AP-1 dependent gene expression. The inventor has further found that these compounds inhibit phosphorylation of ERK1/2, and therefore inhibit ERK1/2-dependent gene expression.

As further described in the Examples, the inventor has shown that compounds of formula I and II inhibit: serum-inducible endothelial cell proliferation and migration; endothelial wound repair after in vitro injury; and microtubule formation on reconstituted basement membrane matrix. The inventor has further found that these compounds inhibit FosB/ΔFosB and c-Fos expression.

Accordingly, one aspect provides a method of reducing vascular permeability, angiogenesis, inflammation, cell migration and/or cell proliferation in a subject, comprising administering an effective amount of an inhibitor of FosB/ΔFosB expression. In one embodiment, the inhibitor is a compound that inhibits FosB/ΔFosB expression.

Another aspect provides a method of treating or preventing a condition associated with vascular permeability, angiogenesis, inflammation, cell migration and/or cell proliferation, comprising administering an effective amount of an inhibitor of FosB/ΔFosB expression. In one embodiment, the inhibitor is a compound that inhibits FosB/ΔFosB expression.

As described in the Examples, the inventor has further found that compound BT2 (a compound of formula II), in addition to inhibiting FosB/ΔFosB expression, inhibits phosphorylation of ERK1 and ERK2 (ERK1/2), and inhibits VCAM-1 expression, and VEGF-A expression.

Accordingly, another aspect provides a method of reducing vascular permeability, angiogenesis, inflammation, cell migration and/or cell proliferation in a subject, comprising administering an effective amount of an inhibitor of ERK1/2 phosphorylation, and/or FosB/ΔFosB expression and/or VCAM-1 expression. In one embodiment, the inhibitor is a compound that inhibits ERK1/2 phosphorylation, and FosB/ΔFosB expression and VCAM-1 expression. FosB is a leucine zipper protein family member of the Fos protein family that can dimerise with proteins of the c-Jun protein family to form AP-1. ΔFosB is a truncated splice variant of FosB. ERK1 and ERK2 are mitogen activated protein kinases (MAP kinases) that are involved in cellular functions in response to activation of surface receptors, such as surface tyrosine kinases. ERK1 and ERK2 are related serine/threonine kinases that participate in the Ras-Ras-MEK-ERK signal transduction cascade. MEK1/2 catalyses the phosphorylation of ERK1/2 at amino acid residues Tyr204 and 187 and Thr202 and 185. Following activation, ERK1/2 catalyses the phosphorylation of hundreds of cytoplasmic and nuclear proteins. The Ras-Ras-MEK-ERK signal transduction cascade is believed to play a central role in regulating a number of cellular processes including cell proliferation, adhesion, migration, differentiation, and angiogenesis.

VCAM-1 (also known as CD106) is a cell adhesion molecule expressed on blood vessels following stimulation with cytokines. In particular, VCAM-1 is upregulated in endothelial cells in response to stimulation with, for example, TNF-alpha or IL-113.

As used herein, an inhibitor of FosB/ΔFosB expression is a compound or agent which reduces the amount of FosB/ΔFosB protein produced by a cell or tissue following contact with the compound or agent relative to the amount of FosB/ΔFosB protein produced by a cell or tissue which has not been contacted with the compound or agent. An inhibitor of ERK1/2 phosphorylation is a compound or agent which reduces the extent of ERK1/2 phosphorylation in a cell or tissue following contact with the compound or agent relative to the extent of ERK1/2 phosphorylation in a cell or tissue that has not been contacted with the compound or agent. An inhibitor of VCAM-1 expression is a compound or agent which reduces the amount of VCAM-1 protein produced by a cell or tissue following contact with the compound or agent relative to the amount of VCAM-1 protein produced by a cell or tissue which has not been contacted with the compound or agent. An inhibitor of VEGF-A expression is a compound or agent which reduces the amount of VEGF-A, typically VEGF-A₁₆₅, protein produced by a cell or tissue following contact with the compound or agent relative to the amount of VEGF-A protein produced by a cell or tissue which has not been contacted with the compound or agent.

In one embodiment, the compound is an inhibitor of FosB/ΔFosB expression.

In one embodiment, the compound is an inhibitor of VCAM-1 expression.

In one embodiment, the compound is an inhibitor of ERK1/2 phosphorylation.

In one embodiment, the compound is an inhibitor of FosB/ΔFosB expression and ERK1/2 phosphorylation.

In one embodiment, the compound is an inhibitor of FosB/ΔFosB and VCAM-1 expression.

In one embodiment, the compound is an inhibitor of ERK1/2 phosphorylation,

FosB/ΔFosB expression and VCAM-1 expression.

In one embodiment, the compound is an inhibitor of ERK1/2 phosphorylation, FosB/ΔFosB expression, VCAM-1 expression and VEGF-A expression.

In one embodiment, the compound is an inhibitor of ERK1/2 phosphorylation, FosB/ΔFosB expression, VCAM-1 expression, and VEGF-A expression.

In one embodiment, the compound does not inhibit SAPK/JNK or p38 phosphorylation.

Typically, the compound is a small molecule inhibitor.

In one embodiment, the compound comprises a carbamate moiety.

In one embodiment, the compound is a dibenzoxazepinone or a benzophenone.

In one embodiment, the compound is a compound of formula I or II, or a pharmaceutically acceptable salt thereof. A compound of formula I is:

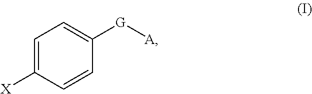

wherein:

X is F, Cl, Br or I;

G is C═O or C═N—OH; and

A is:

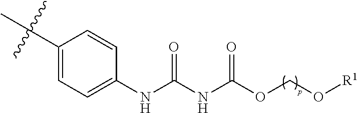

wherein p is 1, 2, 3 or 4; and R¹ is straight or branched C₁-C₆ alkyl;

or A is:

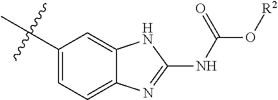

wherein R² is straight or branched C₁-C₆ alkyl.

A compound of formula II is:

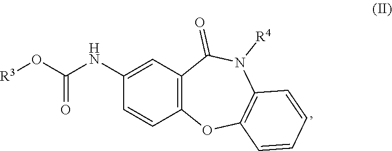

wherein:

R³ is straight or branched C₁-C₆ alkyl; and

R⁴ is straight or branched C₁-C₆ alkyl,

or R⁴ is

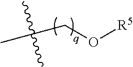

wherein q is 1, 2, 3 or 4; and R⁵ is straight or branched C₁-C₆ alkyl.

In some embodiments, the compound that reduces AP-1-dependent gene expression and/or MEK1-dependent gene expression and/or ERK1/2-dependent gene expression and/or ERK1/2 phosphorylation, and/or FosB/ΔFosB expression and/or VCAM-1 expression and/or VEGF-A expression is a compound of formula I, or a pharmaceutically acceptable salt thereof:

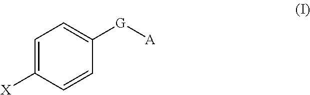

wherein:

X is F, Cl, Br or I;

G is C═O or C═N—OH; and

A is:

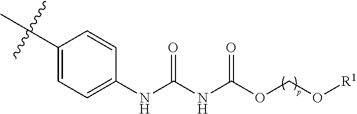

wherein p is 1, 2, 3 or 4; and R¹ is straight or branched C₁-C₆ alkyl;

or A is:

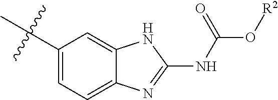

wherein R² is straight or branched C₁-C₆ alkyl.

In some embodiments of formula (I), X is F. In some embodiments of formula (I), X is Cl. In some embodiments of formula (I), X is Br. In some embodiments of formula (I), X is I. Typically, X is F or Cl.

In some embodiments of formula (I), G is C═O. In some embodiments of formula (I), G is C═N—OH.

In some embodiments of formula (I), A is

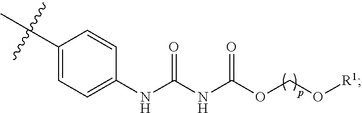

wherein p is 1, 2, 3 or 4; and R¹ is straight or branched C₁-C₆ alkyl. In some embodiments, p is 2. In some embodiments, R¹ is —CH₃. In some embodiments, p is 2 and R¹ is —CH₃.

In some embodiments of formula (I), A is

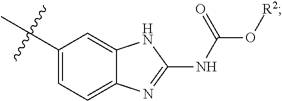

wherein R² is straight or branched C₁-C₆ alkyl. In some embodiments, R² is —CH₃.

In some embodiments, the compound of formula (I) may be a compound of formula (1-1):

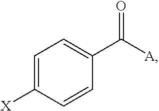

or a pharmaceutically acceptable salt thereof,
(I-1)

wherein:

X is F, Cl, Br or I; and

A is:

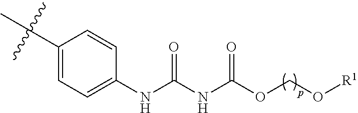

wherein p is 1, 2, 3 or 4; and R¹ is straight or branched C₁-C₆ alkyl;

or A is:

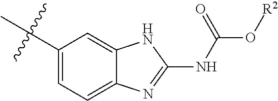

wherein R² is straight or branched C₁-C₆ alkyl.

In some embodiments, the compound of formula (1-1) may be a compound of formula (1-1a):

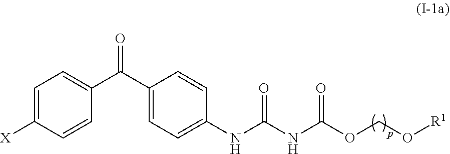

wherein:

X is F, Cl, Br or I;

p is 1, 2, 3 or 4; and

R¹ is straight or branched C₁-C₆ alkyl.

For example, the compound of formula (I-1a) may be:

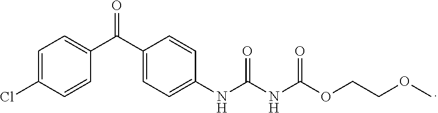 [SMILES: COCCOC(=O)NC(=O)Nc1ccc(C(=O)c2ccc(Cl)cc2)cc1]

In some embodiments, the compound of formula (1-1) may be a compound of formula (1-1b):

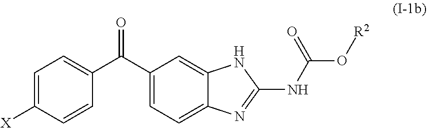

wherein:

X is F, Cl, Br or I; and

R² is straight or branched C₁-C₆ alkyl.

In one embodiment, the compound of formula (1-1b) is

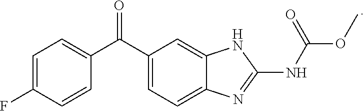 [SMILES: COC(=O)Nc1nc2ccc(C(=O)c3ccc(F)cc3)cc2[nH]1]

(also referred to herein as T6)

In some embodiments, the compound of formula (I) may be a compound of formula (1-2):

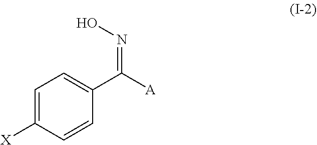

wherein:

X is F, Cl, Br or I; and

A is:

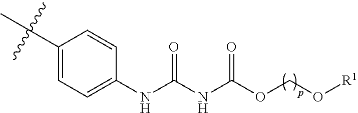

wherein p is 1, 2, 3 or 4; and R¹ is straight or branched C₁-C₆ alkyl;

or A is:

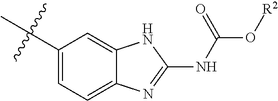

wherein R² is straight or branched C₁-C₆ alkyl.

In some embodiments, the compound of formula (1-2) may be a compound of formula (1-2a):

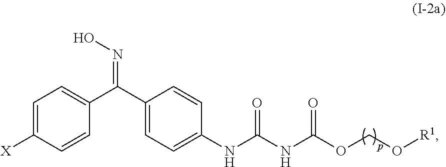

wherein:

X is F, Cl, Br or I;

p is 1, 2, 3 or 4; and

R¹ is straight or branched C₁-C₆ alkyl.

In one embodiment, the compound of formula (1-2a) is:

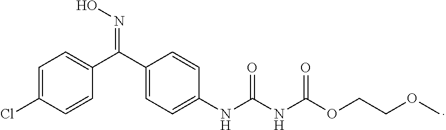 [SMILES: COCCOC(=O)NC(=O)Nc1ccc(/C(=N/O)c2ccc(Cl)cc2)cc1]

(also referred to herein as T4)

In some embodiments, the compound that reduces AP-1-dependent gene expression and/or ERK1/2-dependent gene expression and/or ERK1/2 phosphorylation, and/or FosB/ΔFosB expression and/or VCAM-1 expression and/or VEGF-A expression is a compound of formula (II), or a pharmaceutically acceptable salt thereof:

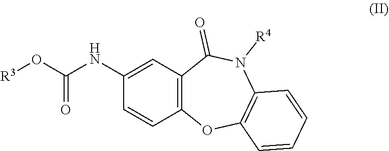

wherein:

R³ is straight or branched 01-C₆ alkyl; and

R⁴ is straight or branched 01-C₆ alkyl,

or R⁴ is

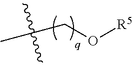

wherein q is 1, 2, 3 or 4; and R⁵ is straight or branched C₁-C₆ alkyl.

In some embodiments of formula (II), R³ is straight C₁-C₆ alkyl or branched C₁-C₆ alkyl. In some embodiments of formula (II), R³ is —CH₂CH₃ or —CH₂CH(CH₃)₂.

In some embodiments of formula (II), R⁴ is straight C₁-C₆ alkyl or branched C₁-C₆ alkyl. In some embodiments of formula (II), R⁴ is —CH₂CH 3 or —CH₂CH(CH₃)₂.

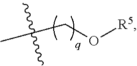

In some embodiments of formula (II), R⁴ is wherein q is 1, 2, 3 or 4; and R⁵ is straight C₁-C₆ alkyl or branched C₁-C₆ alkyl. In some embodiments of formula (II), q is 2. In some embodiments of formula (II), R⁵ is —CH₃. In some embodiments of formula (II), q is 2 and R⁵ is —CH₃.

In some embodiments, the compound of formula (II) may be a compound of formula (II-1):

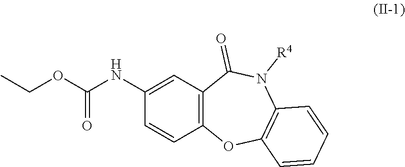

wherein:

R⁴ is straight or branched 01-C₆ alkyl;

or R⁴ is:

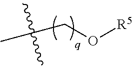

wherein q is 1, 2, 3 or 4; and R⁵ is straight or branched C₁-C₆ alkyl.

For example, the compound of formula (II-1) may be selected from:

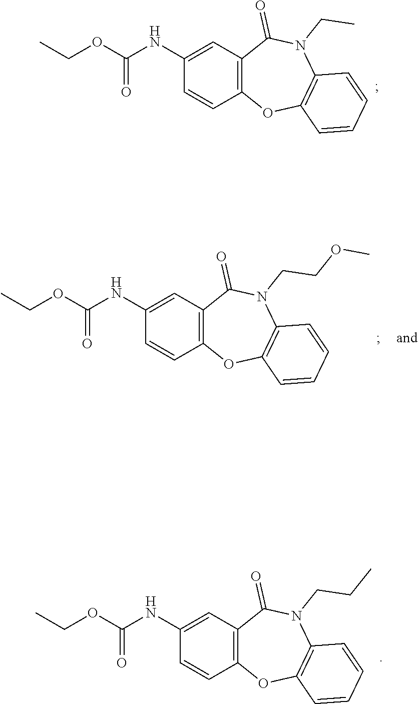

In some embodiments, the compound of formula (II) may be a compound of formula (II-2):

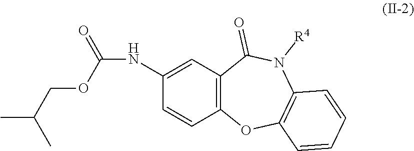

wherein:

R⁴ is straight or branched C₁-C₆ alkyl;

or R⁴ is:

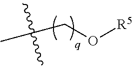

wherein q is 1, 2, 3 or 4; and R⁵ is straight or branched C₁-C₆ alkyl.

For example, the compound of formula (II-2) may be:

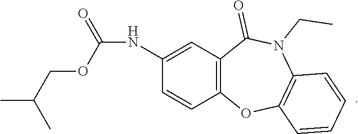 [SMILES: CCN1C(=O)c2cc(NC(=O)OCC(C)C)ccc2Oc2ccccc21]

In one embodiment, the compound of formula (II) is:

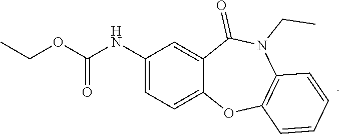 [SMILES: CCOC(=O)Nc1ccc2c(c1)C(=O)N(CC)c1ccccc1O2]

(also referred to herein as BT2)

In some embodiments, the compound which reduces AP-1-dependent gene expression and/or ERK1/2-dependent gene expression and/or ERK1/2 phosphorylation, and/or FosB/ΔFosB expression and/or VCAM-1 expression and/or VEGF-A expression is selected from:

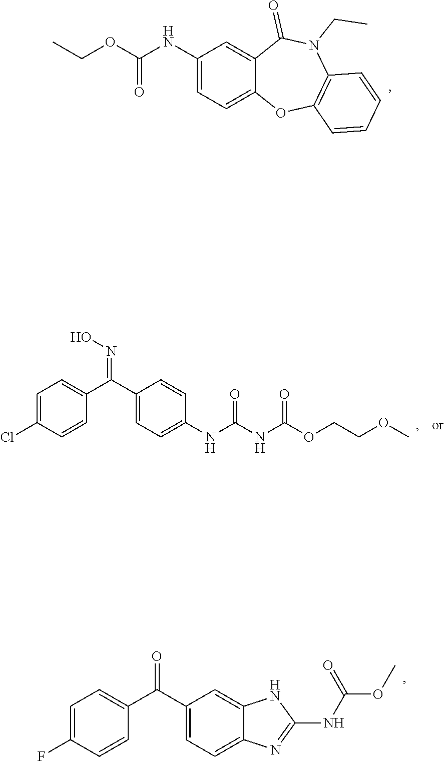 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

In another aspect, there is provided a method of reducing vascular permeability, neovascularisation, angiogenesis, inflammation, cell migration and/or proliferation in a subject, comprising administering an effective amount of a compound of formula I or II, or a pharmaceutically acceptable salt thereof.

Another aspect provides a method of treating or preventing a condition associated with vascular permeability, neovascularisation, angiogenesis, inflammation, cell migration and/or cell proliferation, comprising administering an effective amount of a compound of formula I or II, or a pharmaceutically acceptable salt thereof.

In one aspect, there is provided a method of treating or preventing a condition associated with vascular permeability, neovascularisation, angiogenesis, inflammation, cell migration and/or cell proliferation, comprising administering an effective amount of a compound selected from:

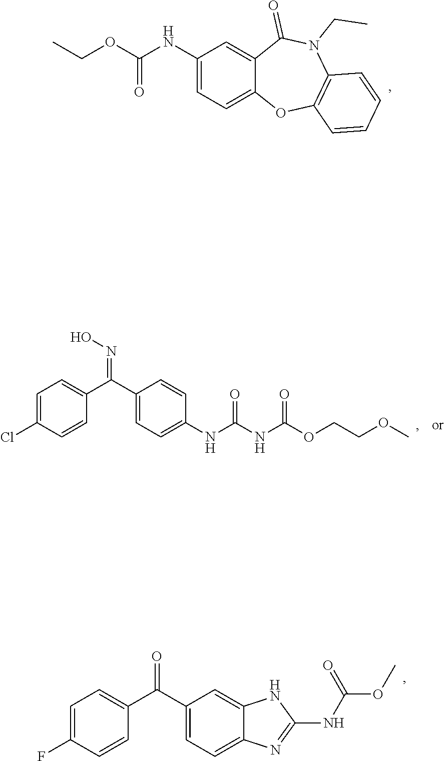 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

In one embodiment, the compound is a compound of formula:

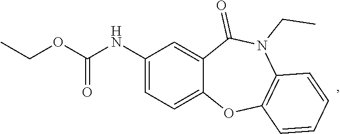 [SMILES: CCOC(=O)Nc1ccc2c(c1)C(=O)N(CC)c1ccccc1O2]

or a pharmaceutically acceptable salt thereof.

In one embodiment, the compound is a compound of formula:

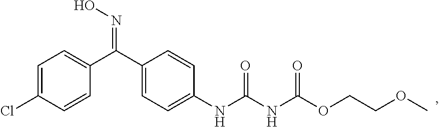 [SMILES: COCCOC(=O)NC(=O)Nc1ccc(/C(=N/O)c2ccc(Cl)cc2)cc1]

or a pharmaceutically acceptable salt thereof.

In one embodiment, the compound is a compound of formula:

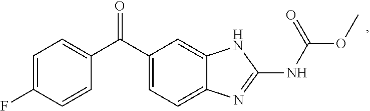 [SMILES: COC(=O)Nc1nc2ccc(C(=O)c3ccc(F)cc3)cc2[nH]1]

or a pharmaceutically acceptable salt thereof.

Another aspect provides a compound of the following formula:

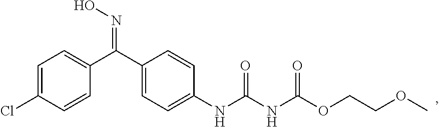 [SMILES: COCCOC(=O)NC(=O)Nc1ccc(/C(=N/O)c2ccc(Cl)cc2)cc1]

or a pharmaceutically acceptable salt thereof.

In one aspect, there is provided a method of reducing AP-1-dependent gene expression and/or ERK1/2-dependent gene expression and/or ERK1/2 phosphorylation and/or FosB/ΔFosB expression and/or VCAM-1 expression and/or VEGF-A expression in a cell, comprising administering an effective amount of a compound of formula I or II, or a pharmaceutically acceptable salt thereof. In some embodiments, the cell is the cell of a subject.

Another aspect provides a method of reducing AP-1-dependent gene expression and/or ERK1/2-dependent gene expression and/or ERK1/2 phosphorylation, and/or FosB/ΔFosB expression and/or VCAM-1 expression and/or VEGF-A expression in a cell, comprising contacting the cell with an effective amount of a compound of formula I or II, or a pharmaceutically acceptable salt thereof. In some embodiments, the cell is the cell of a subject.

Another aspect provides a method of reducing AP-1-dependent gene expression and/or ERK1/2-dependent gene expression and/or ERK1/2 phosphorylation and/or FosB/ΔFosB expression and/or VCAM-1 expression and/or VEGF-A expression in a cell, comprising contacting the cell with an effective amount of a compound selected from:

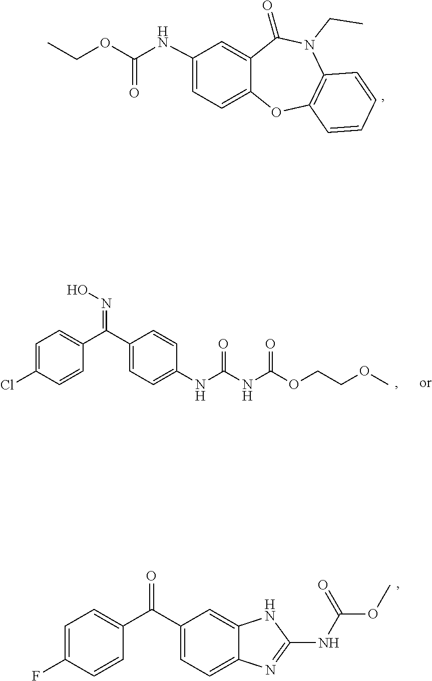 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.
In one embodiment, the compound which reduces AP-1-dependent gene expression and/or ERK1/2-dependent gene expression and/or ERK1/2 phosphorylation, and/or FosB/ΔFosB expression and/or VCAM-1 expression and/or VEGF-A expression is

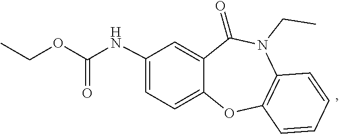 [SMILES: CCOC(=O)Nc1ccc2c(c1)C(=O)N(CC)c1ccccc1O2]

or a pharmaceutically acceptable salt thereof.

In one embodiment, AP-1-dependent gene expression and/or ERK1/2-dependent gene expression and/or ERK1/2 phosphorylation and/or FosB/ΔFosB expression and/or VCAM-1 expression and/or VEGF-A expression is reduced in the cell of a subject. In another embodiment, AP-1-dependent gene expression and/or ERK1/2-dependent gene expression and/or ERK1/2 phosphorylation, and/or FosB/ΔFosB expression and/or VCAM-1 expression and/or VEGF-A expression is reduced in a cell in vitro.

Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, trihaloacetic (e.g. trifluoroacetic), methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In one embodiment, the compound of Formula I or II, or a pharmaceutically acceptable salt thereof, is deuterated.

In one embodiment, the compound of Formula I or II, or a pharmaceutically acceptable salt thereof, is an E isomer.

In one embodiment, the compound of formula I or II, or a pharmaceutically acceptable salt thereof, is a Z isomer.

In one embodiment, the compound of formula I or II, or a pharmaceutically acceptable salt thereof, is a mixture of an E isomer and a Z isomer.

Described herein is a pharmaceutical composition comprising a compound of formula I or II, or a pharmaceutically acceptable salt thereof.

In one embodiment, there is provided a pharmaceutical composition comprising a compound of the following formula:

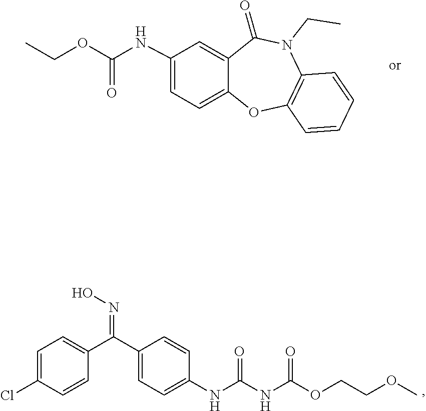 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1]

or a pharmaceutically acceptable salt thereof.

In one embodiment, the pharmaceutical composition comprises the compound:

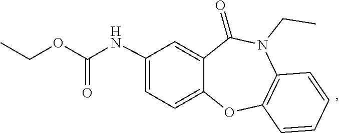 [SMILES: CCOC(=O)Nc1ccc2c(c1)C(=O)N(CC)c1ccccc1O2]

or a pharmaceutically acceptable salt thereof.

In another embodiment, the pharmaceutical composition comprises the compound:

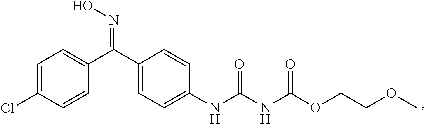 [SMILES: COCCOC(=O)NC(=O)Nc1ccc(/C(=N/O)c2ccc(Cl)cc2)cc1]

or a pharmaceutically acceptable salt thereof.

The pharmaceutical composition of the present invention may be used in the methods of the invention described herein.

The pharmaceutically composition typically comprises a pharmaceutically acceptable carrier.

The compounds of formula I and II may be used to treat any diseases or conditions mediated by AP-1 and/or ERK1/2 and/or FosB/ΔFosB, and/or VCAM-1, and/or VEGF-A, and/or IL-1p. A disease or condition is mediated by a protein or protein complex if activity of that protein or protein complex is required for development of, and/or maintaining, the disease or condition.

The compounds of formula I and II may be used to treat or prevent diseases or conditions associated with vascular permeability, neovascularisation, angiogenesis, inflammation, cell migration and/or cell proliferation.

In one embodiment, the disease or condition is associated with vascular permeability. Vascular permeability is a key feature in many disease processes including acute and chronic inflammation, wound healing and cancer during pathological angiogenesis. Vascular permeability causes retinal leakage which leads to macular edema in diabetic retinopathy, and inflammation in rheumatoid arthritis.

In some embodiments, the disease or condition associated with vascular permeability, neovascularisation, angiogenesis, inflammation, cell migration and/or cell proliferation is a disease or condition mediated by AP-1, and/or FosB/ΔFosB and/or ERK1/2 and/or VCAM-1 and/or VEGF-A and/or IL-1β.

A disease or condition associated with vascular permeability, neovascularization, angiogenesis, inflammation, cell migration and/or cell proliferation includes, for example, retinal vascular permeability, diabetic retinopathy, macula edema, rheumatoid arthritis, tissue edema, inflammation (acute and chronic), stenosis, tissue damage in myocardial infarction, age-related macular degeneration, pulmonary fibrosis, pulmonary inflammation, atherosclerosis, myocardial infarction, peripheral vascular disease, stroke.

Accordingly, in some embodiments, the disease or condition associated with vascular permeability, neovascularization, angiogenesis, inflammation, cell migration and/or cell proliferation is selected from the group consisting of:
arthritis;
rheumatoid arthritis;
bone destruction;
age-related macular degeneration;
diabetic retinopathy;
macular edema;
vascular leakage;
retinal vascular permeability;
endothelial cell dysfunction;
atherosclerosis;
stroke;
myocardial infarction;
peripheral vascular disease;
stenosis;
restenosis;
cytokine storm;
pulmonary inflammation;
pulmonary fibrosis.

As described in the Examples, the inventor has shown that administration of compound BT2 inhibits or reduces vascular permeability induced by VEGFA165, and inhibits or reduces laser induced vascular leakiness in the eye. Further, the inventor has shown that administration of BT2 reduces inflammation and bone destruction in a collagen antibody-induced arthritis model.

In one aspect, there is provided a method of treating or preventing a disease or condition of the eye associated with vascular permeability, comprising administering an effective amount of the compound of formula II, or a pharmaceutically acceptable salt thereof.

In one aspect, there is provided a method of treating or preventing retinal vascular permeability in a subject in need thereof, comprising administering an effective amount of the compound of formula II, or a pharmaceutically acceptable salt thereof.

In one aspect, there is provided a method of treating or preventing diabetic retinopathy in a subject in need thereof, comprising administering an effective amount of the compound of formula II, or a pharmaceutically acceptable salt thereof.

In one aspect, there is provided a method of treating or preventing macula edema in a subject in need thereof, comprising administering an effective amount of the compound of formula II, or a pharmaceutically acceptable salt thereof.

In one aspect, there is provided a method of treating or preventing age-related macular degeneration in a subject in need thereof, comprising administering an effective amount of the compound of formula II, or a pharmaceutically acceptable salt thereof.

In one aspect, there is provided a method of treating or preventing bone destruction and/or arthritis in a subject in need thereof, comprising administering an effective amount of the compound of formula II, or a pharmaceutically acceptable salt thereof.

In one aspect, there is provided a method of treating or preventing Rheumatoid arthritis in a subject in need thereof, comprising administering an effective amount of the compound of formula II, or a pharmaceutically acceptable salt thereof.

In one aspect, there is provided a method of treating or reducing chronic or acute inflammation in a subject in need thereof, comprising administering an effective amount of the compound of formula II, or a pharmaceutically acceptable salt thereof.

In one aspect, there is provided a method of reducing angiogenesis in a subject in need thereof, comprising administering an effective amount of the compound of formula II, or a pharmaceutically acceptable salt thereof.

In one aspect, there is provided a method of treating or reducing endothelial cell dysfunction in a subject in need thereof, comprising administering an effective amount of the compound of formula II, or a pharmaceutically acceptable salt thereof.

In one aspect, there is provided a method of treating or reducing tissue edema in a subject in need thereof, comprising administering an effective amount of the compound of formula II, or a pharmaceutically acceptable salt thereof.

In one aspect, there is provided a method of treating or reducing stenosis in a subject in need thereof, comprising administering an effective amount of the compound of formula II, or a pharmaceutically acceptable salt thereof.

In one aspect, there is provided a method of treating or reducing pulmonary fibrosis in a subject in need thereof, comprising administering an effective amount of the compound of formula II, or a pharmaceutically acceptable salt thereof.

In one aspect, there is provided a method of treating or reducing pulmonary inflammation in a subject in need thereof, comprising administering an effective amount of the compound of formula II, or a pharmaceutically acceptable salt thereof.

In one aspect, there is provided a method of treating or reducing atherosclerosis in a subject in need thereof, comprising administering an effective amount of the compound of formula II, or a pharmaceutically acceptable salt thereof.

In one aspect, there is provided a method of treating or reducing myocardial infarction in a subject in need thereof, comprising administering an effective amount of the compound of formula II, or a pharmaceutically acceptable salt thereof.

In one aspect, there is provided a method of treating or reducing peripheral vascular disease in a subject in need thereof, comprising administering an effective amount of the compound of formula II, or a pharmaceutically acceptable salt thereof.

In one aspect, there is provided a method of treating or reducing stroke in a subject in need thereof, comprising administering an effective amount of the compound of formula II, or a pharmaceutically acceptable salt thereof.

In some embodiments, the compound of formula (II) may be a compound of formula (II-1):

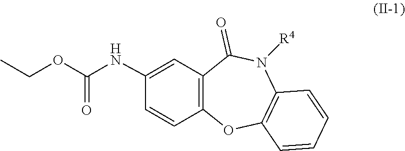

wherein:

R⁴ is straight or branched C₁-C₆ alkyl;

or R⁴ is:

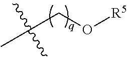

wherein q is 1, 2, 3 or 4; and R⁵ is straight or branched C₁-C₆ alkyl.

For example, the compound of formula (II-1) may be selected from:

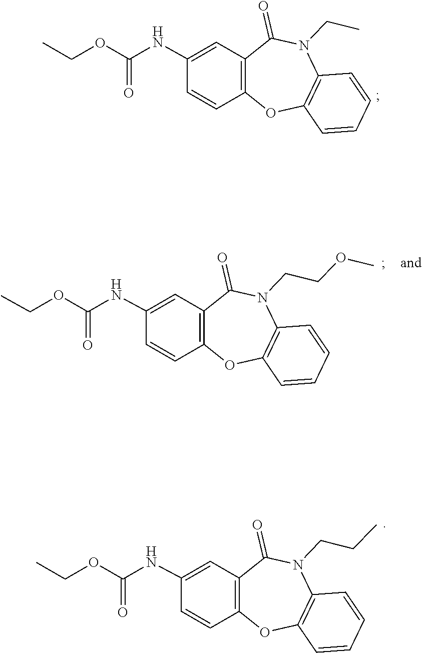

In some embodiments, the compound of formula (II) may be a compound of formula (II-2):

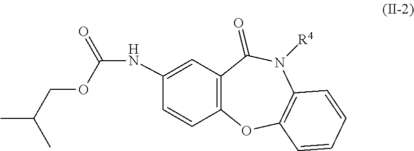

wherein:

R⁴ is straight or branched C₁-C₆ alkyl;

or R⁴ is:

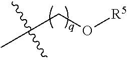

wherein q is 1, 2, 3 or 4; and R⁵ is straight or branched C₁-C₆ alkyl.

For example, the compound of formula (II-2) may be:

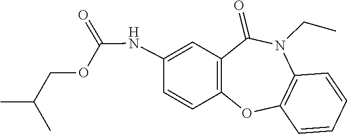 [SMILES: CCN1C(=O)c2cc(NC(=O)OCC(C)C)ccc2Oc2ccccc21]

Typically, the compound of formula (II) is:

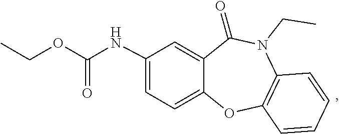 [SMILES: CCOC(=O)Nc1ccc2c(c1)C(=O)N(CC)c1ccccc1O2]

or a pharmaceutically acceptable salt thereof.
(BT2)

In one aspect, there is provided a method of treating or preventing a disease or condition of the eye associated with vascular permeability, comprising administering an effective amount of BT2, or a pharmaceutically acceptable salt thereof.

In one embodiment, there is provided a method of treating or preventing retinal vascular permeability in a subject in need thereof, comprising administering an effective amount of BT2, or a pharmaceutically acceptable salt thereof.

In one embodiment, there is provided a method of treating or preventing diabetic retinopathy in a subject in need thereof, comprising administering an effective amount of

BT2, or a pharmaceutically acceptable salt thereof.

In one embodiment, there is provided a method of treating or preventing macula edema in a subject in need thereof, comprising administering an effective amount of BT2, or a pharmaceutically acceptable salt thereof.

In one embodiment, there is provided a method of treating or preventing age-related macular degeneration in a subject in need thereof, comprising administering an effective amount of BT2, or a pharmaceutically acceptable salt thereof.

In one embodiment, there is provided a method of treating or preventing bone destruction and/or arthritis in a subject in need thereof, comprising administering an effective amount of BT2, or a pharmaceutically acceptable salt thereof.

In one embodiment, there is provided a method of treating or preventing rheumatoid arthritis in a subject in need thereof, comprising administering an effective amount of BT2, or a pharmaceutically acceptable salt thereof.

In one embodiment, there is provided a method of treating or reducing chronic or acute inflammation in a subject in need thereof, comprising administering an effective amount of BT2, or a pharmaceutically acceptable salt thereof.

In one embodiment, there is provided a method of reducing angiogenesis in a subject in need thereof, comprising administering an effective amount of BT2, or a pharmaceutically acceptable salt thereof.

In one embodiment, there is provided a method of treating or reducing endothelial cell dysfunction in a subject in need thereof, comprising administering an effective amount of BT2, or a pharmaceutically acceptable salt thereof.

In one embodiment, there is provided a method of treating or reducing tissue edema in a subject in need thereof, comprising administering an effective amount of BT2, or a pharmaceutically acceptable salt thereof.

In one embodiment, there is provided a method of treating or reducing stenosis in a subject in need thereof, comprising administering an effective amount of BT2, or a pharmaceutically acceptable salt thereof.

In one embodiment, there is provided a method of treating or reducing pulmonary fibrosis in a subject in need thereof, comprising administering an effective amount of

BT2, or a pharmaceutically acceptable salt thereof.

In one embodiment, there is provided a method of treating or reducing pulmonary inflammation in a subject in need thereof, comprising administering an effective amount of BT2, or a pharmaceutically acceptable salt thereof.

In one embodiment, there is provided a method of treating or reducing atherosclerosis in a subject in need thereof, comprising administering an effective amount of BT2, or a pharmaceutically acceptable salt thereof.

In one embodiment, there is provided a method of treating or reducing myocardial infarction in a subject in need thereof, comprising administering an effective amount of BT2, or a pharmaceutically acceptable salt thereof.

In one embodiment, there is provided a method of treating or reducing peripheral vascular disease in a subject in need thereof, comprising administering an effective amount of the compound of formula II, or a pharmaceutically acceptable salt thereof.

In one embodiment, there is provided a method of treating or reducing stroke in a subject in need thereof, comprising administering an effective amount of the compound of formula II, or a pharmaceutically acceptable salt thereof.

The methods described herein may involve the administration of a pharmaceutical composition comprising a compound described herein or a pharmaceutically acceptable salt thereof, and a pharmaceutically acceptable carrier.

Described herein is a pharmaceutical composition comprising a compound of formula I or II, or a pharmaceutically acceptable salt thereof, and a pharmaceutically acceptable carrier.

In one embodiment, the compound of formula I or II is selected from BT2, T4 and T6.

In some embodiments, the carrier is a non-naturally occurring carrier.

In some embodiments, the compounds described herein or a pharmaceutically acceptable salt thereof may be used in combination with one or more other agents.

It will be understood that the combined administration of a compound described herein or a pharmaceutically acceptable salt thereof with the one or more other agents may be concurrent, sequential or separate administration.

The term “composition” encompasses formulations comprising the active ingredient with conventional carriers and excipients, and also formulations with encapsulating materials as a carrier to provide a capsule in which the active ingredient (with or without other carriers) is surrounded by the encapsulation carrier. In pharmaceutical compositions, the carrier is “pharmaceutically acceptable” meaning that it is compatible with the other ingredients of the composition and is not deleterious to a subject. The pharmaceutical compositions of the present invention may contain other agents or further active agents as described above, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those known in the art of pharmaceutical formulation (See, for example, Remington: The Science and Practice of Pharmacy, 21st Ed., 2005, Lippincott Williams & Wilkins).

The pharmaceutical composition may be suitable for intravitreal, oral, rectal, nasal, topical (including dermal, buccal and sub-lingual), vaginal or parenteral (including intramuscular, sub-cutaneous and intravenous) administration or in a form suitable for administration by inhalation or insufflation.

The compounds described herein or a pharmaceutically acceptable salt thereof, together with a pharmaceutically acceptable carrier, may thus be placed into the form of pharmaceutical compositions and unit dosages thereof. The pharmaceutical composition may be a solid, such as a tablet or filled capsule, or a liquid such as solution, suspension, emulsion, elixir, or capsule filled with the same, for oral administration. The pharmaceutical composition may be a liquid such as solution, suspension, or emulsion, for intravitreal administration. The pharmaceutical composition may also be in the form of suppositories for rectal administration or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds described herein, pharmaceutically acceptable carriers can be either solid or liquid. Solid form preparations include powders, tablets, pills, capsules, cachets, lozenes (solid or chewable), suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The pharmaceutical compositions according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for injectable solutions or dispersions, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredient in the required amount in the appropriate solvent with various other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

The compounds described herein may be formulated into compositions suitable for oral administration, for example, with an assimilable edible carrier, or enclosed in hard or soft shell gelatin capsule, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like.

The amount of active compound in therapeutically useful compositions should be sufficient that a suitable dosage will be obtained.

The tablets, troches, pills, capsules, lozenges, implants and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active ingredient to specific regions of the gut.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers include any and all pharmaceutically acceptable solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration, the compounds described herein may be formulated as an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions for nasal administration may be applied directly to the nasal cavity by conventional means, for example with a dropper, pipette or spray. The formulations may be provided in single or multidose form. In the case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds of the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of the active ingredient may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g. gelatin, or blister packs from which the powder may be administered by means of an inhaler.

In formulations intended for administration to the respiratory tract, including intranasal formulations, the active ingredient will generally have a small particle size for example of the order of 5 to 10 microns or less. Such a particle size may be obtained by means known in the art, for example by micronisation.

The compounds described herein can be formulated into compositions for ocular, intraocular, intravitreal or subconjunctival injection. The compounds described herein may be formulated for administration by means of eye drops, contact lens or an implant. Implants may be injected intravitreally into the eye. The implant may allow delivering constant therapeutic levels of the compound. Such slow release implants are typically made with a pelleted compound core surrounded by nonreactive substances such as silicon, ethylene vinyl acetate (EVA), or polyvinyl alcohol (PVA); these implants are nonbiodegradable and can deliver continuous amounts of a compound for months to years. Matrix implants may also be used. They are typically used to deliver a loading dose followed by tapering doses of the compound during a 1-day to 6-month time period. They are most commonly made from the copolymers poly-lactic-acid (PLA) and/or poly-lactic-glycolic acid (PLGA), which degrade to water and carbon dioxide.

Formulations for intravitreal administration may be formulated as aqueous base containing one or more emulsifying agents, stabilising agents, dispersing agents, penetrating agents, or suspending agents.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Parental compositions may be in the form of physically discrete units suited as unitary dosages for the subjects to be treated, each unit containing a predetermined quantity of the active ingredient calculated to produce the desired therapeutic effect in association a pharmaceutical carrier.

The compounds may also be administered in the absence of carrier where the compounds are in unit dosage form.

The term “effective amount” refers to the amount of a compound effective to achieve the desired response.

An effective amount of a compound described herein, or a pharmaceutically acceptable salt thereof, can be determined by a person skilled in the art having regard to the particular compound.

It will be understood that the specific dose level and frequency of dosage for any particular subject may be varied and will depend upon a variety of factors including the activity of the specific compound employed, the metabolic stability and length of action of that compound, the age, body weight, general health, sex and diet of the subject, the mode and time of administration, rate of excretion, drug combinations, and the severity of the particular condition.

Suitable dosages of the compounds described herein or further active agents administered in combination with compounds described herein can be readily determined by a person skilled in the art having regard to the particular compound of the invention or further active agent selected.

It will further be understood that when the compounds described herein are to be administered in combination with one or more agents, or other active agents, the dosage forms and levels may be formulated for either concurrent, sequential or separate administration or a combination thereof.

The methods of the present invention are intended for use with any subject that may experience the benefits of the methods of the invention. Thus, the term “subject” includes humans as well as non-human mammals. The subject may, for example, be a domestic animal, zoo animal or livestock.

The inventor also envisages that the compounds of formula I and II can be used for inhibition of AP-1-dependent gene expression and/or ERK1/2-dependent gene expression and/or ERK1/2 phosphorylation, and/or FosB/ΔFosB expression and/or VCAM-1 expression and/or VEGF-A expression in vitro, in, for example, laboratory applications.

One aspect provides a method of reducing AP-1-dependent gene expression and/or ERK1/2-dependent gene expression in a cell in vitro, comprising contacting the cell with an effective amount of a compound of formula I or II, or a pharmaceutically acceptable salt thereof.

Another aspect provides a method of reducing AP-1-dependent gene expression and/or ERK1/2-dependent gene expression in a cell in vitro, comprising contacting the cell with an effective amount of a compound selected from:

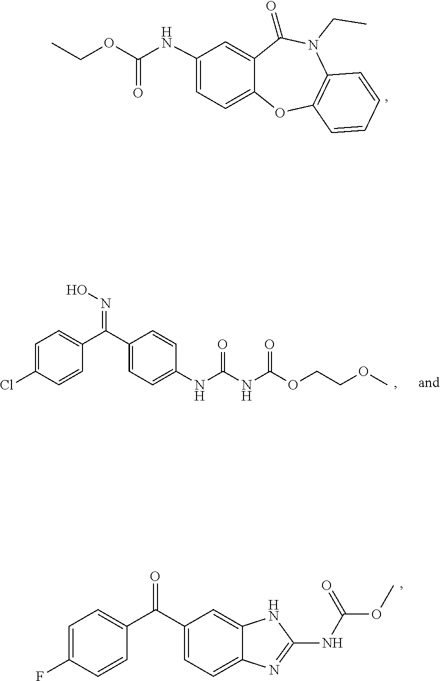 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

Another aspect provides a method of reducing ERK1/2 phosphorylation, and/or FosB/ΔFosB expression and/or VCAM-1 expression and/or VEGF-A expression in a cell in vitro, comprising contacting the cell with an effective amount of a compound of formula I or II, or a pharmaceutically acceptable salt thereof.

Another aspect provides a method of reducing ERK1/2 phosphorylation, and/or FosB/ΔFosB expression and/or VCAM-1 expression and/or VEGF-A expression in a cell in vitro, comprising contacting the cell with an effective amount of a compound selected from:

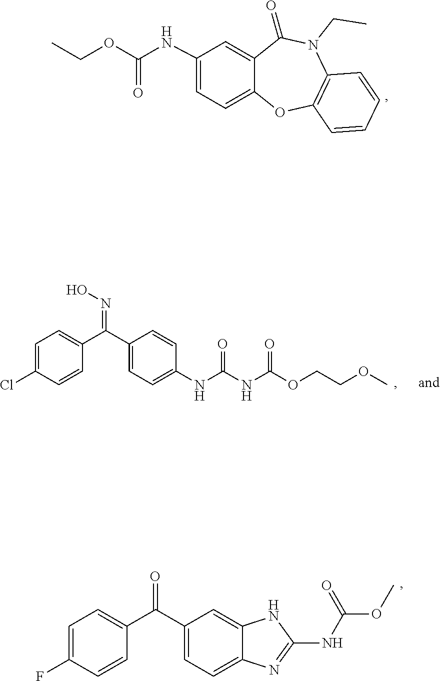 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

Another aspect provides a method of inhibiting ERK1/2 phosphorylation, comprising incubating ERK1/2 with an effective amount of a compound of formula I or II, or a pharmaceutically acceptable salt thereof.

Another aspect provides a method of inhibiting ERK1/2 phosphorylation, comprising incubating ERK1/2 with an effective amount of a compound selected from:

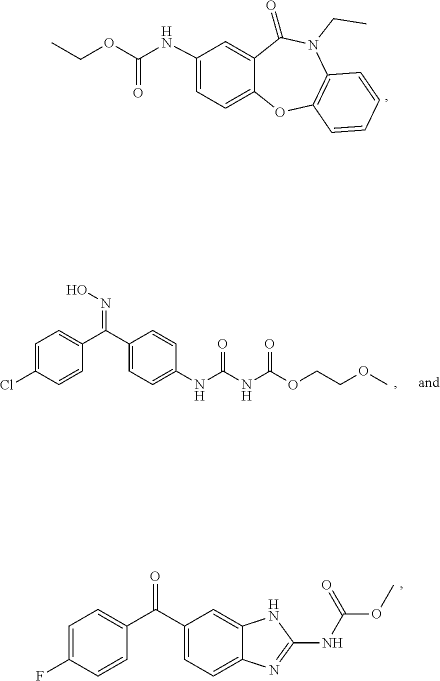 [SMILES: CCOC(=O)Nc1ccc2c(c1)C(=O)N(CC)c1ccccc1O2 ; COCCOC(=O)NC(=O)Nc1ccc(/C(=N/O)c2ccc(Cl)cc2)cc1 ; COC(=O)Nc1nc2ccc(C(=O)c3ccc(F)cc3)cc2[nH]1]

or a pharmaceutically acceptable salt thereof.

Also provide is a method of producing the compound of formula I or II, or a pharmaceutical salt thereof.

Unless otherwise herein defined, the following terms will be understood to have the general meanings which follow. The terms referred to below have the general meanings which follow when the term is used alone and when the term is used in combination with other terms, unless otherwise indicated. Hence, for example, the definition of “alkyl” applies to “alkyl” as well as the “alkyl” portions of “haloalkyl”, “heteroalkyl”, “arylalkyl” etc.

The term “alkyl” refers to a straight chain or branched chain saturated hydrocarbyl group. Unless indicated otherwise, preferred are C₁₋₆alkyl and C₁₋₄alkyl groups. The term “C_{x-y}alkyl”, where x and y are integers, refers to an alkyl group having x to y carbon atoms. For example, the term “C₁₋₆alkyl” refers to an alkyl group having 1 to 6 carbon atoms. Examples of C₁₋₆alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “alkyl” also encompasses alkyl groups containing one less hydrogen atom such that the group is attached via two positions, i.e. divalent.

As used herein, “treating” means affecting a subject, tissue or cell to obtain a desired pharmacological and/or physiological effect and includes inhibiting the condition, i.e. arresting its development; or relieving or ameliorating the effects of the condition i.e., cause reversal or regression of the effects of the condition. As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

The term “effective amount” refers to the amount of the compound that will elicit the biological or medical response of a tissue, system, animal or human that is being sought by the researcher, veterinarian, medical doctor or other clinician.

TABLE 1
Compounds referred to herein.

| Compound | | |
|---|---|---|
| name | Chemical name | Chemical structure |
| BT2 | (10-ethyl-11-oxo- 10,11-dihydro- dibenzo[b,f][1,4] oxazepin- 2-yl)-carbamic acid ethyl ester (CAS 922029-50-3) | 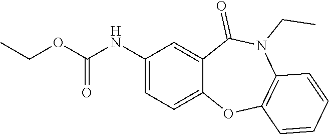 [SMILES: CCOC(=O)Nc1ccc2c(c1)C(=O)N(CC)c1ccccc1O2] |
| BT3 | 2-amino-10- ethyldibenzo[b,f][1,4] oxazepin-11 (10H)-one (CAS 23474-61-5) | 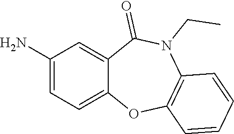 [SMILES: CCN1C(=O)c2cc(N)ccc2Oc2ccccc21] |
| CpdX/ CpdB/ LK001 | 2-methoxyethyl[[[4-(4- chlorobenzoyl)phenyl] amino]carbonyl] carbamate (CAS 351068-74-1) | 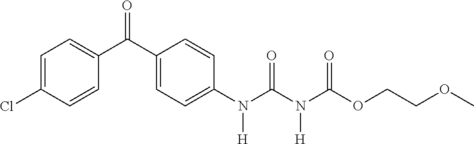 [SMILES: COCCOC(=O)NC(=O)Nc1ccc(C(=O)c2ccc(Cl)cc2)cc1] |
| T4 | 2-Methoxyethyl[[[4-(4- chlorophenyl) (hydroxyimino)methyl) phenyl]amino]carbonyl] carbamate | 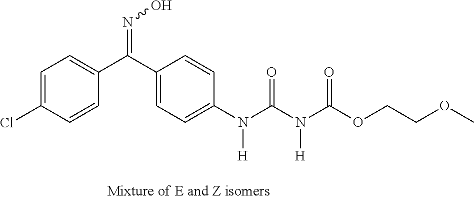 [SMILES: COCCOC(=O)NC(=O)Nc1ccc(C(=NO)c2ccc(Cl)cc2)cc1] |
| T6 | Flubendazole (CAS 31430-15-6) | 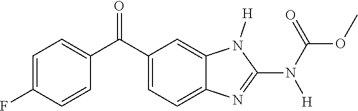 [SMILES: COC(=O)Nc1nc2ccc(C(=O)c3ccc(F)cc3)cc2[nH]1] |
| T7 | (4-aminophenyl)(4- fluorophenyl)methanone (CAS 10055-40-0) | 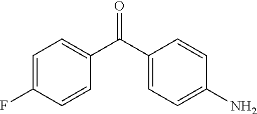 [SMILES: Nc1ccc(C(=O)c2ccc(F)cc2)cc1] |
| BT2-MeOA | N-(10-ethyl-11-oxo- 10,11- dihydrodibenzo[b,f][1,4] oxazepin-2-yl)-2- methoxyacetamide (CAS 922029-59-2) | 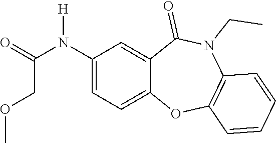 [SMILES: CCN1C(=O)c2cc(NC(=O)COC)ccc2Oc2ccccc21] |
| BT2-EOMe | ethyl (10-(2- methoxyethyl)-11-oxo- 10,11- dihydrodibenzo[b,f][1,4] oxazepin-2-yl)carbamate | 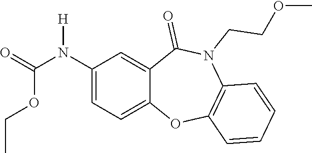 [SMILES: CCOC(=O)Nc1ccc2c(c1)C(=O)N(CCOC)c1ccccc1O2] |
| BT2-Pr | ethyl (11-oxo-10-propyl- 10,11- dihydrodibenzo[b,f][1,4] oxazepin-2-yl)carbamate (CAS 922029-50-3) | 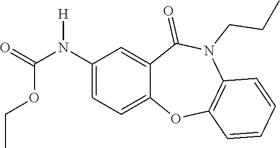 [SMILES: CCCN1C(=O)c2cc(NC(=O)OCC)ccc2Oc2ccccc21] |
| BT2-IC | isobutyl (10-ethyl-11- oxo-10,11- dihydrodibenzo[b,f][1,4] oxazepin-2-yl)carbamate | 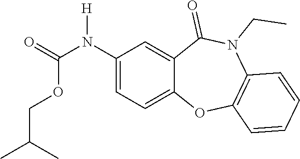 [SMILES: CCN1C(=O)c2cc(NC(=O)OCC(C)C)ccc2Oc2ccccc21] |
| BT2-IMO | ethyl (11-(oxetan-3- ylmethoxy)dibenzo[b,f] [1,4]oxazepin-2- yl)carbamate | 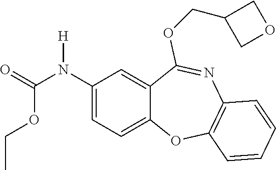 [SMILES: CCOC(=O)Nc1ccc2c(c1)C(OCC1COC1)=Nc1ccccc1O2] |
| BT2-MO | ethyl (10-(oxetan-3- ylmethyl)-11-oxo-10,11- dihydrodibenzo[b,f][1,4] oxazepin-2-yl)carbamate | 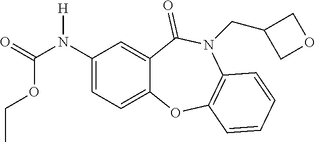 [SMILES: CCOC(=O)Nc1ccc2c(c1)C(=O)N(CC1COC1)c1ccccc1O2] |
| BT2-deut | ethyl (10-ethyl(2′,2′,2′- d3)-11-oxo-10,11- dihydrodibenzo[b,f][1.4] oxazepin-2-yl)carbamate | 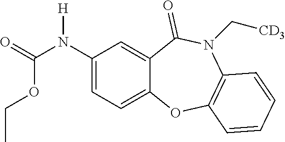 [SMILES: [2H]C([2H])([2H])CN1C(=O)c2cc(NC(=O)OCC)ccc2Oc2ccccc21] |

The compounds described herein may be synthesised by methods known in the art. The compounds referred to herein as BT2 and T6 are commercially available. For example, BT2 can be purchased from Aurora Building Blocks, USA, or Life Chemicals HTS Compounds, Canada. T6 can be purchased from, for example, Sigma-Aldrich, USA.

The present invention is further described below by reference to the following non-limiting Examples.

### Examples

Transcription factors, particularly those encoded by immediate-early genes, integrate cues from the extracellular environment with signaling and transcriptional control. While it is clear that transcription factors control disease there are no drugs on the market that directly target such factors (Mapp et al., Nature Chemical Biology 11, 891-894 (2015)). despite encouraging drug development pipelines (Miyoshi, et al., J Invest Dermatol 131, 108-117 (2011); Cho, E. A., et al., The Lancet 381, 1835-1843 (2013)). Basic region-leucine zipper (bZIP) factors comprising AP-1 regulate gene expression in response to a range of pathologic stimuli including cytokines, growth factors, stress and viral and bacterial infection (Hess, et al., Journal of Cell Science 117, 5965-5973 (2004)). AP-1 family members including FosB/ΔFosB (Chen, G., et al., Front Neurosci 11, 112 (2017)) are under the control of mitogen activated protein kinases (MAPK) (Karin, M. J Bio/Chem 270, 16483-16486 (1995)) and regulate gene expression in response to a range of pathologic stimuli including cytokines, growth factors, various stresses and viral and bacterial infection (Hess, et al., Journal of Cell Science 117, 5965-5973 (2004)). AP-1 members are elevated in diabetic human retina (Oshitari, T. at el. Current Eye Research 39, 527-531 (2014)) and expressed in retinal cells after retinal detachment (Geller, et al., Invest Ophthalmol Vis Sci 42, 1363-1369 (2001)). AP-1 DNA binding activity has also been observed in human rheumatoid synovium and is associated with disease activity (Asahara, H., et al., Arthritis Rheum 40, 912-918 (1997)) while IL-1β is a known mediator of bone and cartilage damage in RA (Duff, G. W. Cytokines and Rheumatoid Arthritis. in Clinical Applications of Cytokines: Role in Pathogenesis, Diagnosis, and Therapy (eds. Oppenheim, J. J., Rossio, J. L. & Gearing, A. J. H.) (Oxford University Press, Oxfrd, 1993). Attempts have been made to translate AP-1 inhibitors to the clinic, however patient use is hamstrung by the paucity of effective drugs.
We employed a high throughput approach to screen ˜100,000 compounds and identified a novel dibenzoxazepinone we termed BT2 which has previously never before been investigated. We found that BT2 inhibits a range of proliferative, migratory angiogenic and inflammatory processes. BT2 directly interacts preferentially with MEK1 and inhibits ERK activation, and suppresses the inducible expression of the AP-1 protein FosB/ΔFosB and that of VCAM-1 and VEGF-A₁₆₅. BT2 abrogates CD31 and tartrate-resistant acid phosphatase (TRAP) staining. BT2 also inhibits retinal vascular leakage in rats and rabbits, and suppresses inflammation and bone destruction in mice. BT2 withstands boiling and remains biologically stable for up to 16 months. Thus, BT2 is a new pharmacologic inhibitor of angiogenesis, vascular permeability and inflammation, and offers a new potential therapeutic tool for nAMD/DR and RA patients.

### Materials and Methods

High-throughput screen of compound library. Hits were selected from the ˜100,000 compound Lead Discovery Library at the HIS Facility at Walter & Eliza Hall Institute of Medical Research (WEHI, Bundoora, Vic) with a commercially-available human embryonic kidney (HEK)-293 cell-based assay in 384-well microtitre plates in which Firefly luciferase was driven by multiple copies of the AP-1 response element (293/AP-1-luc cells, Panomics, Fremont, CA). Briefly, the cell-based assay involved plating 5×10³ cells into 384-well plates in DMEM, pH 7.4 containing 10% FBS. After ˜18 h, the cells were induced with 10 ng/ml 2-O-tetradecanoylphorbol-13-acetate (TPA) (Sigma, St Louis, MO) in the absence or presence of test compound, then after ˜18 h, luciferase activity was measured using a luminometer. The hit rate of the primary screen was 2.4%. Hits were picked for single point retest in triplicate and 931 test compounds re-confirmed at greater than 50% inhibition. A substructure filter was then applied to remove pan-assay interference compounds (Baell, J. B., et al., J Med Chem 53, 2719-2740 (2010)) and using the most stringent filtering criteria 256 hits were selected for further study. After dose response testing, 24 compounds with molecular weight <400 Da were reordered from suppliers and tested in secondary assays.
Compound synthesis and purification. BT2, Cpd B/X/LK001 and structural analogues were synthesized and purified (>95%) at Advanced Molecular Technologies Pty Ltd (Scoresby, Vic) or obtained commercially as indicated below.

(10-Ethyl-11-oxo-10,11-dihydro-dibenzo[b,f][1,4]oxazepin-2-yl)-carbamic acid ethyl ester (BT2). Diethyl pyrocarbonate (22.2 ml, 24.43 g, 151 mmol) was added to 2-Amino-(BT3) (35.0 g, 137 mmol) in 100 ml of dimethylformamide (DMF), and the mixture stirred for 1 h under an atmosphere of nitrogen at 22° C. The solid was filtered and rinsed with ethyl acetate (EtOAc) (100 ml) to give a pure first crop. The combined solvent (DMF and EtOAc) was removed and the mixture was dissolved in dichloromethane (DCM) (200 ml) then washed twice with water (100 ml). The organic layer was separated, dried with MgSO₄, filtered and the solvent was removed to give a yellow solid. This solid was slurried in EtOAc and filtered to give a pure colorless solid. The crops were combined to give 35.0 g (79% yield) of a pure colorless solid. ¹H-NMR (400 MHz, D₆-DMSO): δ=1.15-1.4 (m, 6H); 4.05-4.25 (m, 4H); 7.2 (m, 3H); 7.3 (d, 1H); 7.48 (d, 1H); 7.55 (d, 1H); 7.5 (bs, 1H); 9.7 (s, 1H) ppm.

Isobutyl(10-ethyl-11-oxo-10,11-dihydrodibenzo[b,f][1,4]oxazepin-2-yl) carbamate (BT2-IC). To 2-Amino-10-methyl-10H-dibenzo[b,f][1,4]oxazepin-11-one (1.5 g, 5.89 mmol, 1.0 eq) in 50 ml of DMF under an atmosphere of nitrogen was added diisobutyl dicarbonate (1.55 g, 7.08 mmol, 1.2 eq). The mixture was stirred overnight at 40° C. (external). The solvent was removed and the mixture was dissolved in DCM (200 ml) and washed twice with water (150 ml). Then the organic layer was dried with MgSO₄, filtered on a sintered funnel and the solvent was removed to give 3.0 g of a brown solid as a crude product. This solid was purified by column chromatography on silica gel and a mixture of hexane: ethyl acetate (starting from 10% ethyl acetate in hexane, then polarity increased to 20% to give the product 1.55 g (74%) as a faint yellow solid. ¹H-NMR (400 MHz, CDCl₃) δ=0.93 (s, 3H); 0.95 (s, 3H); 1.35 (t, 3H); 1.90-2.10 (m, 1H); 3.93 (d, 2H); 4.15 (q, 2H); 6.87 (s, 1H); 7.11-7.21 (m, 3H); 7.23-7.26 (m, 1H); 7.28-7.32 (m, 1H); 7.61 (d, 1H); 7.75 (brs, 1H).

N-(10-Ethyl-11-oxo-10,11-dihydro-dibenzo[b,f][1,4]oxazepin-2-yl)-2-methoxy-acetamide (BT2-MeOA). To methoxy acetic acid (1.169 g, 0.996 ml, 12.9 mmol, 1.1 eq) in 60 ml of DMF under an atmosphere of nitrogen was added carbonyldiimidazole (2.487 g, 15.0 mmol, 1.3 eq). The mixture was stirred for 30 min. Then the 2-amino-10-methyl-10H-dibenzo[b,f][1,4] oxazepin-11-one (3.0 g, 11.8 mmol, 1.0 eq) was added and the reaction stirred at 30° C. (external) overnight. The solvent was removed, water (200 ml) and DCM (200 ml) were added to the mixture and acidified to pH 6 with 2M HCl. The organic phase was washed twice with 50 ml water. The organic layer was dried with MgSO₄, filtered on a sintered funnel and the solvent was removed to give 3.7 g of a sticky yellow solid. The crude product was purified by column chromatography using 50% ethyl acetate and in hexane to give the product 3.26 g (85%) as a faint brown solid. ¹H-NMR (400 MHz, CDCl₃): δ=1.22 (t, 3H); 3.35 (s, 3H); 3.95 (s, 2H), 4.1 (m, 2H); 7.2-7.3 (m, 3H); 7.35 (d, 1H); 7.52 (d, 1H); 7.8 (d, 1H); 8.03 (s, 1H); 9.9 (s, 1H) ppm.

(11-Oxo-10-propyl-10,11-dihydro-dibenzo[b,f][1,4]oxazepin-2-yl)-carbamic acid ethyl ester (BT2-Pr). To 2-Amino-10-propyl-10H-dibenzo[b,f][1,4]oxazepin-11-one (2.4 g, 9.43 mmol, 1.0 eq) in 70 ml of DMF under an atmosphere of nitrogen was added diethyl pyrocarbonate (2.30 g, 14.16 mmol, 1.5 eq). The mixture was stirred overnight at 40° C. (external). The solvent was removed and the mixture was dissolved in DCM (200 ml) and washed twice with water (150 ml). The organic layer was separated and dried with MgSO₄, filtered on a sintered funnel and the solvent removed to give 3.0 g of a brown solid as a crude product. This solid was purified by column chromatography in 20% ethyl acetate in hexane to give a pure compound 2.2 g (72%) as a faint yellow solid. ¹H-NMR (400 MHz, CDCl₃): δ=1.30 (t, 3H); 3.40 (s, 3H); 3.80 (t, 2H); 4.20-4.25 (m, 4H); 6.60 (s, 1H); 7.13-7.26 (m, 5H); 7.55-7.60 (m, 2H); 7.68 (s, 1H) ppm.

[10-(2-Methoxy-ethyl)-11-oxo-10,11-dihydro-dibenzo[b,f][1,4] oxazepin-2-yl]-carbamic acid ethyl ester (BT2-EOMe). To 2-amino-10-(2-methoxy-ethyl)-10H-dibenzo[b,f][1,4]oxazepin-11-one (2.9 g, 10.2 mmol, 1.0 eq) in 90 ml of DMF under an atmosphere of nitrogen was added diethyl pyrocarbonate (1.82 g, 11.22 mmol, 1.1 eq). The mixture was stirred overnight at 40° C. (external). The solvent was removed and the mixture was dissolved in DCM (200 ml) and the organic phase washed twice with water (150 ml). The organic layer was separated and dried with MgSO₄, filtered on a sintered funnel, and the solvent was removed to give 3.6 g of a brown solid as a crude product. This solid was purified by column chromatography in 30% EtOAc in hexane to give a pure compound 3.5 g (96%) as a colorless solid. ¹H-NMR (400 MHz, CDCl₃) δ=1.30 (t, 3H); 3.40 (s, 3H); 3.80 (t, 2H); 4.20-4.25 (m, 4H); 6.60 (s, 1H); 7.13-7.26 (m, 5H); 7.55-7.60 (m, 2H); 7.68 (s, 1H) ppm.

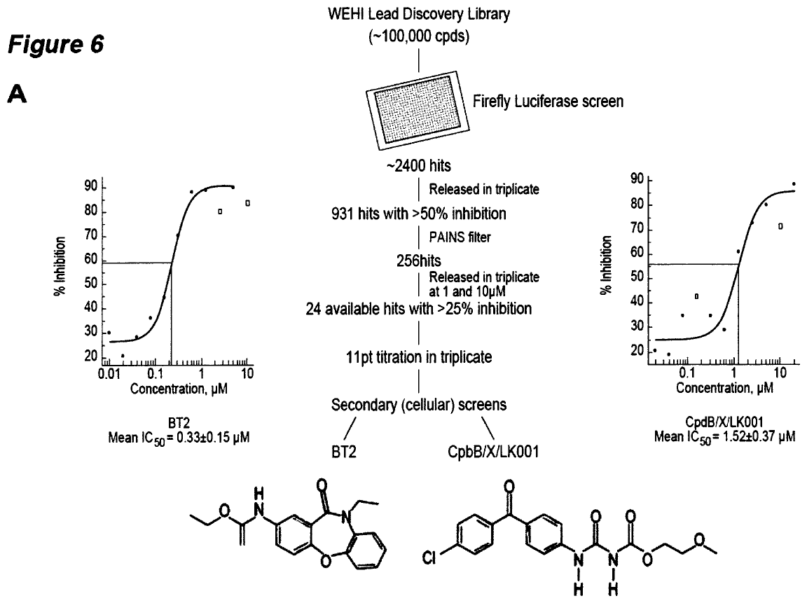

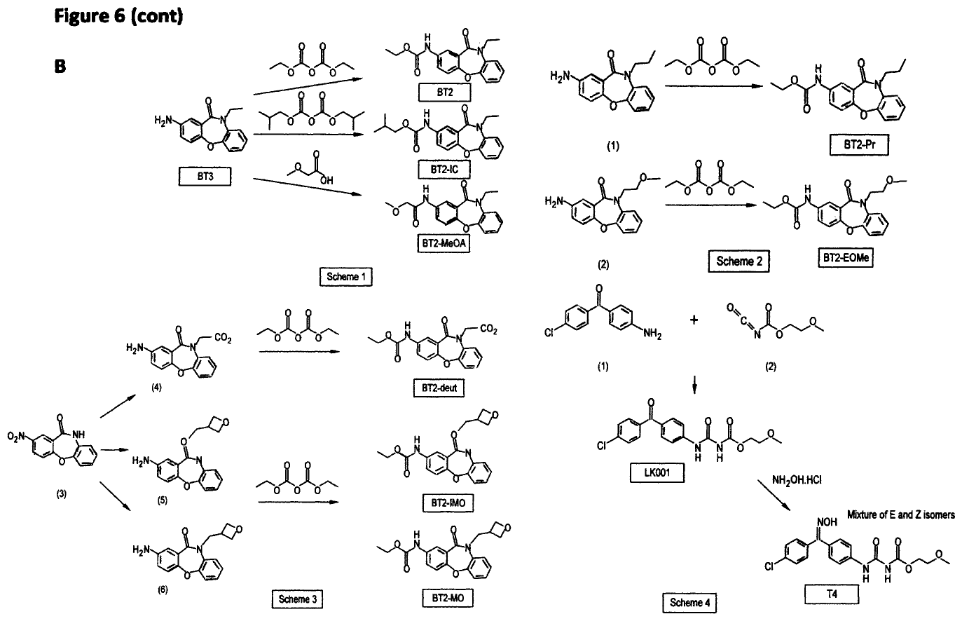

Ethyl (11-(oxetan-3-ylmethyl)dibenzo[b,f][1,4]oxazepin-2-yl)-carbamate and (BT2-IMO) and Ethyl (10-(oxetan-3-ylmethyl)-11-oxo-10,11-dihydro-dibenzo[b,f][1,4]oxazepin-2-yl)-carbamate (BT2-MO). Under an atmosphere of nitrogen 2-nitro-10H-dibenzo[b,f][1,4]oxazepin-11-one (3) (FIG. 6B, Scheme 3) (7.5 g, 0.029 mol, 1.0 eq) was added to 100 ml of DMF and stirred for 5 min. Then, NaH (1.4 g, 2.34 g (60%) in oil, 0.058 mol, 2.0 eq) was added in small portion (an exotherm was observed). The mixture was stirred at 40° C. (external) for 35 min. Then, oxetan-3-ylmethyl methanesulfonate (9.73 g, 0.058 mol, 2.0 eq) was added and the reaction stirred for 3 h at 40° C. (external). The reaction was followed by TLC in 20% ethyl acetate in hexane. When the reaction was complete the solvent was removed on a kugel (100° C. and full vacuum) (or on rotary evaporator, using a strong pump ensuring bath temperature below 70° C.) then water (300 ml) was added. The solid was stirred with a spatula to make it precipitate in water. After filtration the solid was dried in a vacuum oven overnight at 80° C. The crude product mixture was purified by column chromatography on silica gel and a mixture of hexane: ethyl acetate (starting from 20% ethyl acetate in hexane, with polarity increased to 40%). The first band was the O-alkylated compound (RF=0.65).

A 1.0 g (13% yield) of O-alkylated compound yield was obtained as a faint yellow solid. The melting point is 135-137° C. (corrected). ¹H-NMR (400 MHz, D₆-DMSO): δ=3.42-3.57 (m, 1H); 4.53 (t, 2H); 4.65 (d, 2H); 4.74-4.79 (app. dd, 2H); 7.19-7.27 (m, 3H); 7.32-7.36 (m, 1H); 7.62 (d, 1H); 8.38 (d, 1H); 8.47 (dd, 1H) ppm.

Second, O-alkyl. To 2-nitro-11-(oxetan-3-ylmethyl)dibenzo[b,f][1,4]oxazepin-11(10H)-one (1.5 g, 4.6 mmol, 1.0 eq) was added 50 ml MeOH. The mixture was stirred at (external) for 15 min to dissolve all the solids. The reaction mixture was cooled to 22° C. and the flask flushed with nitrogen. 10% Pd/C (200 mg) was added and the compound was hydrogenated under an atmosphere of H₂ at 40° C. (external) for 1 h. The solvent was removed to give 1.2 g (98% yield) of a yellow solid which was used for the next step without further purification (purity≥97%. Melting point: 150-152° C. (corrected). ¹H-NMR (400 MHz, D₆-DMSO): 4.48-4.60 (m, 4H); 4.72-4.78 (app. dd, 2H); 5.2 (s, 2H), 6.71-6.75 (m, 2H); 6.95-6.99 (m, 1H); 7.07-7.20 (m, 4H) ppm.

Last, O-Alkyl. To 2-amino-10-(oxetan-3-ylmethyl)dibenzo[b,f][1,4]oxazepin-11(10H)-one (1.2 g, 4.05 mmol, 1.0 eq) in 40 ml of DMF was added diethyl pyrocarbonate (0.98 g, 6.07 mmol, 1.5 eq). The mixture was stirred overnight at 40° C. (external). The solvent was removed, the mixture was dissolved in DCM (150 ml) and washed twice with water (150 ml). Then, the organic layer was dried with MgSO₄, filtered on a sintered funnel and the solvent was removed to give 1.31 g of a faint yellow solid as a crude product. Crude product (1.3 g) was purified by column chromatography on silica gel using a mixture of hexane:ethyl acetate (starting from 20% ethyl acetate in hexane, then polarity increases to 35%).

BT2-IMO. First, a 0.5 g (34% yield) was obtained as a colorless solid. The melting point is 159-162° C. (corrected). ¹H-NMR (400 MHz, CDCl₃): δ=1.30 (t, 3H); 3.47-3.59 (m, 1H), 4.22 (q, 2H); 4.63-4.68 (m, 4H); 4.88-4.93 (m, 2H); 6.60 (s, 1H); 7.07-7.260 (m, 5 H); 7.50-7.59 (m, 2H) ppm.

A 3.5 g of N-alkylated compound (48% yield) was obtained as a faint yellow solid. (RF=0.45). The melting point is 106-109° C. (corrected). ¹H-NMR (400 MHz, D₆-DMSO) δ=3.17-3.28 (m, 1H); 4.29 (t, 2H); 4.47 (br d, 2H); 4.53-4.58 (app. dd, 2H); 7.26-7.37 (m, 2H); 7.46 (dd, 1H); 7.57-7.64 (m, 2H); 8.40 (dd, 1H); 8.46 (d, 1H) ppm.

Second, N-alkyl. To a 250 ml RBF set up for hydrogenation was added 2-nitro-10-(oxetan-3-ylmethyl)dibenzo[b,f][1,4] oxazepin-11(10H)-one (2.5 g, 6.12 mmol, 1.0 eq) and MeOH. The mixture was stirred at 40° C. (external) for 15 min to dissolve all the solids. The flask was cooled to 22° C. and flushed with nitrogen again. 10% Pd/C (200 mg) was added and the mixture stirrer under an atmosphere of hydrogen at 40° C. (external) for 1 h at atmospheric pressure. The mixture was filtered through celite and the solvent was removed to yield a pure colorless solid (1.8 g, 99% yield) used for the next step without further purification. Melting point: 62-72° C. (corrected). ¹H-NMR (400 MHz, D₆-DMSO) δ=3.11-3.22 (m, 1H); 4.26 (t, 2H); 4.53 (app. dd, 2H); 5.17 (br s, 2H), 7.67 (dd, 1H); 6.85 (d, 1H); 6.95 (d, 1H); 7.17-7.29 (m, 3H); 7.49 (dd, 1H) ppm.

Last, N-alkyl. To 2-amino-10-(oxetan-3-ylmethyl)dibenzo[b,f][1,4]oxazepin-11(100H)-one (1.6 g, 5.49 mmol, 1.0 eq) and diethyl pyrocarbonate (1.44 g, 8.91 mmol, 1.5 eq) was added in 50 ml of DMF. The mixture was stirred for 1 h at 40° C. (external). The solvent was removed, the mixture was dissolved in DCM (150 ml) and washed twice with water (150 ml). The organic layer was dried with MgSO₄, filtered on a sintered funnel and the solvent was removed to give the crude product which was purified by column chromatography using 50% EtOAc in hexane. BT2-MO (1.91 g, 87% yield) was obtained as a colorless solid. Melting point: 161-162° C. (corrected). ¹H-NMR (400 MHz, CDCl₃) δ=1.30 (t, 3H); 3.36-3.48 (m, 1H), 4.20 (q, 2H); 3.80 (t, 2H); 4.31-4.55 (m, 4H); 4.70-4.76 (m, 2H); 6.65 (s, 1H); 7.13-7.26 (m, 5H); 7.57 (d, 1H); 7.70 (s, 1H) ppm.

2-Methoxyethyl[[[4-(4-chlorobenzoyl)phenyl]amino]carbonyl]carbamate (Cpd B/X/LK001). A solution of (4-amino-phenyl)-(4-chloro-phenyl)-methanone (49.1 g, 210 mmol) in DCM (150 ml) was cooled in an ice/NaCl bath to −0° C. (internal temperature). 2-methoxyethyl carbonisocyanatidate (40 g, 276 mmol) in DCM (150 ml) was added via a dropping funnel with the internal temperature being kept below 5° C. The ice bath was removed and the solution stirred for 1 h at 22° C. under nitrogen. The solution was filtered and the solid rinsed with methanol to give a pure faint yellow crop of desired product. A further crop was obtained by concentrating the filtrate (mixture of DCM and MeOH), filtration and methanol wash. Fractions were combined to give 49 g (62%) of the desired product. 1H-NMR (400 MHz, D₆-DMSO) δ=10.52 (s, 1H, NH), 10.10 (s, 1H, NH), 7.75-7.68 (m, 6H), 7.62 (d, 2H), 4.80 (t, 2H), 3.58 (t, 2H), 3.28 (s, 3H) ppm.

2-Methoxyethyl[[[4-(4-chlorophenyl)(hydroxyimino)methyl) phenyl]amino]carbonyl]carbamate (T4). 2-Methoxyethyl[[[4-(4-chlorobenzoyl)phenyl]amino]carbonyl]carbamate (20.7 g, 55 mmol), hydroxylamine hydrochloride (11.4 g, 165 mmol) and sodium acetate (13.5 g, 165 mmol) were stirred under a nitrogen atmosphere at reflux for 4 h. The reaction mixture was filtered hot to remove any salts. The filtrate was cooled and the product filtered. The filtrate was concentrated by two-thirds, cooled to 22° C. and filtered to give a second crop. The solid was vacuum dried at 60° C. to give the desired product (15.8 g, 73%). ¹H-NMR (400 MHz, D₆-DMSO) as a mixture of E and Z isomers (˜1:1) δ=11.43 (s, 0.46H, OH), 11.32 (s, 0.62H, OH), 10.42 (bs, 1H, NH), 9.90 (s, 0.49H, NH), 9.88 (s, 0.63H, NH), 7.59 (d, 0.94H), 7.52 (t, 2.44H), 7.42 (q, 1.92H), 7.35-7.25 (m, 3.44H), 4.28 (m, 2H), 3.58 (m, 2H), 3.27 (s, 1.28H), 3.28 (s, 1.72H) ppm.

Ethyl (10-ethyl(2,2,2′-d3)-11-oxo-10,11-dihydrodibenzo[b,f][1,4]oxazepin-2-yl)carbamate (BT2-deut). First, 2-nitro-10H-dibenzo[b,f][1,4]oxazepin-11-one (1 g, 3.9 mmol, 1 eq) was added to 10 ml of DMF and stirred for 5 min under nitrogen. Then, the NaH (187 mg, 0.32 g in oil, 7.8 mmol, 2 eq) was added in small portions. The mixture was stirred at 40° C. external for 35 min. Then, ethyliodide-2,2,2-d₃ (1.24 g, 0.62 mL, 7.8 mmol, 2 eq) was added and the reaction was stirred for 3 hours at 40° C. external. The solvent was removed via evaporation and trituration three times with water returned a thick paste which was subjected to chromatography eluting using 15% EtOAc in hexane gave 10-(ethyl-2,2,2-d3)-2-nitrodibenzo[b,f][1,4]oxazepin-11(10H)-one as a yellow solid (0.42 g, 38%). The melting point is 142.3° C. to 145.6° C. (corrected). ¹H-NMR (400 MHz, D₆-DMSO): δ=4.12 (app s, 2H), 7.25 to 7.38 (m, 2H), 7.45 (dd, 1H), 7.60 (d and dd, 2H), 8.41 (dd, 1H), and 8.45 (d, 1H) ppm.

Second, 10-(ethyl-2,2,2-d3)-2-nitrodibenzo[b,f][1,4]oxazepin-11(10H)-one (0.4 g, 1.41 mmol, 1 eq) and SnCl2 (0.8 g, 4.2 mmol, 3 eq) were dissolved in 10 ml of EtOH. The mixture was stirred at reflux for 2 hours. The solvent was removed and the mixture was dissolved in EtOAc (100 ml) and 1N NaOH aq. (50 ml). The organic phase was separated and washed with water (2×50 ml), dried with MgSO₄, filtered and the solvent evaporated. Chromatography eluting with 50% EtOAc in hexane gave the product 2-amino-10-(ethyl-2,2,2-d3)dibenzo[b,f][1,4]oxazepin-11(10H)-one as a faint beige solid (287 mg, 80%). The melting point is 165.5° C. to 167.0° C. (corrected). ¹H-NMR (400 MHz, D₆-DMSO): δ=4.0 (bq, 2H), 5.15 (s, 2H), 6.65 (dd, 1H), 6.84 (d, 1H), 6.95 (d, 1H), 7.15 to 7.30 (m, 3H), and 7.45 (dd, 1H) ppm.

Last, to 2-amino-10-(ethyl-2,2,2-d3)dibenzo[b,f][1,4]oxazepin-11(10H)-one (0.287 g, 1.2 mmol, 1 eq) in DMF (3 ml) was added diethyl pyrocarbonate (0.183 ml, 0.201 g, 1.24 mmol, 1.1 eq). The mixture was stirred for 1 hour under nitrogen at 25° C. external. DMF was removed from the reaction mixture and the residual solid was triturated 3 times with EtOAc to give BT2-deut as a colorless solid (260 mg, 66%). The melting point is 184.3° C. to 185.7° C. (corrected). ¹H-NMR (400 MHz, D₆-DMSO): δ=1.22 (t, 3H), 4.00 to 4.15 (q and br q, 4H), 7.2-7.3 (m, 3H), 7.35 (dd, 1H), 7.50 (dd, 1H), 7.58 (dd, 1H), 7.80 (d, 1H), and 9.75 (s, 1H) ppm.

Flubendazole (T6), 2-Amino-10-ethyldibenzo[b,f][1,4] oxazepin-11 (10H)-one (BT3) and (4-Aminophenyl)(4-fluorophenyl)methanone (T7) are available commercially from AK Scientific Inc.

Cell culture. HMEC-1 were obtained from ATCC (Rockville, MD) and grown in MCDB131 medium (Invitrogen, MD), pH 7.4 supplemented with 10% FBS, hydrocortisone (1 μg/ml), epidermal growth factor (10 ng/ml), L-glutamine (2 mM) and penicillin/streptomycin. Bovine aortic endothelial cells (BAEC) were obtained as primary cells from Cell Applications (San Diego, CA) and grown in DMEM, pH 7.4 supplemented with 10% FBS and antibiotics. BAEC were used in experiments between passages 4-6. Cells were routinely passaged after detachment with 0.05% trypsin/5 mM EDTA and maintain in a humidified atmosphere of 5% CO₂ at 37° C.

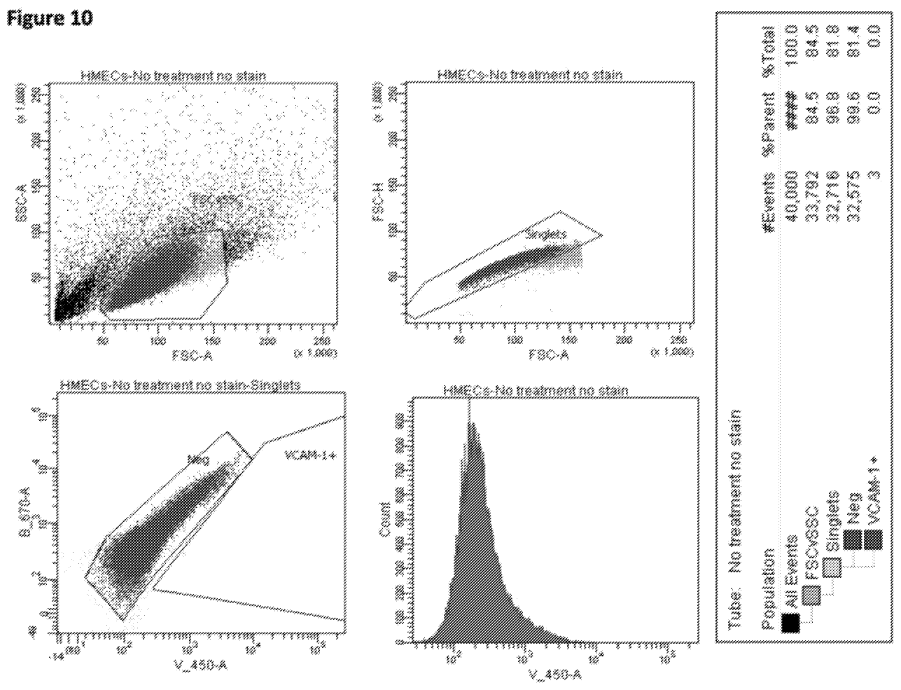

Western blot analysis with extracts of cells treated with serum. HMEC-1 (80-90% confluency) were arrested in serum-free MCDB131 medium without EGF or hydrocortisone for 20 h. Cells were treated with 30 μM compound in serum-free MCDB131 medium for 4 h, and the medium was changed to complete medium (with 10% FBS with EGF and hydrocortisone) with 30 μM compound for 1 h. Total protein was harvested as previously described in radioimmunoprecipitation (RIPA) lysis buffer with protease inhibitors (Li, Y., et al., Int J Cardiol 220, 185-191 (2016)). Proteins were resolved on 4-20% (w/v) sodium dodecyl sulfate (SDS)-polyacrylamide gradient gels (Bio-Rad Mini-PROTEAN TGX) and transferred to Immobilon-P PVDF membranes (Millipore, USA). Membranes were blocked with 5% skim milk and incubated with rabbit monoclonal FosB (cat. 2251, 1:1000, Cell Signaling, USA), rabbit monoclonal c-Fos antibodies (cat. 2250, 1:1000, Cell Signaling, USA) at 4° C. overnight or mouse monoclonal f3-actin antibodies (cat. A5316, 1:30000, Sigma-Aldrich) at 22° C. for 15 min then incubated with a horseradish peroxidase conjugated secondary goat anti-rabbit (cat. P0448, 1:1000, DAKO Cytomation, Denmark) or goat anti-mouse (cat. P0447, 1:1000, DAKO Cytomation, Denmark) antibodies for 1 h. Chemiluminescence was detected using the Western Lightning Chemiluminescence system (PerkinElmer, USA) and ImageQuant™ LAS 4000 biomolecular imager (GE Healthcare Life Sciences, USA). Band intensity in images generated with the LAS 4000 on automatic exposure with sensitivity/resolution setting high was quantified using NIH ImageJ.
Western blot analysis with extracts of cells treated with IL-β. HMEC-1 (80-90% confluency) were arrested in serum-free MCDB131 medium (Invitrogen, MD) without any growth factor for 48 h. Cells were treated with 30 μM compound in serum-free medium for 4 h, and incubated with 20 ng/ml IL-1β (Sigma, cat. SRE3083) in serum-free medium with the same concentration of compound for up to 4 h, unless otherwise indicated. Total protein was harvested as previously described using RIPA buffer with protease inhibitors. Proteins were resolved on 4-20% (w/v) SDS-polyacrylamide gradient gels and transferred to Immobilon-P PVDF membranes. Membranes were blocked with 5% skim milk and incubated with rabbit monoclonal FosB (cat. 2251S, 1:1000, Cell Signaling, USA), rabbit monoclonal VCAM-1 (cat. 13662S, 1:1000, Cell Signaling, USA), rabbit monoclonal p44/42 MAPK (cat. 4695S, 1:1000, Cell Signaling, USA), rabbit polyclonal p38 MAPK (cat. 9212S, 1:1000, Cell Signaling, USA), rabbit polyclonal SAPK/JNK (cat. 9252S, 1:1000, Cell Signaling, USA), rabbit monoclonal phospho-SAPK/JNK (cat. 4671S, 1:1000, Cell Signaling, USA), rabbit monoclonal phospho-p38 MAPK (cat. 4511S, 1:1000, Cell Signaling, USA), or, mouse monoclonal phospho-p44/42 MAPK antibodies (cat. 9106S, 1:2000, Cell Signaling, USA) at 4° C. overnight or mouse monoclonal β-actin antibodies (cat. A5316, 1:10000, Sigma-Aldrich) antibodies at 22° C. for 1 h. Membranes were then incubated with horseradish peroxidase conjugated secondary goat anti-rabbit (cat. P0448, 1:1000, DAKO Cytomation, Denmark) or goat anti-mouse (cat. P0447, 1:1000, DAKO Cytomation, Denmark) antibodies for 1 h. Chemiluminescence was detected using the Western Lightning Chemiluminescence system and ImageQuant™ LAS 4000 biomolecular imager. Band intensity in images generated with the LAS 4000 using the same settings were quantified by NIH ImageJ.
siRNA experiments. HMEC-1 (70-80% confluency) were arrested in serum-free MCDB131 medium with no hydrocortisone or EGF for 24 h and transfected with non-targeting siRNA (cat. D-001810-10-50, Dharmacon, USA) or FosB siRNA (cat. L-010086-01-0020, Dharmacon, USA) or VCAM-1 siRNA (cat. L-013351-00-0020, Dharmacon, USA) and Dharma FECT1 transfection reagent (cat. T-2001-03, Dharmacon, USA) mixed for 24 h. siRNA experiments (with 0.6 μM FosB, 0.6 μM VCAM-1) were performed, shoulder-to-shoulder with non-targeting loading control siRNA at the same concentration. The cells were stimulated with 20 ng/ml IL-1β in serum-free complete MCDB131 medium for a further 2 or 4 h. Total protein was harvested using RIPA buffer with protease inhibitors and resolved on 4-20% (w/v) SDS-polyacrylamide gradient gels and transferred to Immobilon-P PVDF membranes. Membranes were blocked with 5% skim milk and incubated with rabbit monoclonal FosB (cat. 2251S, 1:1000, Cell Signaling, USA), rabbit monoclonal VCAM-1 (cat. 13662S, 1:1000, Cell Signaling, USA) at 4° C. overnight or mouse monoclonal β-actin (cat. A5316, 1:10000, Sigma-Aldrich) antibodies at 22° C. for 1 h. Membranes were incubated with horseradish peroxidase conjugated secondary goat anti-rabbit (cat. P0448, 1:1000, DAKO Cytomation, Denmark) or goat anti-mouse (cat. P0447, 1:1000, DAKO Cytomation, Denmark) Ig for 1 h. Chemiluminescence was detected using the Western Lightning Chemiluminescence system and ImageQuant™ LAS 4000 biomolecular imager.
Plasmid overexpression. HMEC-1 were seeded into 6-well plates and at 70-80% confluency, the cells were deprived of serum (or EGF and hydrocortisone) overnight. Cells were transfected with 6 μg of the indicated plasmid (in pcDNA3.1+/C-(K)DYK) (GenScript, USA) with Fugene 6 (Promega) according to manufacturer's protocol. Total protein lysates were collected 18, 24, 48 and 72 h after plasmid transfection in RIPA buffer with protease inhibitors. Proteins were resolved on 4-20% (w/v) SDS-polyacrylamide gradient gels and transferred to Immobilon-P PVDF membranes. Membranes were blocked with 5% skim milk and incubated with rabbit monoclonal p44/42 MAPK (cat. 4695S, 1:1000, Cell Signaling), mouse monoclonal phospho-p44/42 MAPK antibodies (cat. 9106S, 1:2000, Cell Signaling), rabbit monoclonal FosB (cat. 2251S, 1:1000, Cell Signaling, USA), rabbit monoclonal VCAM-1 (cat. 13662S, 1:1000, Cell Signaling) or mouse monoclonal α-tubulin (cat. T5168, 1:40000, Sigma) at 4° C. overnight. Membranes were then incubated with horseradish peroxidase conjugated secondary goat anti-rabbit (cat. P0448, 1:1000, DAKO Cytomation, Denmark) or goat anti-mouse (cat. P0447, 1:1000, DAKO Cytomation, Denmark) antibodies for 1 h. Chemiluminescence was detected using the Western Lightning Chemiluminescence system and ImageQuant™ LAS 4000 biomolecular imager.
RNA-seq. HMEC-1 were seeded into nine 100 mm petri dishes with complete MCDB131 medium containing 10% FBS. At 70-80% confluency, cells were growth-arrested with serum-free MCDB131 medium with no hydrocortisone or EGF for 44 h. Cells were pre-treated with 30 μM BT2 in the same medium for 4 h then stimulated with 20 ng/mL IL-1β for a further 4 h. Total RNA was extracted using RNeasy Mini Kit (Qiagen, Amtsgericht Düsseldorf) with modification. Briefly, cells were washed twice with pre-cooled 1× PBS and TRIzol (Thermo Fisher Sci, Waltham, MA) was added to lyze the cells. Chloroform was added to the mixture prior to centrifugation at 13000 rpm for 15 min at 4° C. Upper aqueous layer containing total RNA was transferred to fresh microtubes and isopropanol was added and loaded into RNeasy column. Columns were washed with Buffers RW1 and RPE. Total RNA was eluted from the column using RNAse-free water. Samples were submitted to The Ramaciotti Centre for Genomics (UNSW, Australia) for TruSeq Stranded mRNA-seq preparation and sequencing by One NextSeq 500 1X75 bp High Output flowcell with data output up to 400M reads. Quality control of samples was set at >80% higher than Q30 at 1×75 bp.
RNA-seq reads were first assessed for quality using the tool FastQC (v0.11.8) (On the World-Wide-Web at: bioinformatics.babraham.ac.uk/projects/fastqc/). The tool Salmon was used for quantifying transcript abundance from RNA-seq reads (Patro, R., et al., Nat Methods 14, 417-419 (2017)). The R package DESeq2 (Love, M. I., et al., Genome Biol 15, 550 (2014)) that incorporates a method for differential analysis of count data was then used to identify differentially expressed genes across specific comparisons. The heatmap.2 function from the R package gplots v3.0.1.1 was used to generate heatmaps using counts per million (cpm) values for sets of genes of interest. The database for annotation, visualization and integrated discovery (DAVID) (Jiao, X., et al., Bioinformatics 28, 1805-1806 (2012)), a web-based online bioinformatics resource was used to identify the gene ontologies such as biological processes (BP) found to be enriched for lists of differentially expressed genes for specific comparisons.
Flow cytometry. HMEC-1 (at 80-90% confluency) were arrested in serum-free MCDB131 medium without EGF or hydrocortisone for 40 h, treated with 30 μM BT2 or BT3 for 4 h. The cells were incubated in serum-free medium and exposed to 20 ng/ml IL-1β with the same concentration of BT2 or BT3 for a further 4 h. The cells were washed with PBS then detached with Accutase (Stem Cell Technologies, cat. 07920). The cells were centrifuged at 300 g for 5 min and resuspended at 5×10⁶ cells/ml containing BT2 or BT3. The cells were incubated with BV421-conjugated mouse anti-human CD106 (VCAM-1) (BD, cat. 744309) or BV421-conjugated mouse IgGi (BD, cat. 562438) for 45 min at 22° C. The cells were washed with Stain Buffer and the pellet was resuspended in 0.5 ml of 1% paraformaldehyde prior to flow cytometry BD FACSCanto II.
VCAM-1⁺ and VCAM-1⁻ cells were gated by performing flow cytometry with or without primary VCAM-1 antibody (non-specific staining), respectively. Representative gating from the latter (i.e. negative control) is shown as FIG. 10 indicate minimal non-specific staining. The gating strategy is based on fluorescence excitation off both the 488 nm laser and 405 nm laser with emission filters 670LP off 488 nm and 450/50 off 405 nm. Cells with autofluorescence or negative (blue population) had equal proportion of fluorescence in both channels and VCAM-1 positive cells (red) emit light in the 450/50 filter.
SPR. SPR was performed on a Biacore T200. The active and reference flow cells of a Xantec NIHMC Ni sensor chip were conditioned with 0.5M NaEDTA followed by 5 mM NiCl₂ in immobilisation buffer (20 mM HEPES, 150 mM NaCl, pH 7.4). Recombinant human His-MEK1 and His-MEK2 (500 nM, ThermoFisher Scientific, cat. PV3303 and PV3615, respectively) were injected for 15 min at 100 min⁻¹ over separate active flow cells. All immobilisation was carried out at 25° C. Following immobilisation, the temperature was lowered to 15° C., and the buffer changed to 20 mM HEPES, 150 mM NaCl, 5% DMSO pH 7.4. Samples of PD98059 (2.5-30 μM in running buffer) and BT2 (1.25-15 μM) were injected at a flow rate of 300 min⁻¹ over immobilised MEK1 and MEK2. Solvent correction was applied to the data using a DMSO standard curve. Data were analysed using the Biacore T200 Evaluation software. Prior to SPR, limits of compound solubility were determined using ¹H 1D NMR.
Endothelial proliferation assay using the xCELLigence system. HMEC-1 proliferation was evaluated using the xCELLigence System (Roche, Castle Hill). Briefly, HMEC-1 (5×10³ cells/well) were seeded in a 96-well E-plate and inserted into the xCELLigence RICA station (Roche). Cells were serum-deprived for 24 h in MCDB131 medium which contained 10 ng/ml EGF (Sigma-Aldrich) and 1 μg/ml hydrocortisone (Sigma-Aldrich) then treated with compound (0.2-1 μM) in medium containing 5% FBS, 10 ng/ml EGF (Sigma-Aldrich) and 1 μg/ml hydrocortisone (Sigma-Aldrich). Cell growth was monitored automatically every 15 min by xCELLigence system. Cell index (CI) represents a quantitative measure of each well cell growth. In this system, CI a unitless parameter that reports impedance of electron flow caused by adherent cells.
Endothelial proliferation assay using the Countess system. HMEC-1 proliferation was assessed using a Countess II Automated Cell Counter (ThermoFisher Scientific). Briefly, HMEC-1 (3×10⁵ cells/well) were seeded in a 12-well plate. Cells were serum-deprived for 24 h in MCDB131 medium which contained 10 ng/ml EGF and 1 μg/ml hydrocortisone then treated with compound (0.1-0.6 μM) in medium containing 5% FBS, 10 ng/ml EGF and 1 μg/ml hydrocortisone. The cells were trypsinized after 24 h, resuspended in complete medium, a 10 μl aliquot was combined with an equal volume of 4% Trypan Blue, and total cell numbers and Trypan Blue-excluding cells as a proportion of total was determined using the Countess.
Endothelial dual chamber migration assay. BAEC (6×10³ cells/well) suspended in DMEM supplemented with 10% FBS were seeded into the upper chamber of 24-well plates fitted with Millicell cell culture inserts (cat. P18P01250, Millipore). After 48 h, the medium was changed to DMEM supplemented with 0.01% FBS and the cells were incubated for 48 h. Compounds prepared in DMEM containing 0.01% FBS were added to the upper chamber. VEGF-A₁₆₅ (50 ng/ml, Sigma, cat. V7259) in medium containing 10% FBS was added to the lower chamber. After 24 h, medium from the upper chamber was removed and a cotton swab was used to remove non-migrated cells and excess liquid. The insert was placed in 70% ethanol for 10 min to allow cell fixation and membranes were dried for 10-15 min. Filters were excised and placed on slides. Mounting medium (Fluoroshield™ with DAPI, Sigma, cat. 6057) was added and specimens were visualized using an EVOS FL microscope.

Endothelial repair following in vitro injury. HMEC-1 (90-100% confluency) in 6-well plates were washed with PBS, and treated with 0.6 μM compound in MCDB131 containing 5% FBS. A sterile pointed toothpick was used to scrape the cell monolayer and the wells photographed under 4× objective at 0 h and 48 h. Cell regrowth in the denuded zone was determined using Image-Pro Plus (Cybernetics, USA).

BT2 formulation analysis using RRLC-MS/MS. A rapid resolution liquid chromatography/tandem mass spectrometry (RRLC-MS/MS) method was developed under GLP by Iris Pharma using an Agilent 1200 Triple Quad G6410B to determine BT2 content in heat-treated or non-heat treated BT2 formulations at 1 week (T1 week) or 6 weeks (T6 weeks) after preparation at room temperature. The formulations were heat (H)-treated (tubes placed in a 100° C. water bath for 10 min) or non-heat treated sonicated formulations of BT2 in saline containing 0.5% Tween 80 and 0.01% DMSO). Standard curves were constructed with 8 concentrations between the lower limit of quantification (LLOQ) and the upper limit of quantification (ULOQ). Evaluations were performed on 3 preparations at the same dilution. Chromatograms were integrated using MassHunter software. For BT2 content analysis (T1 week and T6 weeks), calculation of mean, SD, CV (%) and bias (%) were performed as follows: For T1, the theoretical concentration (i.e. the weighed/formulated material supplied) was used as reference to calculate the bias (%) of each preparation containing the test sample:

$$\% bias = \frac{Calculated value - Theoretical Concentration}{Theoretical Concentration} \times 100.$$

Standard curves were fitted using Excel® version 2011. For each run, bias on back-calculated concentration of the standard curve and QC was determined, with back-calculated concentrations of the calibration standards being set within ±15% of the theoretical value, except for the LLOQ for which it was set within ±20%. At least 75% of the calibration standards, with a minimum of six, must have had to fulfil this criterion and the coefficient of determination (r²) was set at 0.98.
BT2 formulation analysis using liquid chromatography mass spectrometry (LC/MS). DMSO (100 μl) and samples (˜50 μl) were combined along with formic acid (10). These solutions (10 μl) were further diluted with H₂O:CH₃CN (1:1) 0.1% formic acid (90 μl) for LC/MS analysis. Samples were separated by UPLC using an HPG-3400RS UPLC pump, autosampler and column compartment system (Thermo Scientific, CA). Samples (0.1 μl) were loaded onto a Hypersil Gold aQ column (2.1×50 mm) containing 1.9μ media (Thermo Scientific). Compounds were eluted using a linear gradient of H₂O:CH₃CN with A containing H₂O (0.1% formic acid) and B containing H₂O:CH₃CN (1:4, 0.1% formic acid). The gradient was: T=Omin 2% B, T=20 min 75% B, T=23 min 95% B, T=25 min, 95% B, T=25.2 min 2% B, T=30 min, 2% at 200 μl/min over 30 min. The column oven was heated to Positive ions were generated by electrospray and the QExactive Plus mass spectrometer (Thermo Fisher, Bremen, Germany) operated in data dependent acquisition mode (DDA). The heated electrospray source (HESI) was used with a high voltage 3.8 kV applied; a vaporizer temp of 250° C.; sheath gas 20; aux gas 5 and the heated capillary set to T=290° C. A survey scan m/z 140-800 was acquired (resolution=70,000 at m/z 200, with an AGC target value of 3×10⁶ ions, max IT 250 msec) with lockmass was enabled (m/z 391.28429). Up to the 10 most abundant ions combining 2 microscans (with a minimum AGC target of 5×10⁴, maximum IT 110 msec) were sequentially isolated (width m/z 1.8) and fragmented by HCD (NCE=20, 30, 50) with an AGC target of 2×10⁶ ions (resolution=17,500 at m/z 200). M/Z ratios selected for MS/MS were dynamically excluded for 12 sec and charge state exclusion was not enabled. LC/MS chromatograms were processed using Xcalibur Qual Browser.
Endothelial network formation assay. HMEC-1 (4×10⁴ cells/well) in MCDB131 containing 1% FBS and compound (1 or 3 μM) or curcumin (1-40 μM) and 50 ng/ml FGF-2 were added to 96-well plates coated overnight at 4° C. with 1000 of growth factor-reduced reconstituted basement membrane matrix (Matrigel, cat. 354230, Corning, NY). Network formation was observed over subsequent hours and photographed under 4× or 10× objective using an Olympus CKX41 microscope.
Matrigel plug assay. Matrigel (5000) containing VEGF-A₁₆₅ (100 ng/ml), heparin (10 U) and BT2 or BT3 (2.5 mg/mouse) or its vehicle (saline containing 0.01% DMSO and 0.5% Tween 80) was injected subcutaneously into the left flanks of male 8 week-old C57BL/6 mice. After 7 d the mice were sacrificed by CO₂ asphyxiation and the plugs carefully removed. Formalin-fixed paraffin embedded sections were prepared from Matrigel plugs for immunohistological assessment. Heat-induced epitope retrieval was applied to all deparaffinized sections (4 μm Superfrost slides) in citrate buffer, pH 6 for 5 min at 110° C. Immunostaining for all groups with a given antibody was performed simultaneously and development time was identical. Animal experiments were approved by the Animal Care and Ethics Committee at the University of New South Wales.
For CD31 staining, sections were blocked with endogenous enzyme blocking agent (cat. S2003, DAKO) for 10 min and then with 2% skim milk for 20 min. Slides were incubated with primary antibody rabbit polyclonal CD31 antibody, 1:25 dilution (cat. ab28364, Abcam) for 1 h at room temperature. Slides were rinsed with buffer and incubated with secondary antibody (goat anti-rabbit (cat. P0448, DAKO)) for 30 min, rinsed with buffer and incubated with diaminobenzidine (DAB) chromagen (cat. K3468, DAKO) for 5 min and counterstained in hematoxylin and Scott blue. Slides were dehydrated in 100% ethanol and xylene then coverslipped.
For FosB or VCAM-1 staining, sections were blocked with dual endogenous enzyme blocking agent (cat. S2003, DAKO) for 10 min and then with 2% skim milk for 20 min. The slides were incubated with primary rabbit monoclonal FosB (cat. 2251, Cell Signaling, USA) or rabbit polyclonal VCAM-1 (cat. sc-8304, Santa Cruz) for 1 h at room temperature and then incubated for 10 min with the probe component of MACH3 Rabbit AP-Polymer Detection (Biocare Medical, M3R533 G, H, L). After rinsing with buffer the slides were incubated with polymer component of MACH3 Rabbit AP-Polymer Detection (Biocare Medical, M3R533 G, H, L) for a further 10 min. The slides were incubated with red chromogen (Warp Red™ Chromogen Kit) for 7 min and counterstained in hematoxylin and Scott blue. The slides were dried using filter paper and dehydrated in xylene then coverslipped.

Slides were scanned using an Aperio ScanScope XT slide scanner (Leica Biosystems, Mt Waverley, Vic Australia) and images captured using ImageScope software (Leica Biosystems). Positive intraplug staining was assessed using Image-Pro Plus software (Cybernetics, Bethesda, MD) in 5-12 randomly selected fields of view for each plug photographed under 10× (CD31), 20× (VCAM-1) and 40× (FosB) objectives and expressed as integrated optical density (IOD, the product of calibrated intensity (optical density) and area, i.e. IOD=intensity (mean)×area) (Media Cybernetics) (Liu, H., et al., Sci Rep 6, 21319 (2016)). We also expressed positive immunostaining as the area of positive staining as a proportion (%) of plug area (Kim, J. Y., et al., Biomolecules 10, pii: E11 (2019)).

Rabbit retinal vascular hyperpermeability model. Male HY79b pigmented rabbits (8-12 week-old) were anesthetized by an intramuscular injection of Rompun® (xylazine)/Imalgene® (ketamine). Compound (600 μg BT2, BT3 or saline vehicle containing 0.5% Tween 80 and 10% DMSO vehicle in 1000) was injected into the right eye 5 d prior to rhVEGF-A₁₆₅ induction. Injections were performed on anesthetized animals under an operating microscope using a 2500 Hamilton syringe (fitted with 30 G needle). Retinal vascular permeability was induced by a single 500 IVT injection of 500 ng rhVEGF-A₁₆₅ (diluted in PBS with carrier protein) into the right eye. Forty-seven hours (+/−3 h) after induction, sodium fluorescein (10% in saline, 50 mg/kg) was injected into the marginal ear vein. One hour after fluorescein injection, animals were anaesthetized and pupils were dilated by instillation of one drop of 0.5% tropicamide. Ocular fluorescence in both eyes was measured with a FM-2 Fluorotron Master ocular fluorophotometer. Animals were euthanized by injection of pentobarbital. The study was performed by Iris Pharma (La Gaude, France) with approval from the Animal Ethics Committee of Iris Pharma and the Animal Care and Ethics Committee at the University of New South Wales.

Rat choroidal laser injury model. Male Brown Norway pigmented rats (8-14 week-old) were anesthetized by an intramuscular injection of Rompun® (xylazine)/Imalgene® (ketamine). Pupils were dilated by instillation of one drop of 0.5% tropicamide before laser burn. Six burns were created in both eyes on Day 0 by applying 170 mW of 532 nm laser light (Viridis laser, Quantel, France) on 75 μm spots around the optic nerve, between the main retinal vessel branches, for 0.1 s, through the slit lamp and contact lens. Production of a bubble at the time of laser application confirmed the rupture of Bruch's membrane. Compounds in vehicle (saline containing 0.01% DMSO and 0.5% Tween 80, sonicated) in 2-5 μl were injected IVT on Days 0 and 7 under an operating microscope using a 30 G needle mounted on a 1000 Hamilton syringe. Kenacort was administered IVT into each eye on Day 0. Alternatively, aflibercept/Eylea in vehicle (saline) was injected IVT 6 times (Days 0, 3, 7, 10, 14, 17). Fluorescein angiography was performed using Heidelberg retinal angiography. After anaesthesia, 10% sodium fluorescein (250 μl/100 g body weight) was injected subcutaneously, and ocular fluorescence was recorded 10 min after dye injection. Fluorescein leakage was evaluated on Days 14 and 21 in the angiograms by two examiners masked to the study groups and graded for fluorescein intensity as follows: score 0: no leakage; 1: slightly stained; 2: moderately stained; 3: strongly stained. The studies were performed by Iris Pharma (La Gaude, France) with approval from the Animal Ethics Committee of Iris Pharma and the Animal Care and Ethics Committee at the University of New South Wales.

Immunohistochemical staining of rat retina. Rabbit monoclonal anti-CD31 (cat. ab182981), rabbit monoclonal anti-VCAM-1 (cat. ab134047) and rabbit polyclonal anti-VEGF-A (cat. ab46154) were obtained from Abcam. Rabbit monoclonal phospho-p44/42 MAPK (pERK1/2, Thr²⁰²/Tyr²⁰⁴) (cat. 4370) and rabbit monoclonal FosB (cat. 2251) were obtained from Cell Signaling. Formalin-fixed, paraffin embedded sections were prepared from resected rat eyes. Heat-induced epitope retrieval was applied to all deparaffinized sections (4 μm Superfrost slides) with either citrate buffer, pH 6 (VEGF-A, pERK, VCAM-1) or EDTA buffer, pH 9 (CD31) for 5 min at 110° C. Sections were blocked with dual endogenous enzyme blocking agent (cat. S2003, DAKO) for 10 min and then with 2% skim milk for 20 min. Slides were incubated with primary antibody for 60 min at room temperature and then for 10 min with the probe component of MACH3 Rabbit AP-Polymer Detection (Biocare Medical, cat. M3R533 G, H, L). After rinsing with buffer, the slides were incubated with the polymer component of MACH3 Rabbit AP-Polymer Detection (Biocare Medical, M3R533 G, H, L) for a further 10 min. Slides were incubated with red chromogen (Warp Red™ Chromogen Kit) for 7 min and counterstained in hematoxylin and Scott blue. Slides were dried with filter paper and dehydrated in xylene then coverslipped. Immunostaining with a given antibody was performed for all groups at the same time. Immunostained slides were scanned using an Aperio ScanScope XT slide scanner (Leica Biosystems, Mt Waverley, Vic, Australia) and images were captured using ImageScope software (Leica Biosystems). IOD of positive staining (red chromogen) was assessed for CD31, VEGF-A₁₆₅, pERK, FosB and VCAM-1 using Image-Pro Plus software (Cybernetics, Bethesda, MD). IOD in IPL and INL was quantified for CD31; OPL to OS for VEGF-A₁₆₅; INL to ONL for pERK; GCL to OS for FosB; OLM for VCAM-1, using Image-Pro Plus. In addition, we expressed positive immunostaining as area of positive staining relative to retinal tissue area (%) (Kim, J. Y., et al., Biomolecules 10, pii E11 (2019)). On image selection for quantification, for the vehicle and BT2 groups, all wounds in 2-4 sections/eye were identified and photographed under 20× objective. In the untreated group, which had no injury, photographs of 1-3 sections/eye were taken under 20× objective. Staining was quantified with n=3-6 per group. Where VEGF-A₁₆₅ gradient staining was assessed relative to the wound, immunostaining was assessed in 10 consecutive 100 μm boxes starting 150 μm (double headed arrow) from wound center with IOD in each box quantified with Image-Pro Plus.

Endothelial-monocytic cell adhesion assay. HMEC (80-90% confluency) in 96-well plates were deprived of serum for 24 h and treated with compound at indicated concentrations for 1 h then incubated with IL-1β (20 ng/ml) for 4 h. Meanwhile THP-1 were labeled with 5 μM calcein (5×10⁶ cells/ml, BD Bioscience) for 30 min at 37° C. followed by washing 3 times with PBS. THP-1 (2.5×10⁵ cells/well) were then added for 30 min, unbound cells were washed off 3 times in PBS. Adhesion of calcein-labeled THP-1 to the endothelium layer was determined in a fluorescent plate reader at excitation 485 nm and emission 530 nm.

Monocyte-transendothelial migration assay. Millicell 8 μm polycarbonated culture plate inserts (Millipore) were coated with 0.1% porcine gelatin type A (Sigma) and then placed into 24-well plates. HMEC (5×10⁴ cells/well) were seeded onto the insert and allowed to adhere overnight. Cells were then serum deprived for 24 h and treated with various compound treatments for 1 h. IL-1β (20 ng/ml) was added to stimulate the cells for 4 h and 5000 of serum-free medium was added to the bottom of the 24-well plate along with the compound. THP-1 (5×10⁵ cells in 1000) were added into the insert and 100 ng/ml MCP-1 (Sigma) was added to the lower well. After 24 h, the number of cells that had migrated though the endothelial layer was assessed by counting 1000 of the suspension in the lower chamber using a Coulter cell counter (Beckman Coulter).

Collagen antibody-induced arthritis. Arthritis was induced in female Balb/c mice (6-8 week-old) as previously described with a commercially obtained cocktail of 5 monoclonal antibodies to type II collagen at 2 mg/mouse (Chondrex, Inc. Redmond, WA) followed by LPS (50 μg/mouse) with or without BT2 (3 or 30 mg/kg mouse) in DMSO vehicle was administered i.p. on Day 3. Hind footpad thickness was measured on Day 9 using digital calipers. Mice were sacrificed on Day 14 and microCT scanning of hind limbs was performed. Animal experiments were approved by the Animal Care and Ethics Committee at the University of New South Wales.

Micro-CT scanning and analysis. Formalin-ethanol fixed hind limbs were micro-CT scanned prior to histology processing using a Siemens Inveon micro-CT scanner (Victoria, Australia). Data was acquired with the Inveon Acquisition Workplace at 16.84 μm pixel size, 360 projections, 4100 ms integration time, 80 keV photon energy and 140 μA current. 3D models were visualized and snap shots of the limbs acquired with the Inveon Research Workplace software. Data was quantified by binary score where 0=no bone destruction and 1=destruction was given to each individual limb.

Tartrate-resistant acid phosphatase (TRAP) staining. Osteoclasts were stained using TRAP kit (Cosmo Bio, Japan, cat. PMC-AK04F-COS). Sections were heated at 65° C. for 1 h prior dewaxing. Tissue sections were deparaffinised with 100% xylene and rehydrated with 100, 70 and 30% ethanol and rinsed with distilled water for 5 min. Sections were covered with TRAP staining solution containing 3 mg tartaric acid per 50 ml tartaric acid buffer. The sections were incubated at 37° C. for 1 h, then rinsed in distilled water 3 times to halt the reaction. Sections were counterstained with hematoxylin for 5 s then washed in running water until clear then dried. Sections were dehydrated with xylene and air-dried then mounted with aqueous permanent mounting medium. Within the synovium on the medial aspect of each animal joint, 6 random areas photographed under 20× objective were selected in the blinded fashion. Numbers of osteoclasts were counted using NIH Image J. Alternatively TRAP staining was quantitated using IOD (Image-Pro Plus).

Immmunohistochemical staining of hind limbs for VCAM-1 and ICAM-1 and analysis. Formalin-fixed, paraffin embedded of hind limbs were sectioned (5 μm). Dako EnVision Rabbit Kit (cat. K4011, Dako) was used for immmunohistochemical staining for VCAM-1 and ICAM-1. Briefly, sections were blocked with peroxidase for 30 min and then immunostained with rabbit monoclonal VCAM-1 (cat. ab134047, 1:100, Abcam), or rabbit polyclonal ICAM-1 (cat. ab124759, 1:100, Abcam) at 4° C. for overnight. Staining was visualized using labeled polymer-Horse Radish Peroxidase (HRP) (anti-rabbit) and

Diaminobenzidine (DAB) system followed by counter staining with haematoxylin and Scott blue. Immunostained slides were scanned using an Aperio ScanScope XT slide scanner (Leica Biosystems, Mt Waverley, Vic, Australia) and images were captured using ImageScope software (Leica Biosystems). Integrated optical density (IOD) of positive staining in ankle joint (tibia and talus) articular cartilage was assessed for VCAM-1 and ICAM-1 using Image-Pro Plus software (Cybernetics, Bethesda, MD, USA). Area (μm²) of ankle joint articular cartilage was measured using Image-Pro Plus software. Total cell number and positive staining cell number in ankle joint articular cartilage were counted manually using Image-Pro Plus software. Data was represented as IOD/μm 2 and percentage of positive staining cell per 20× objective view.

Toxicology. Female Balb/c mice (8-9 week-old) were given 3 or 30 mg/kg BT2 (DMSO vehicle) via intraperitoneal injection (Days 0 and 5 in DMSO), oral gavage (Days 0-4 in DMSO/methylcellulose) or intraarticular injection (Day 0 in DMSO). Mice were euthanized after 8-11 d. Tissues was fixed in 10% formalin, processed routinely, sectioned at 4 μm and stained with hematoxylin and eosin. Sections were examined histologically for signs of toxicity by a board-certified diplomate of the American College of Veterinary Pathologists. Animal experiments were approved by the Animal Care and Ethics Committee at the University of New South Wales.
Statistics. Statistical analysis was performed as described in the legends using PRISM v7.0d and differences were considered significant when P<0.05. Where indicated, *P<0.05, **P<0.01, ***P<0.001, ****P<0.0001.

### Results

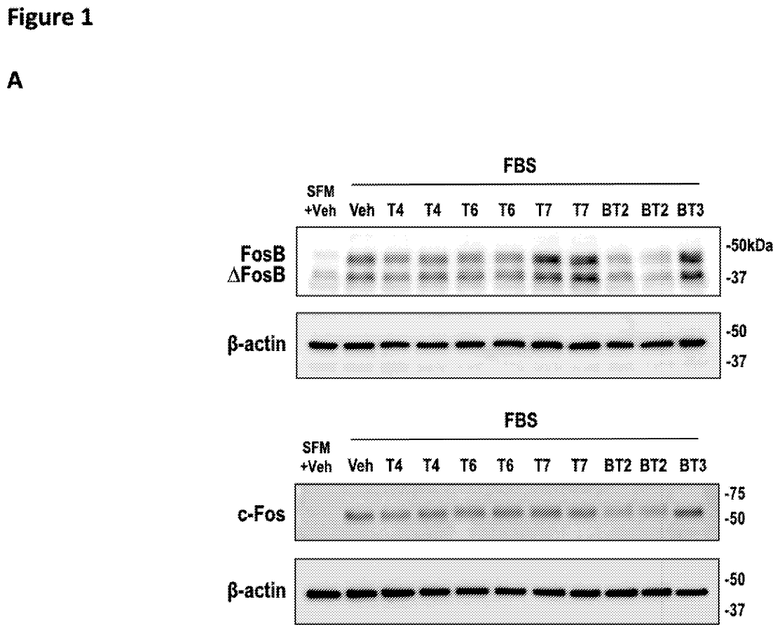

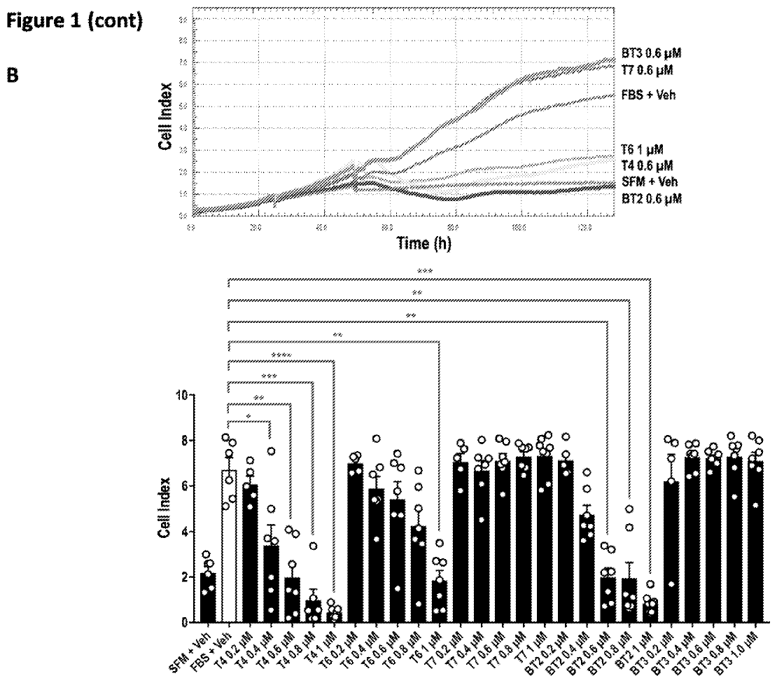

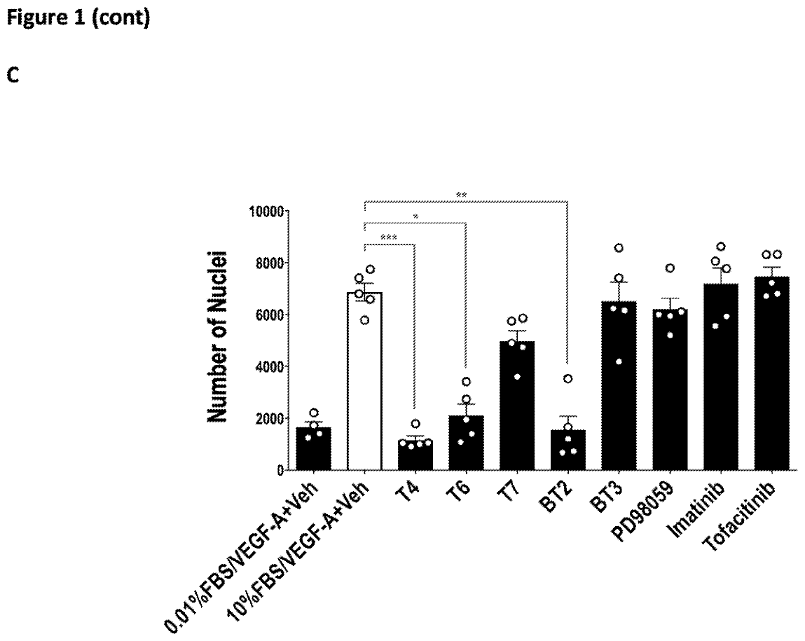

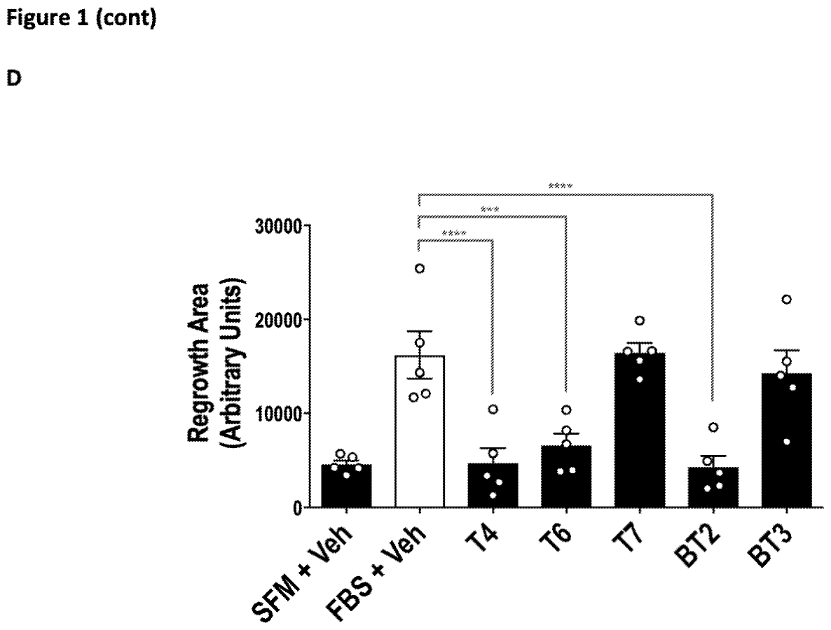

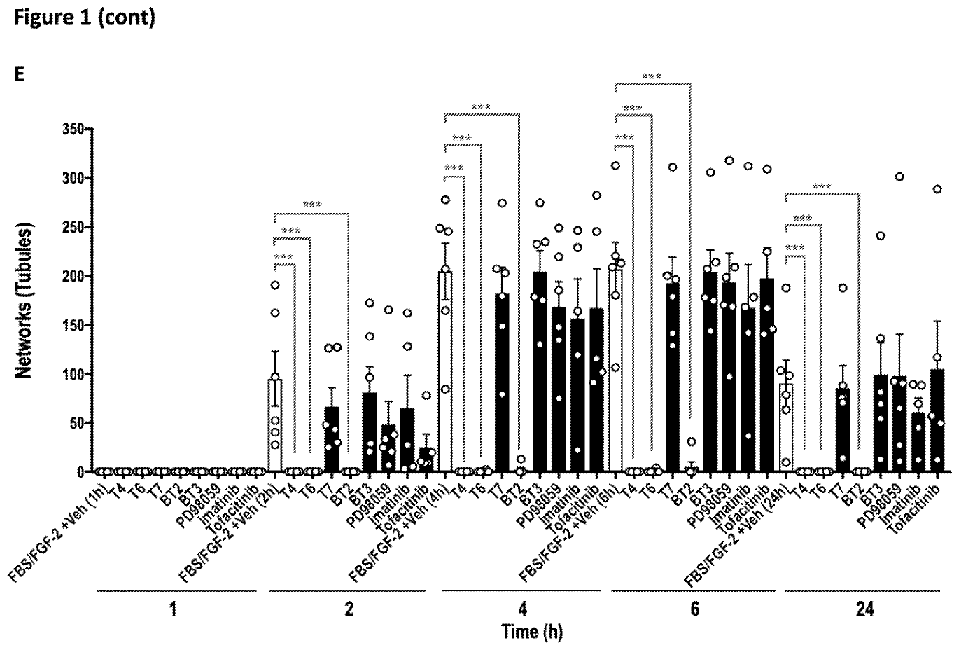

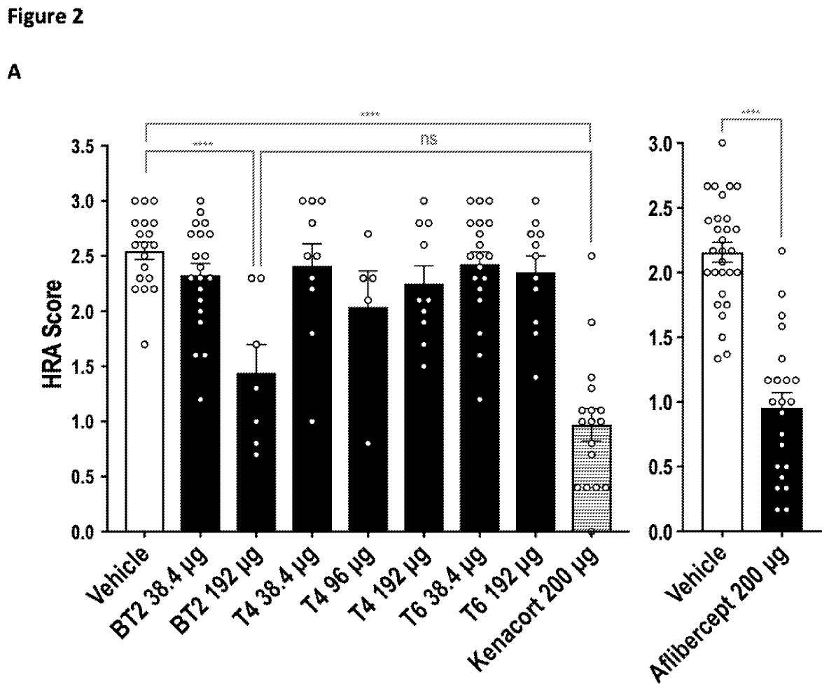

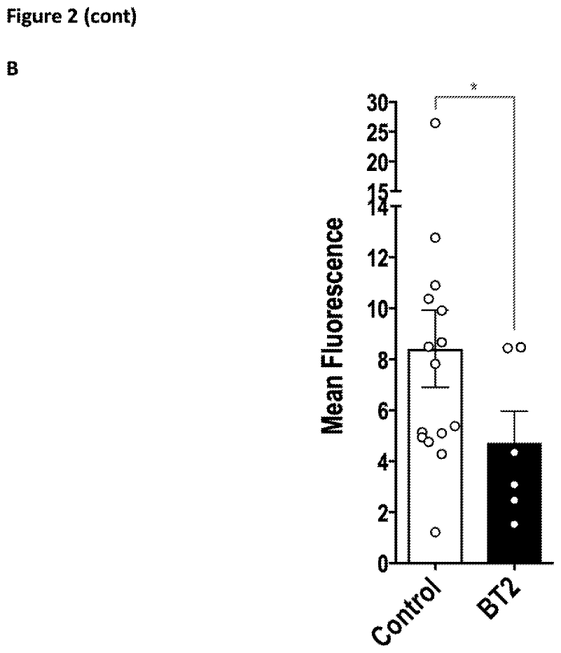

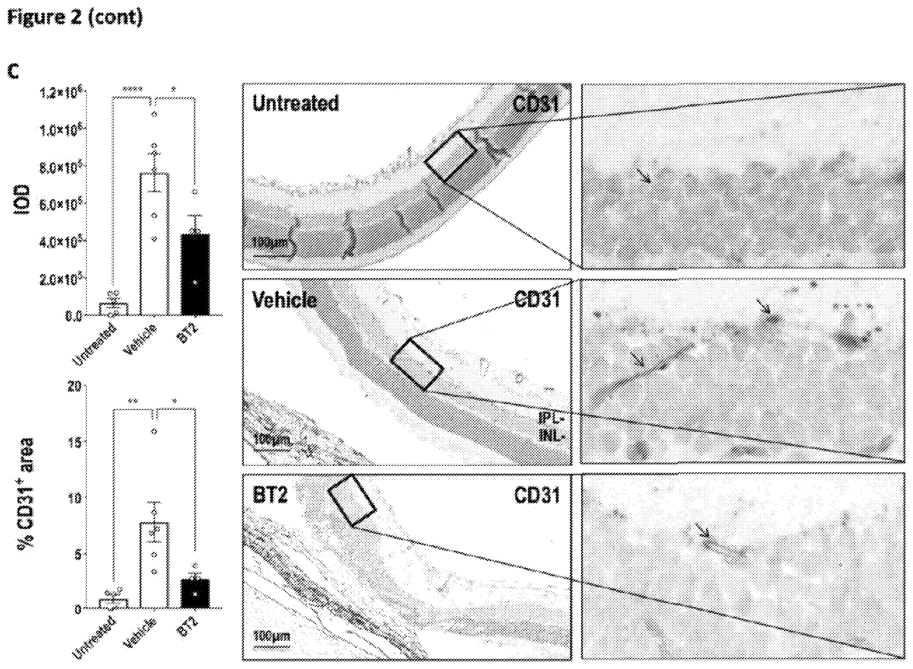

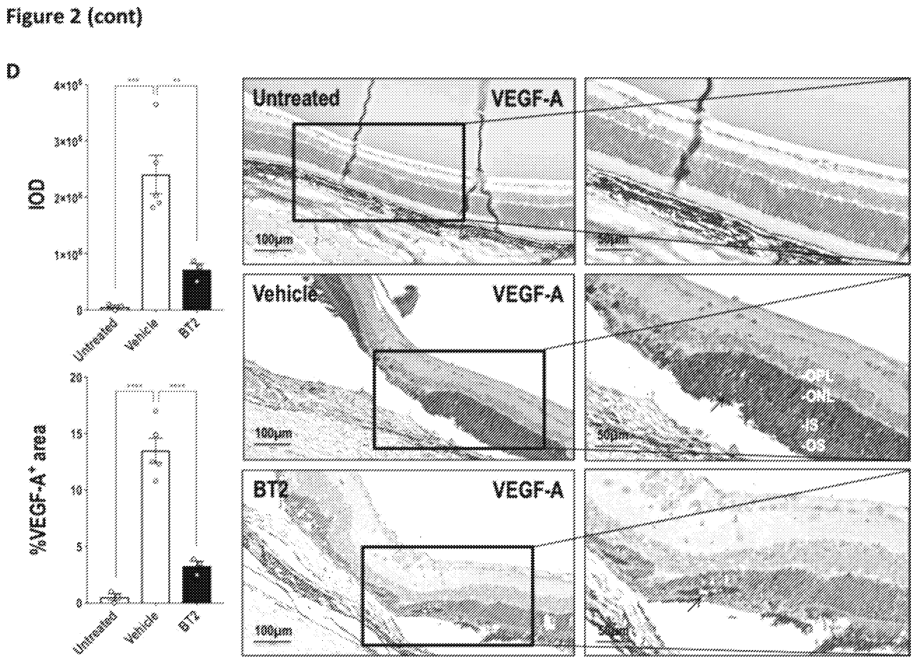

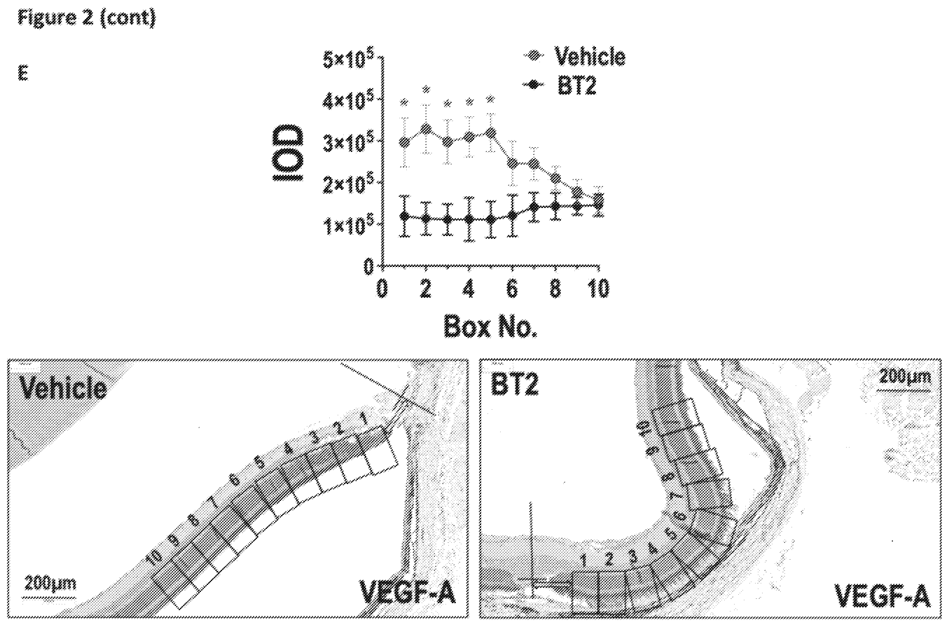

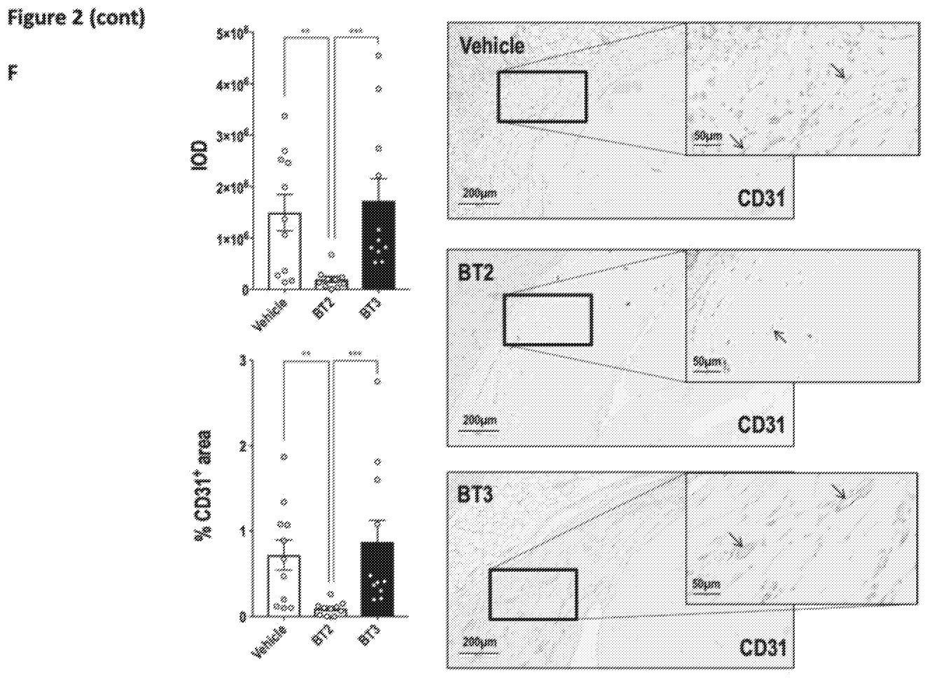

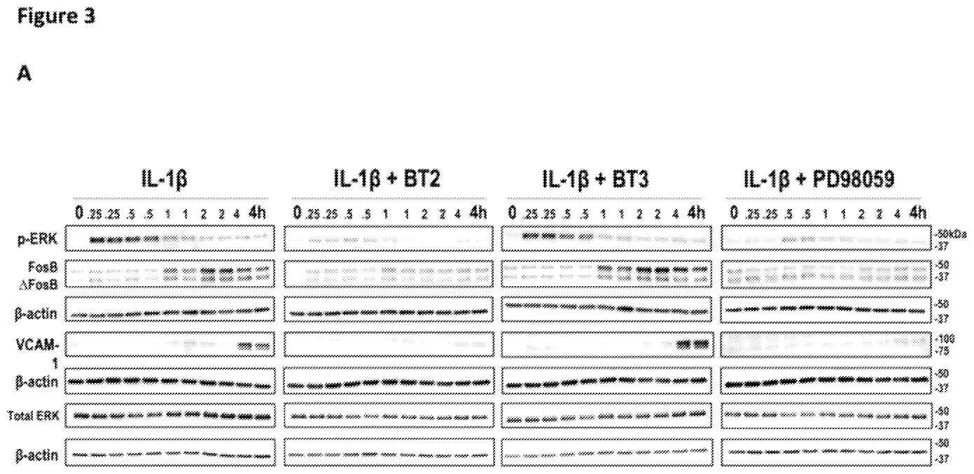

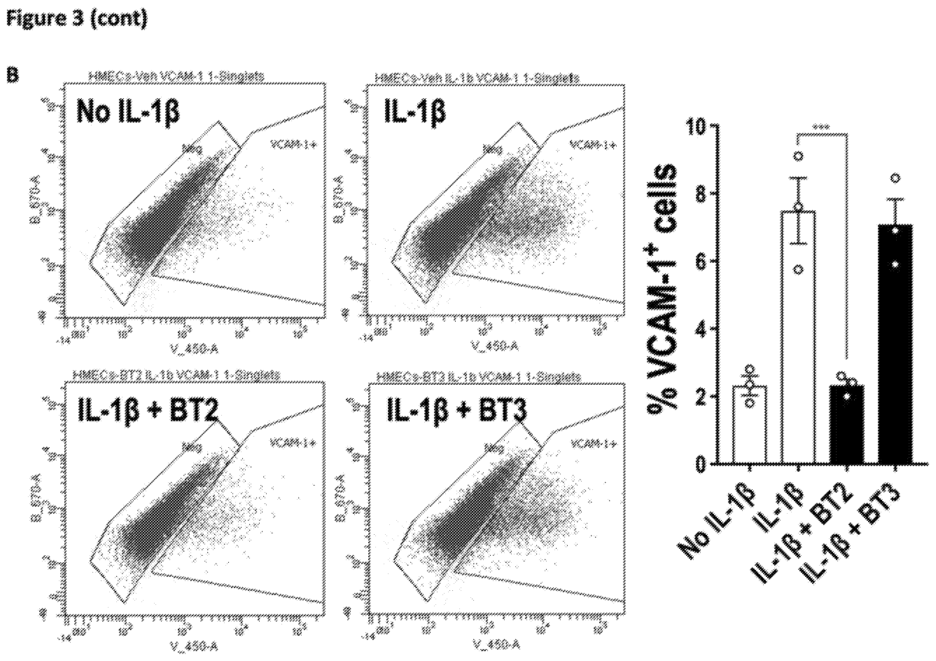

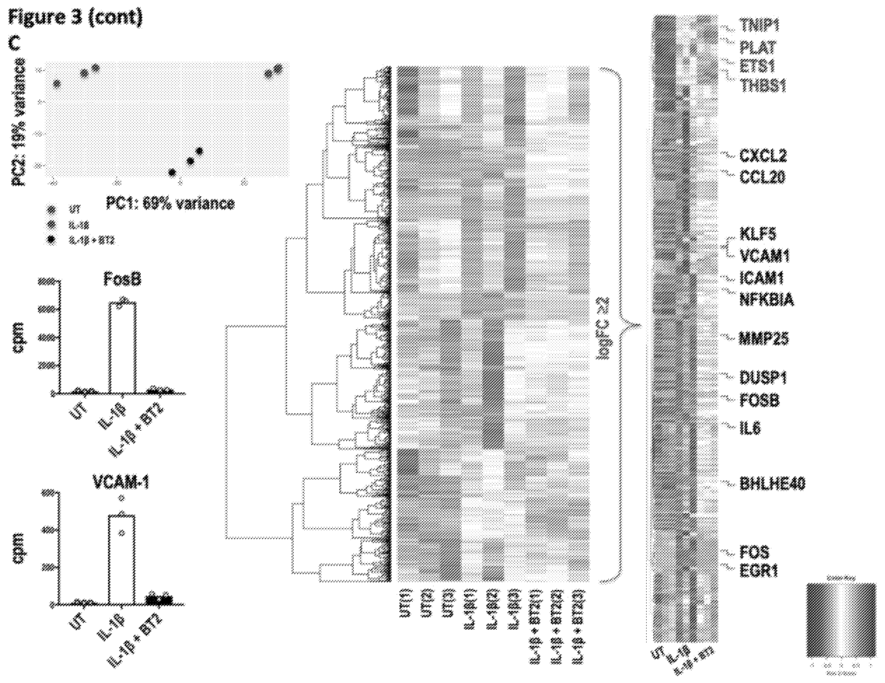

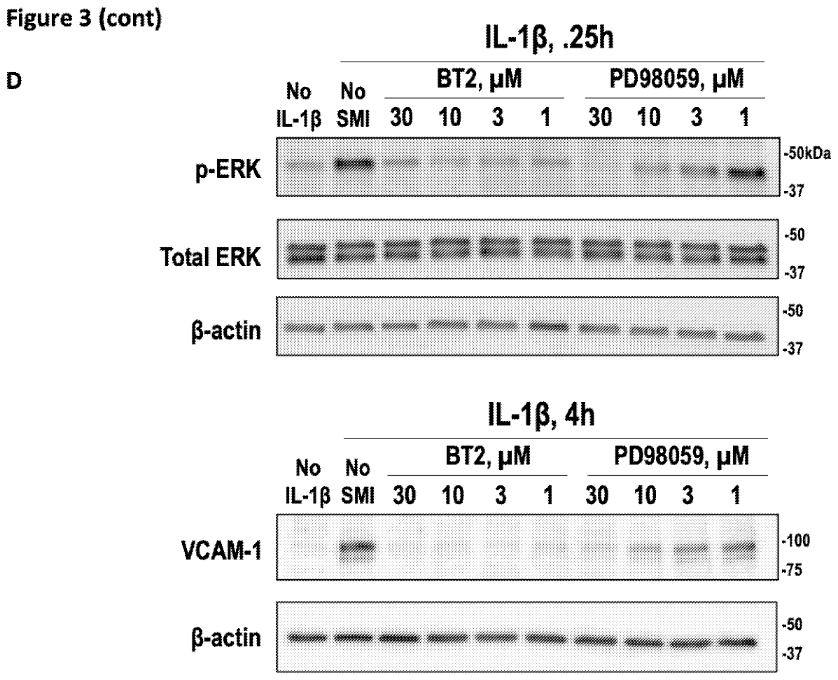

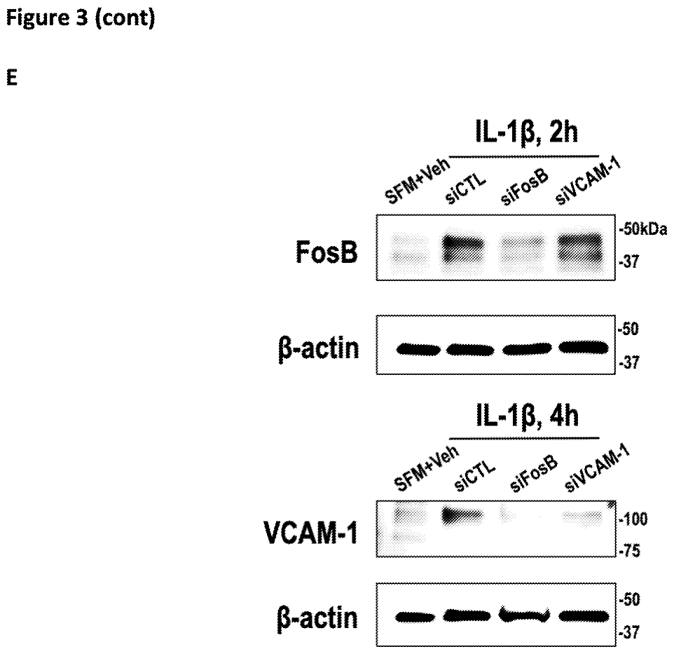

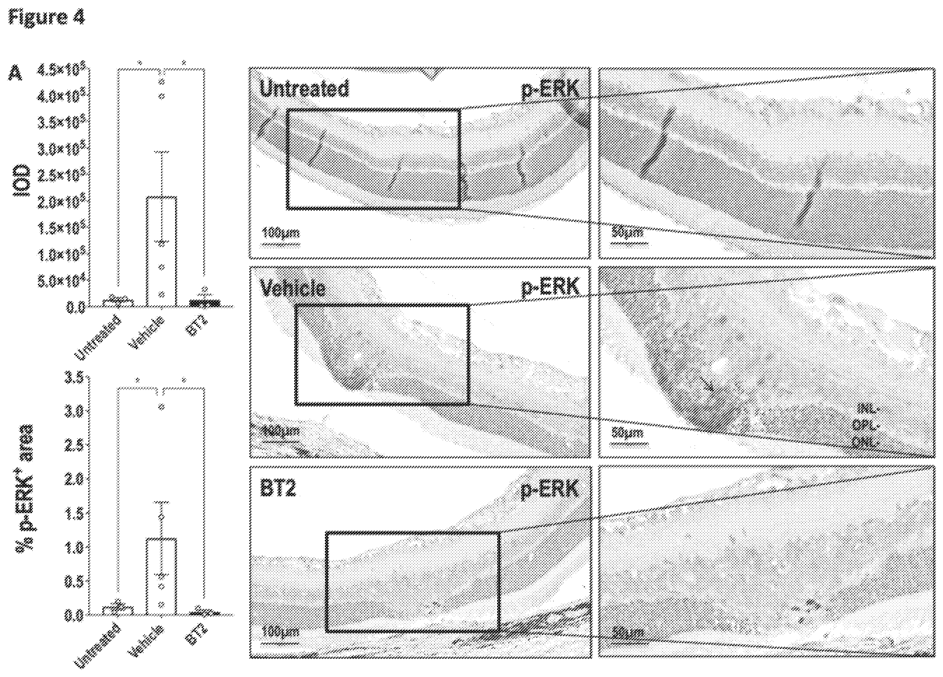

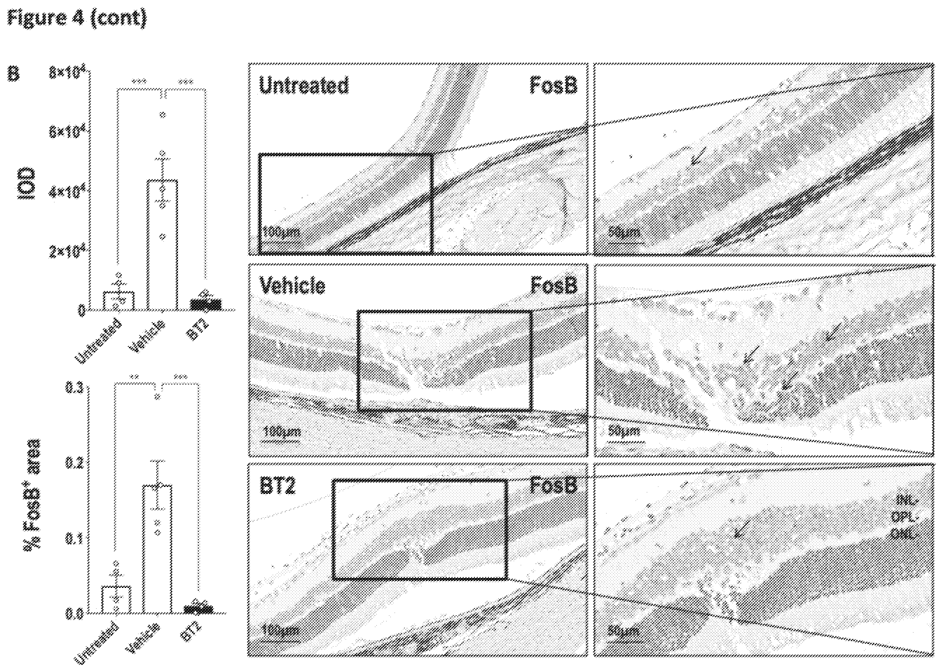

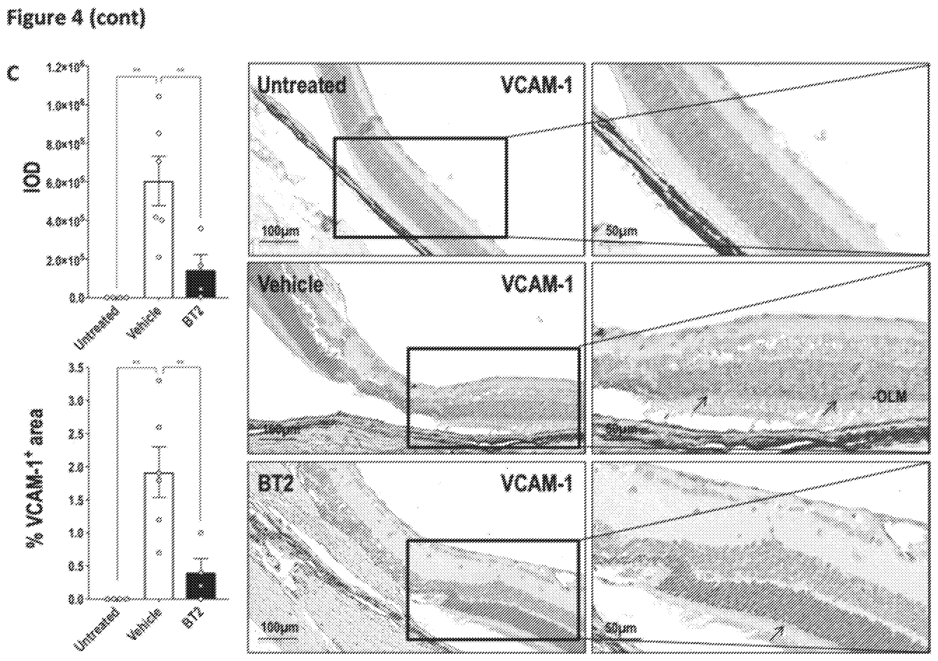

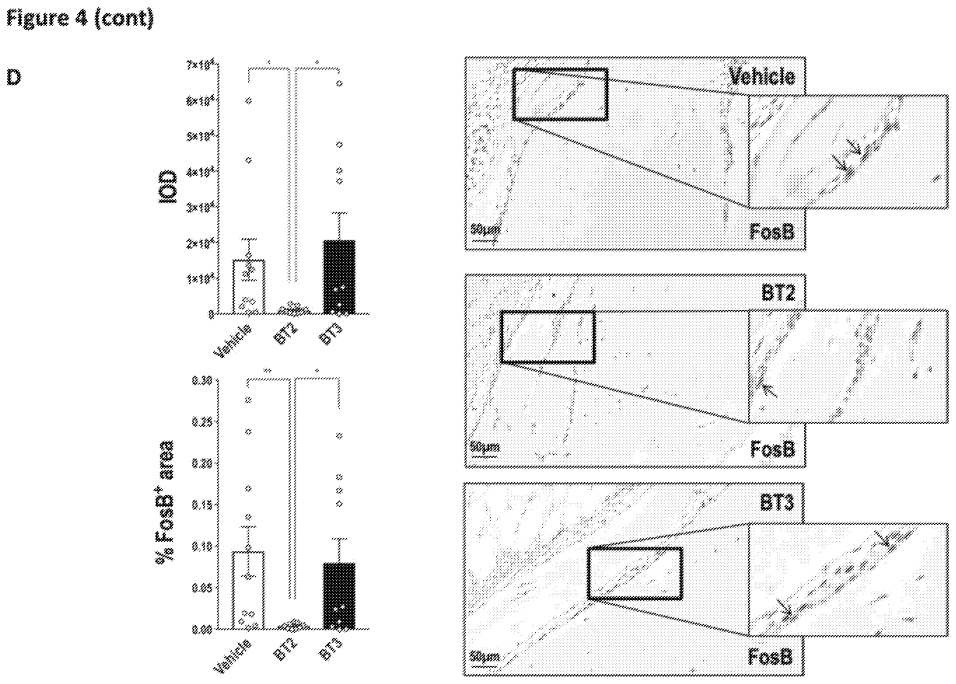

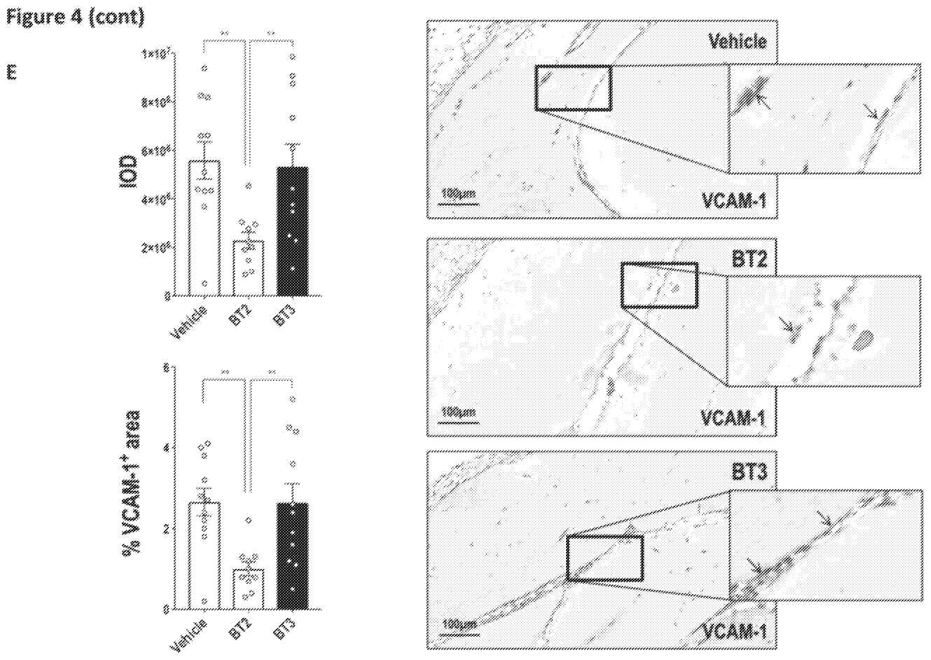

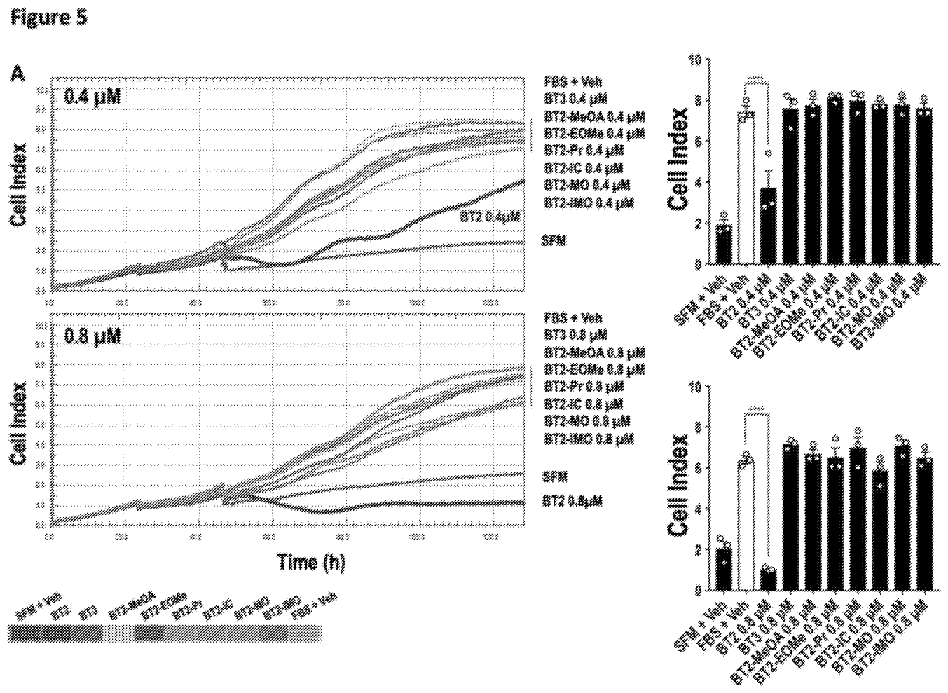

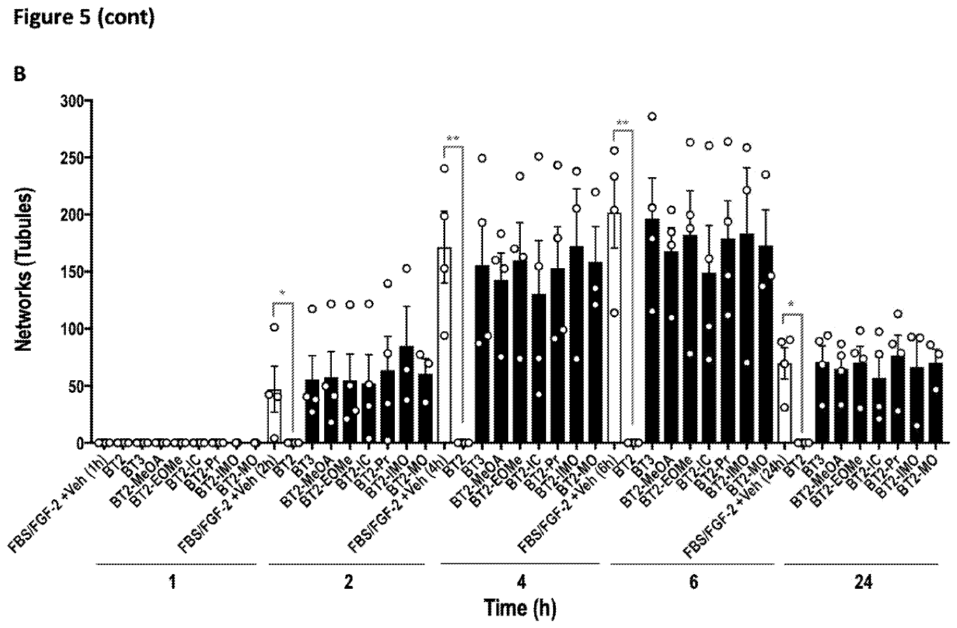

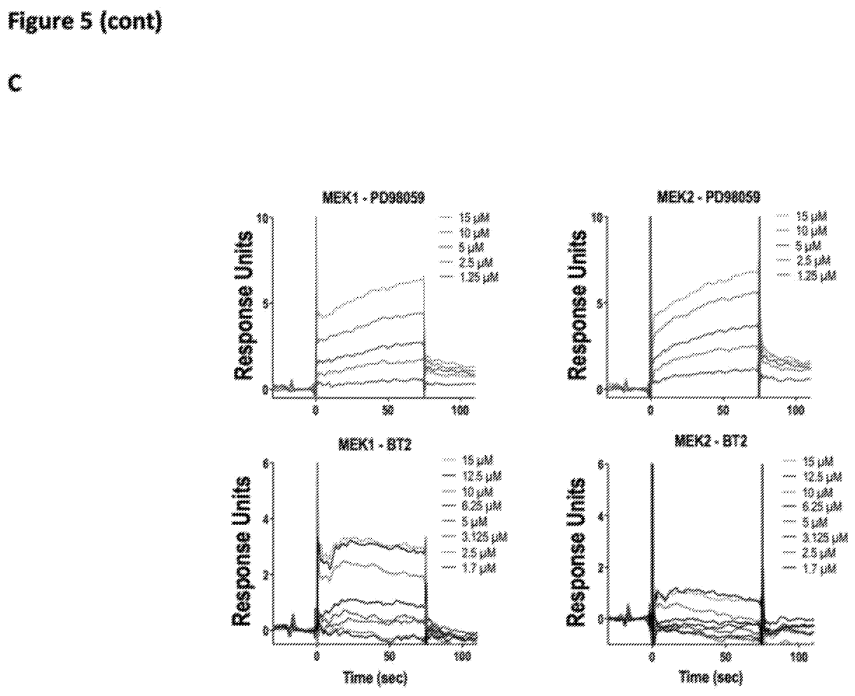

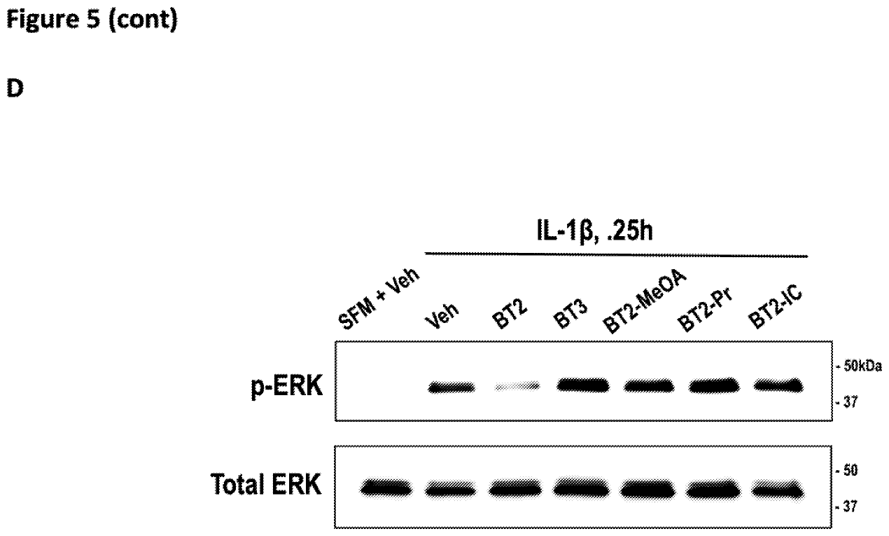

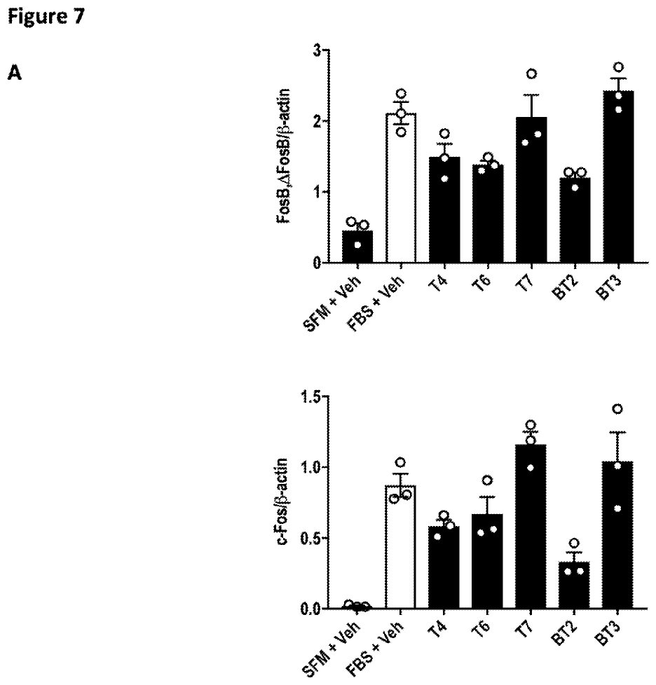

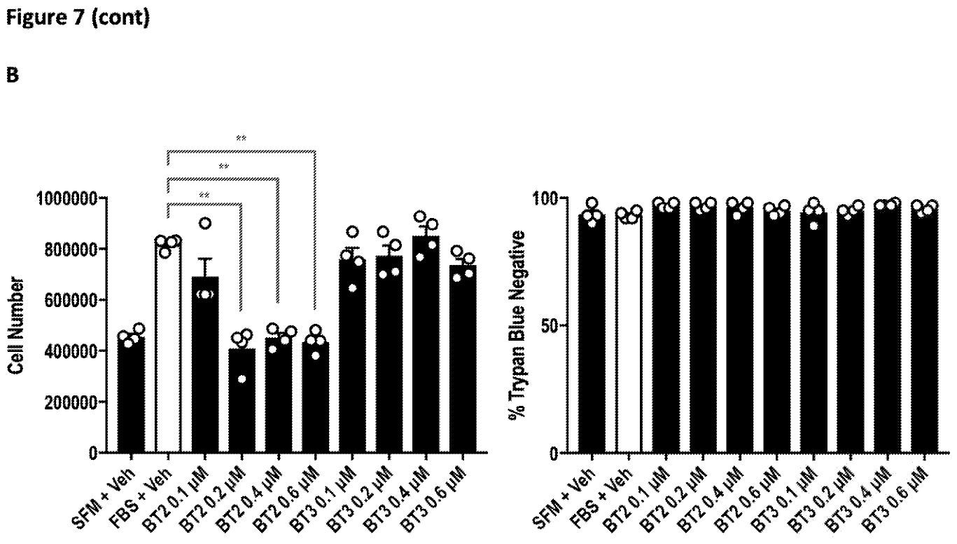

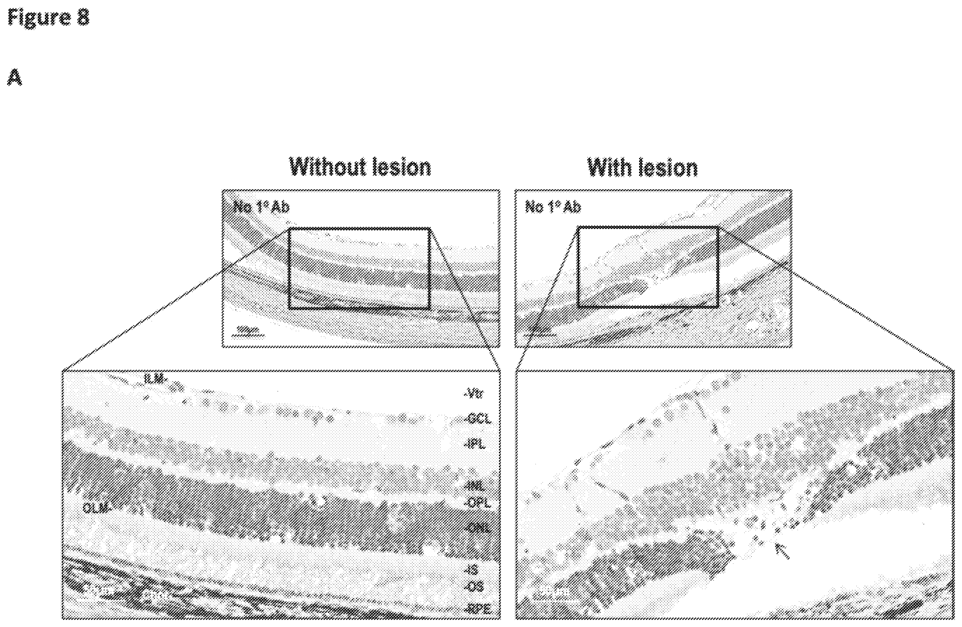

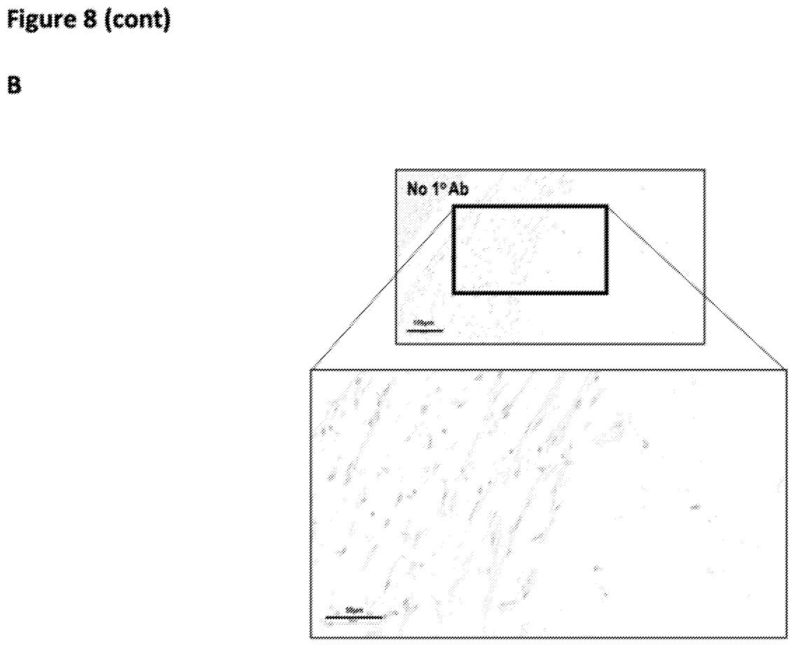

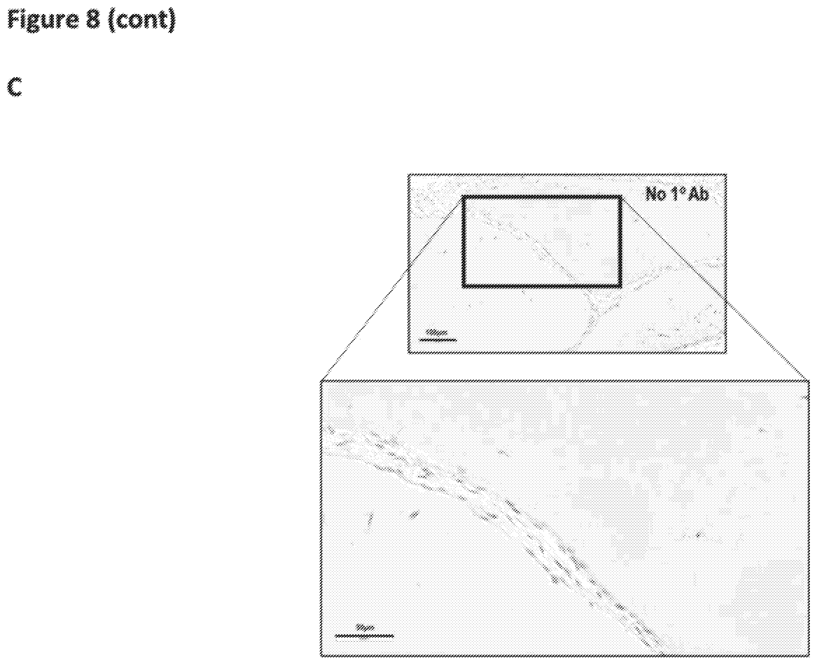

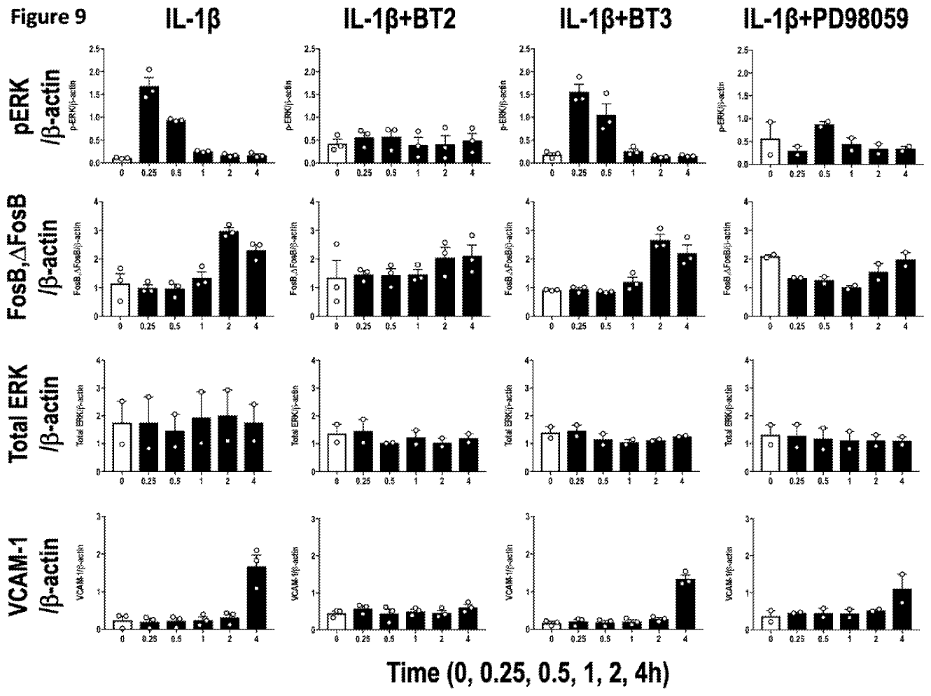

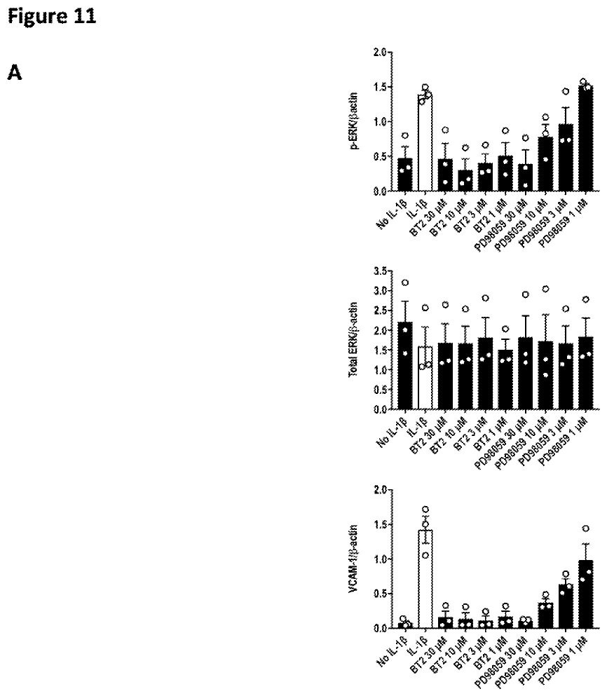

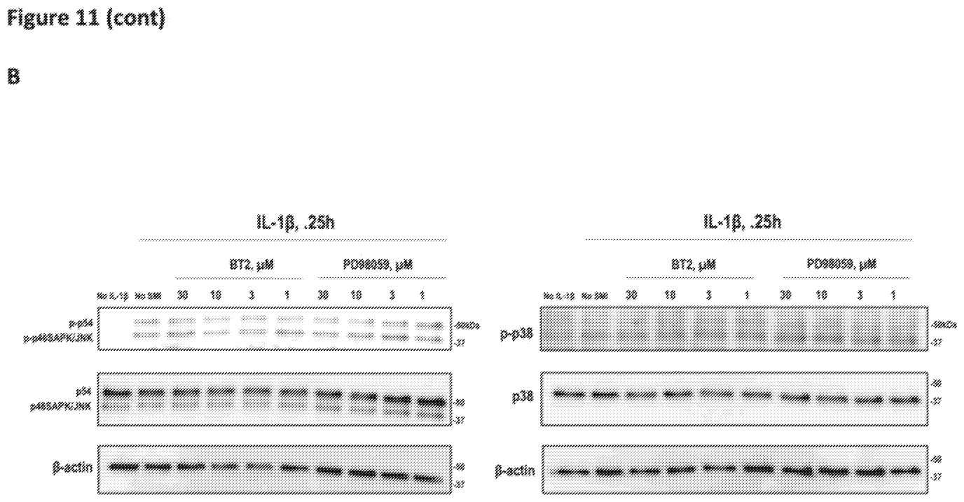

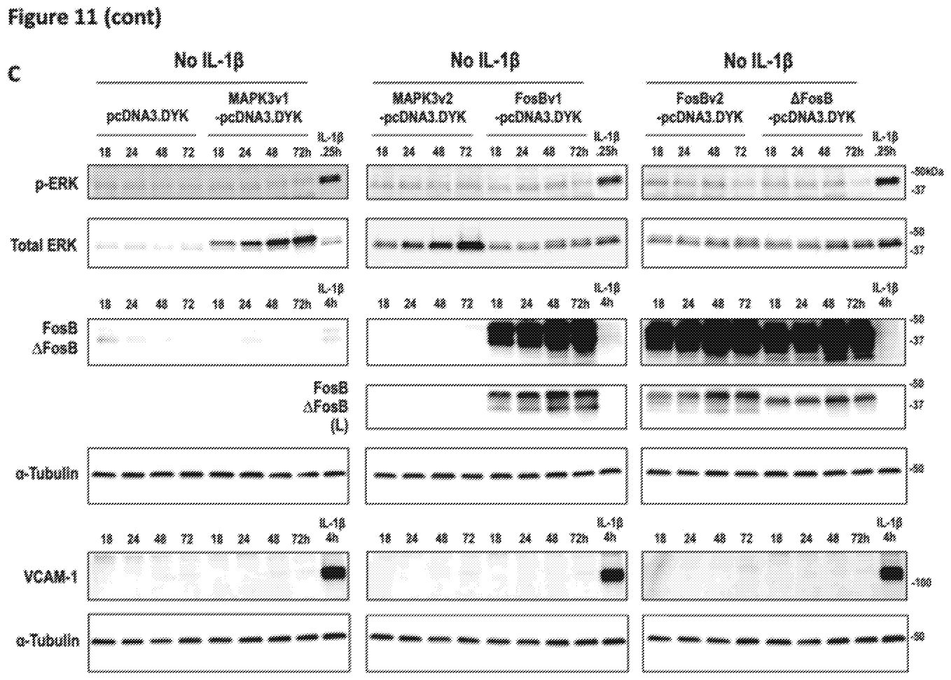

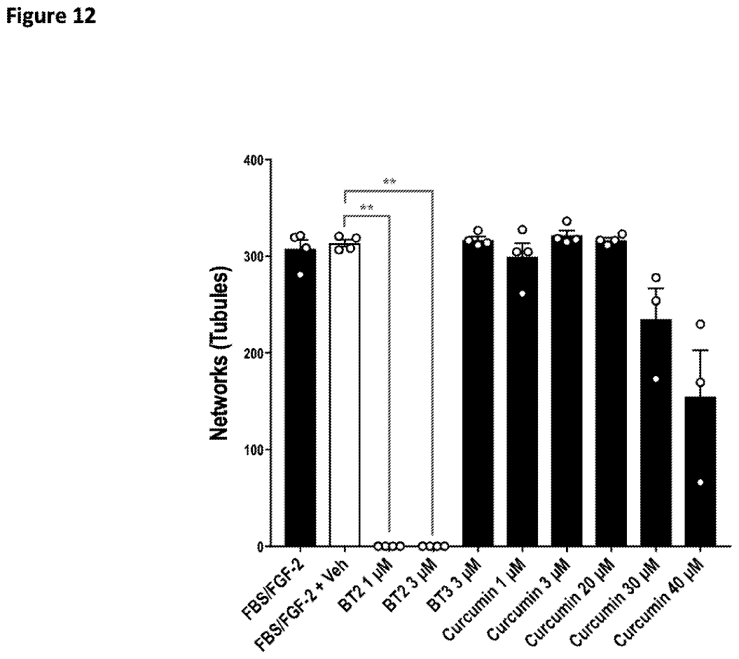

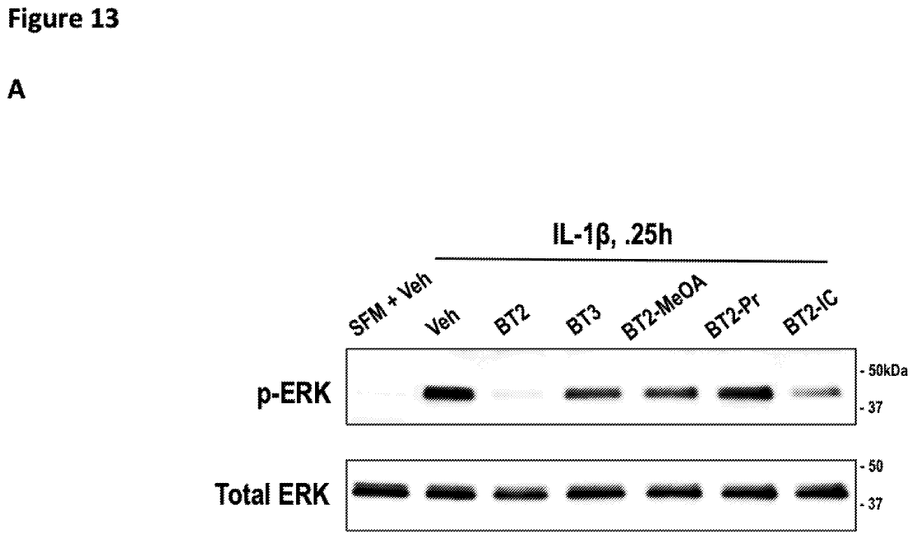

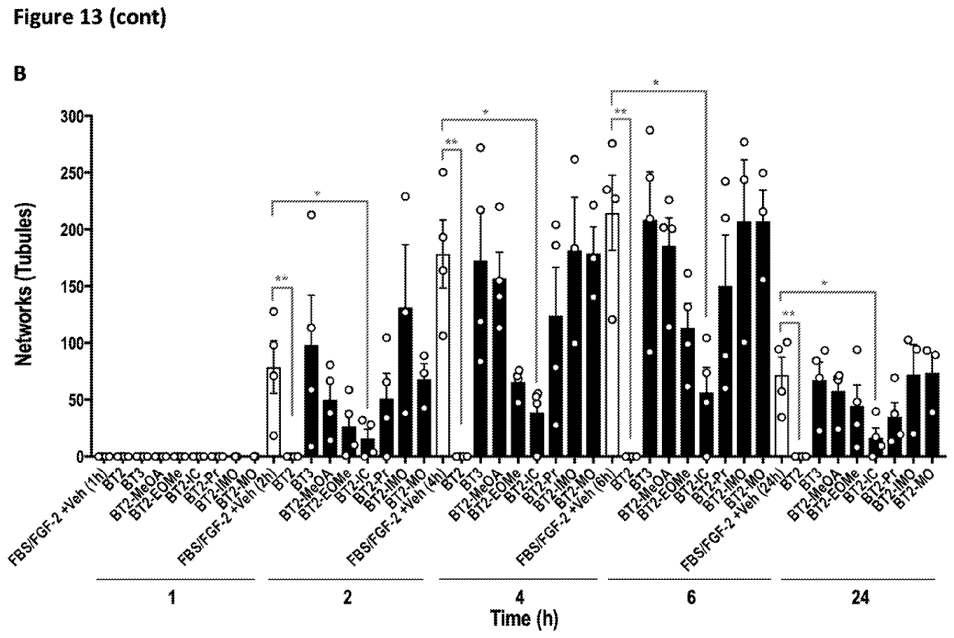

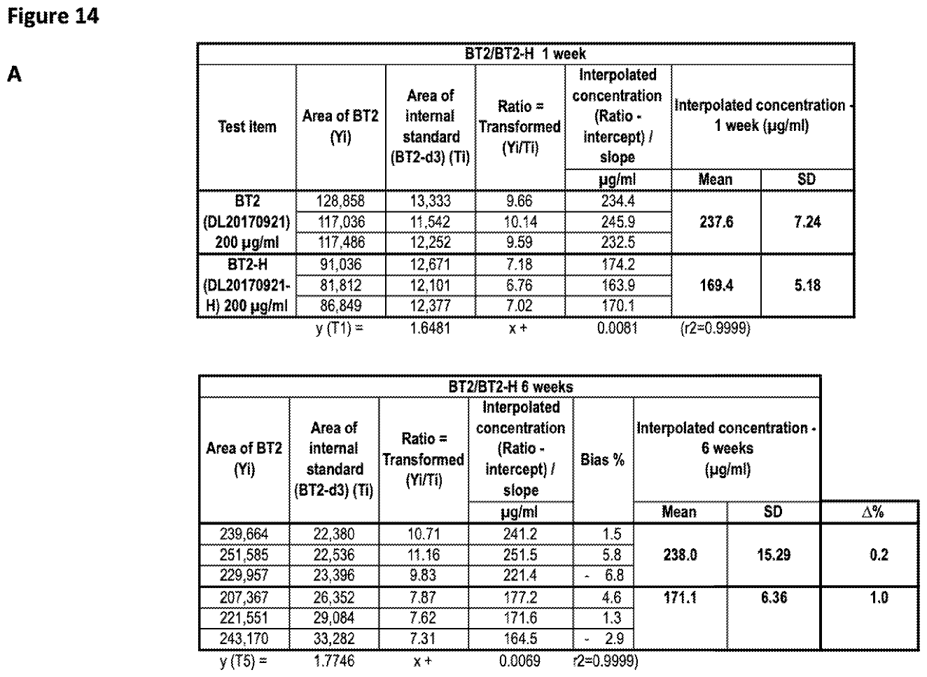

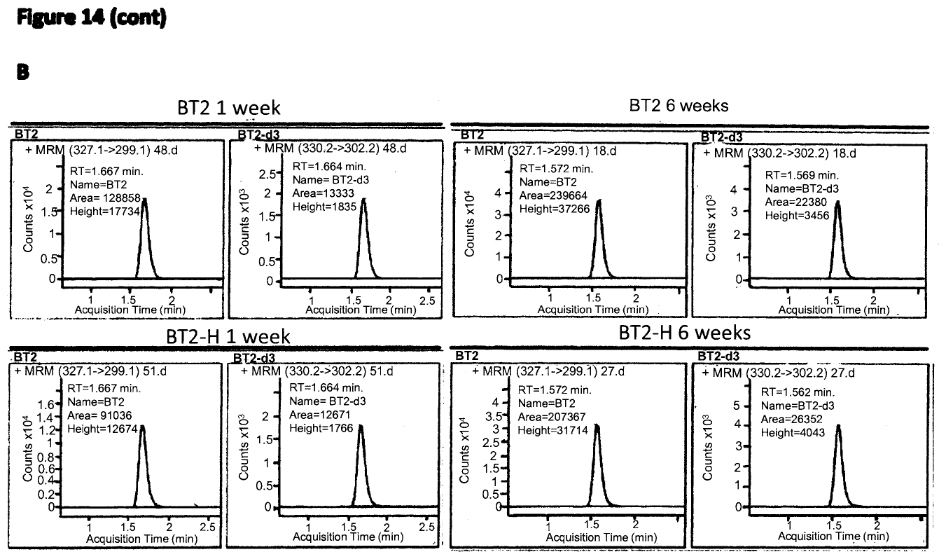

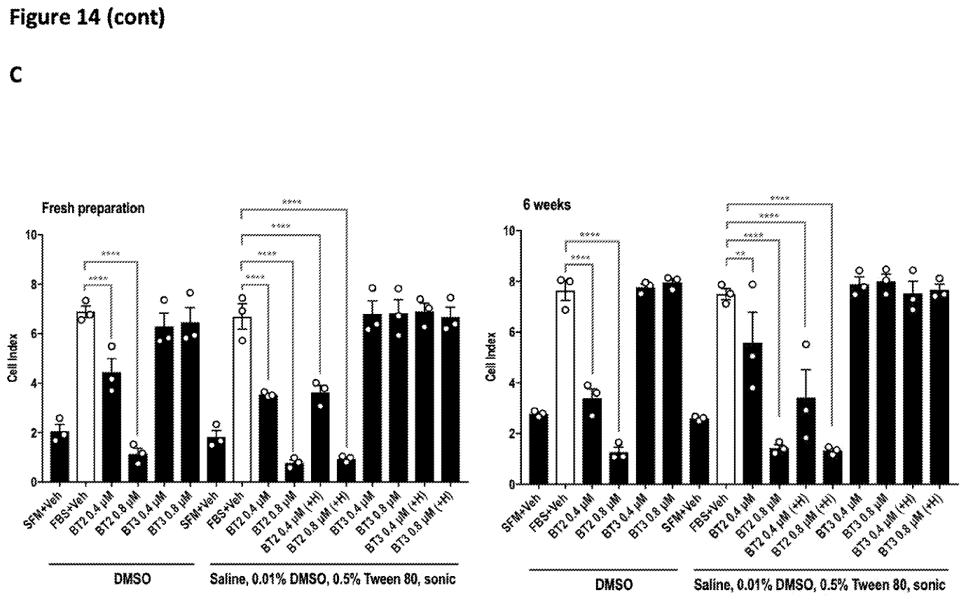

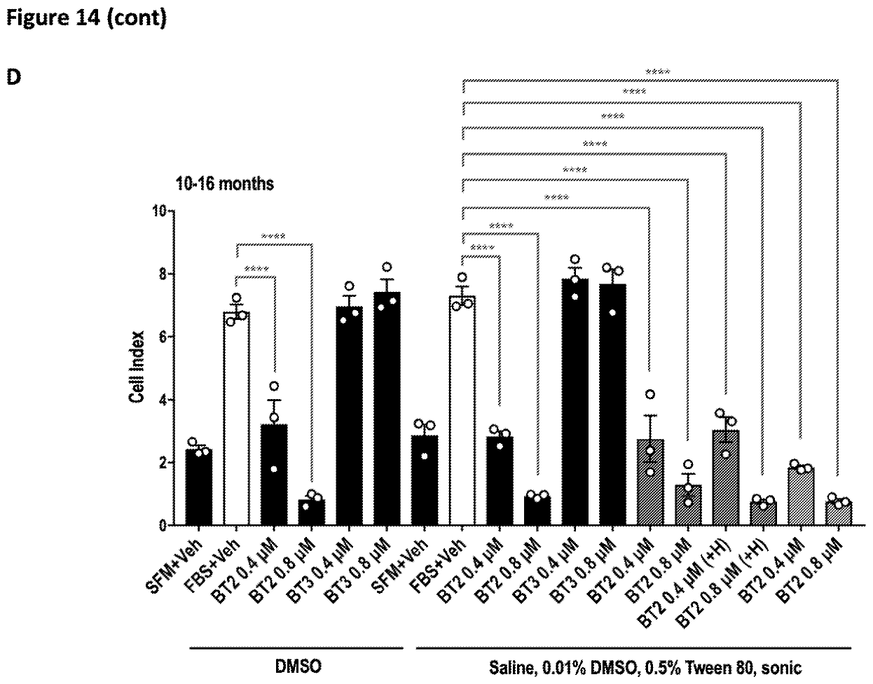

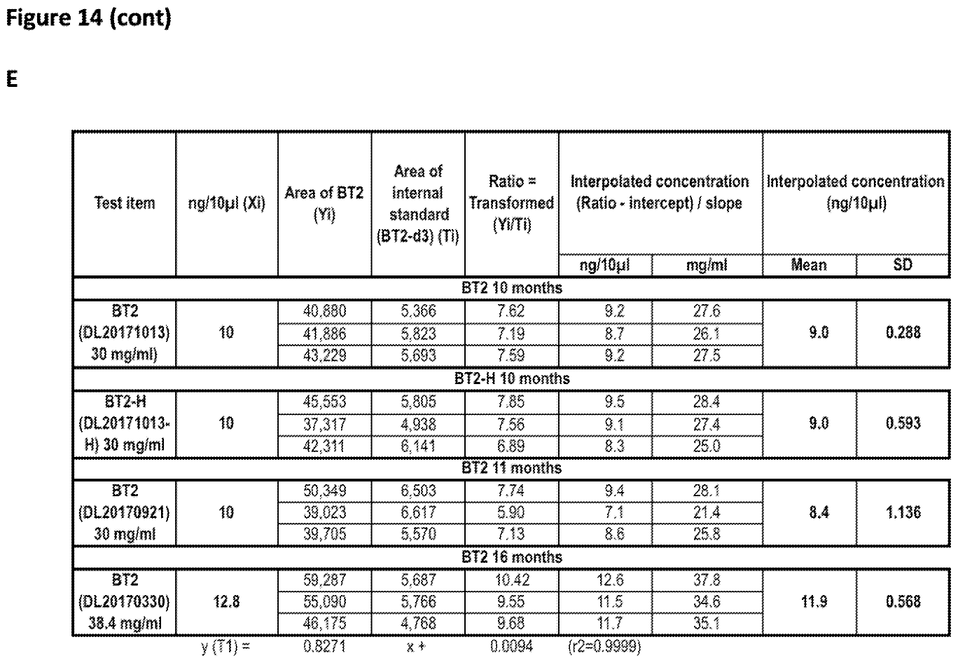

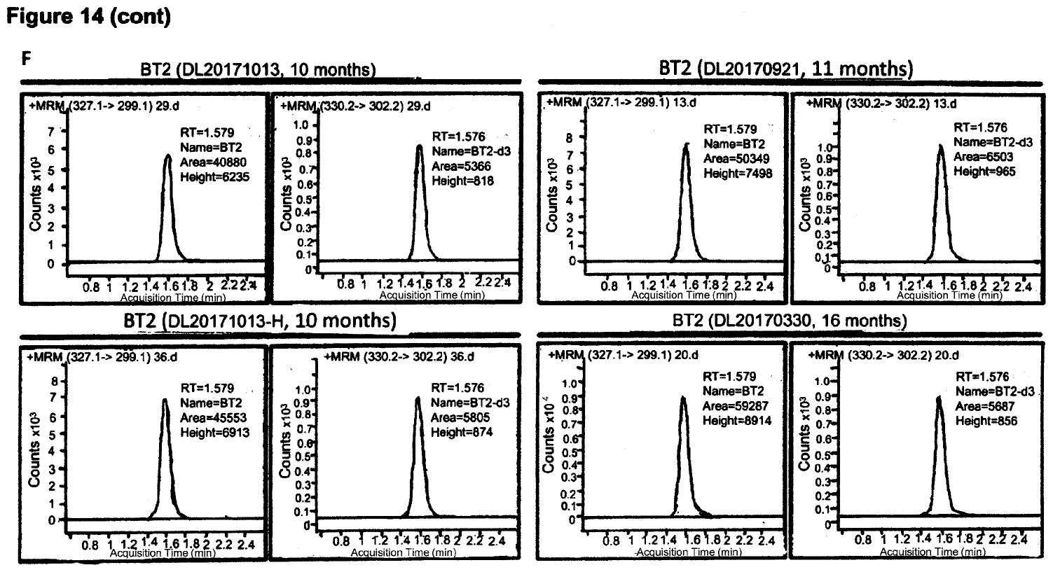

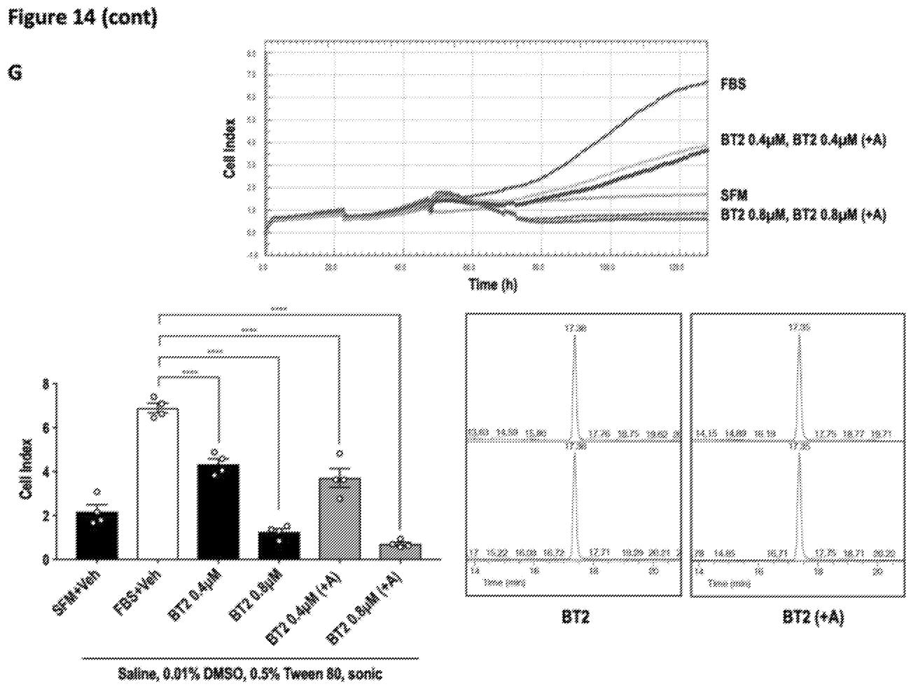

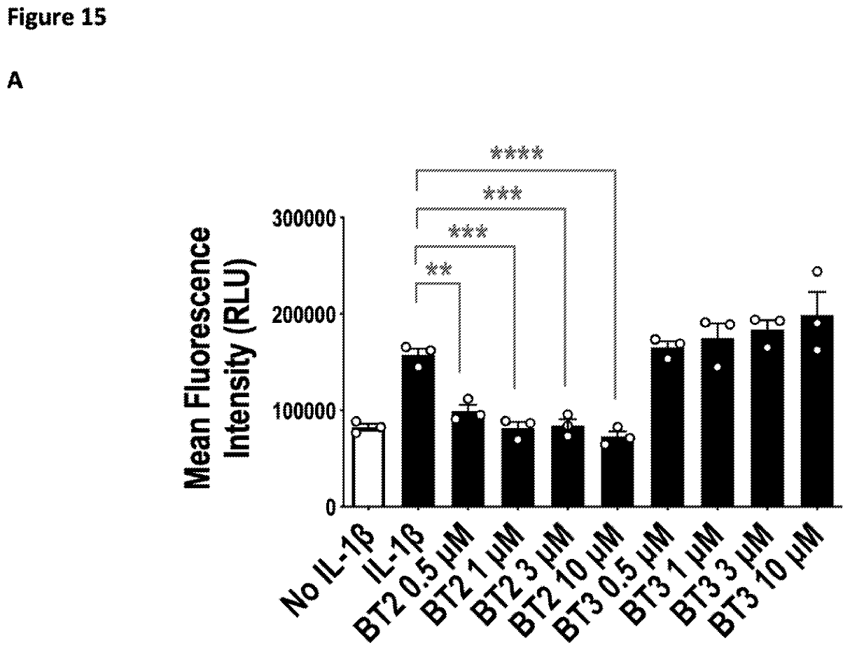

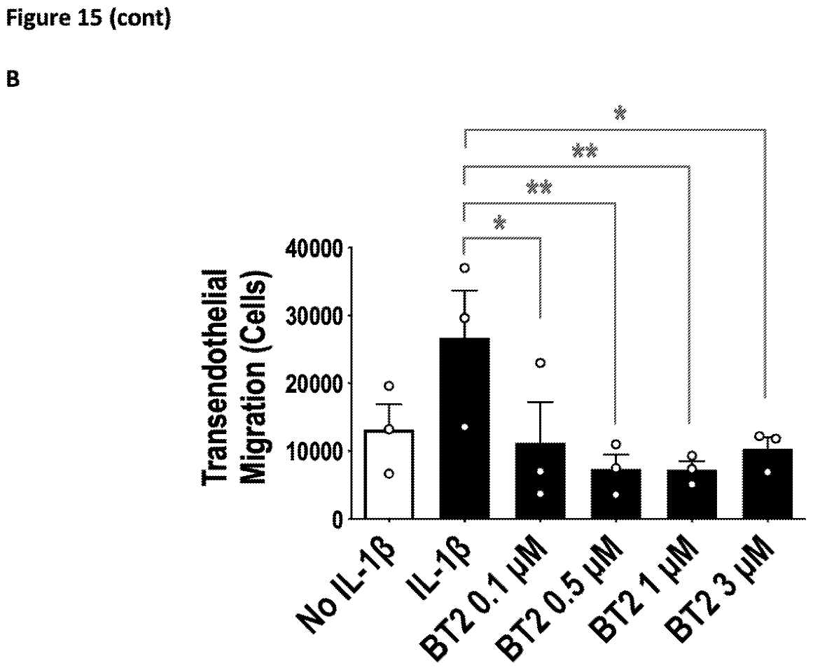

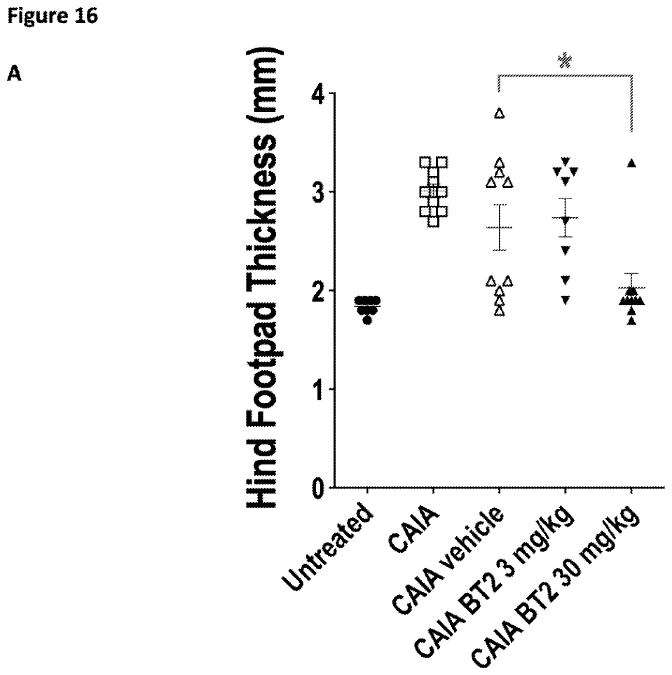

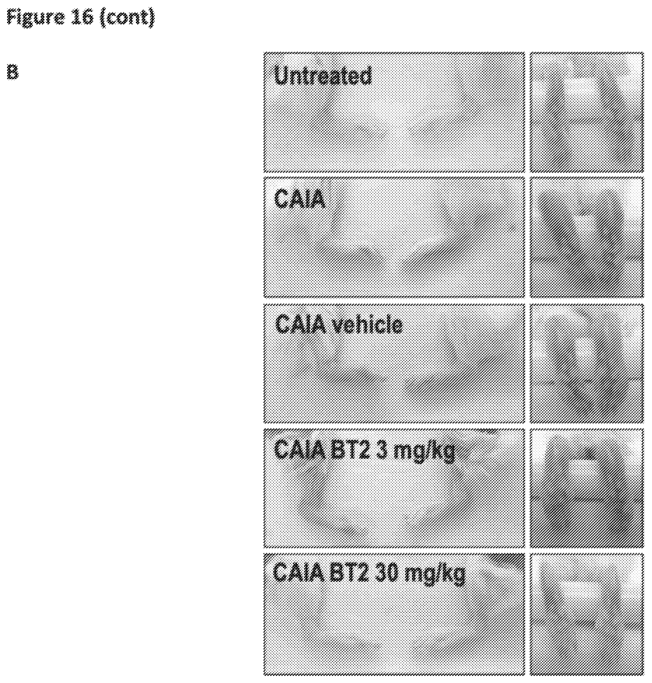

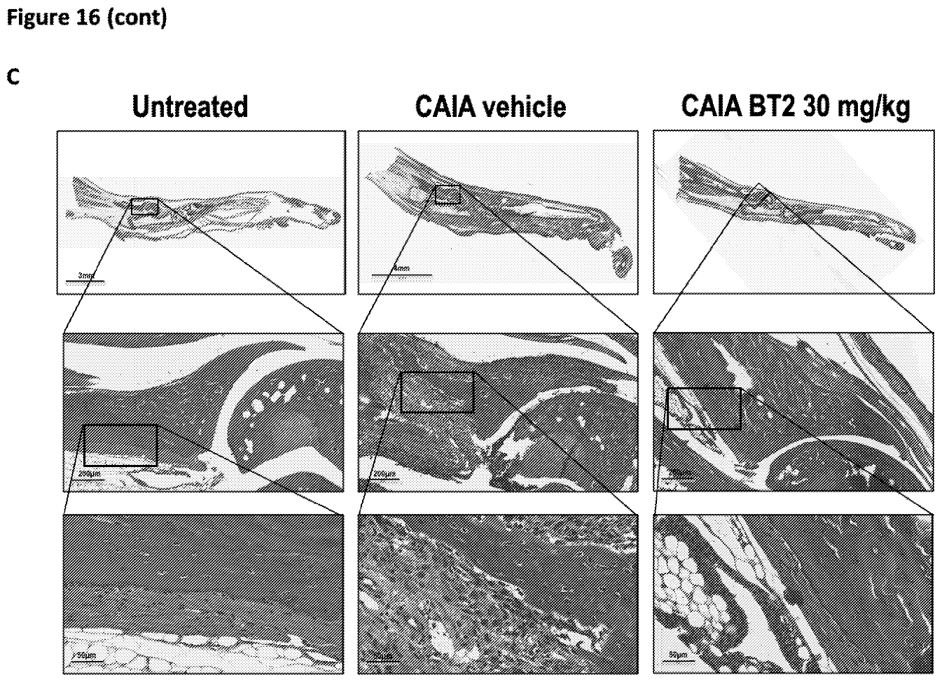

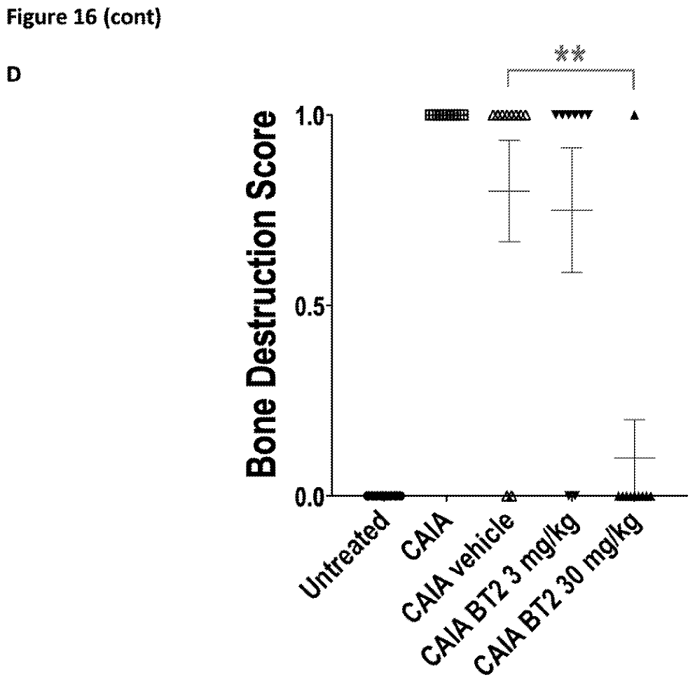

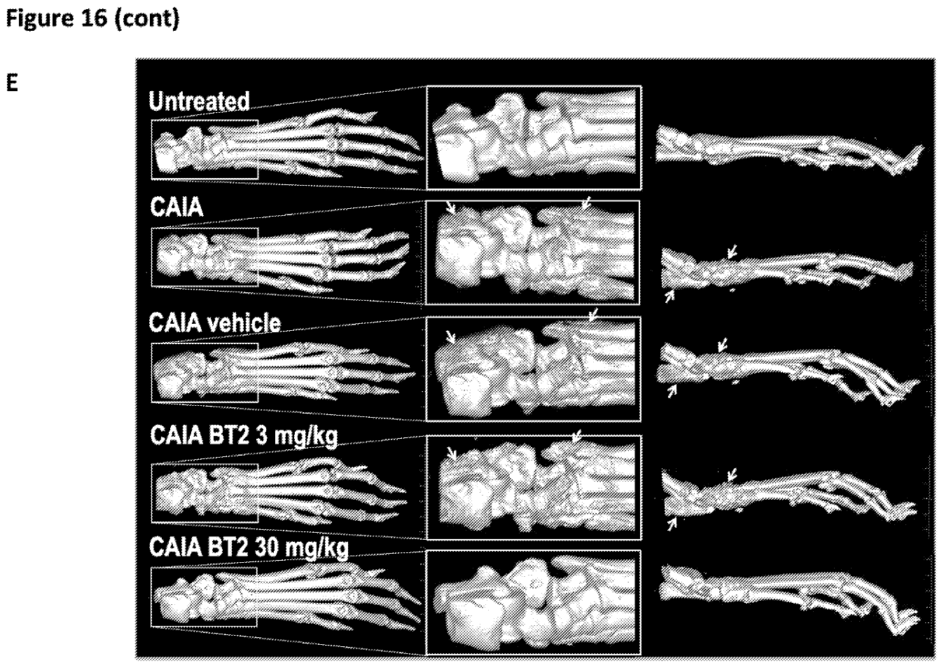

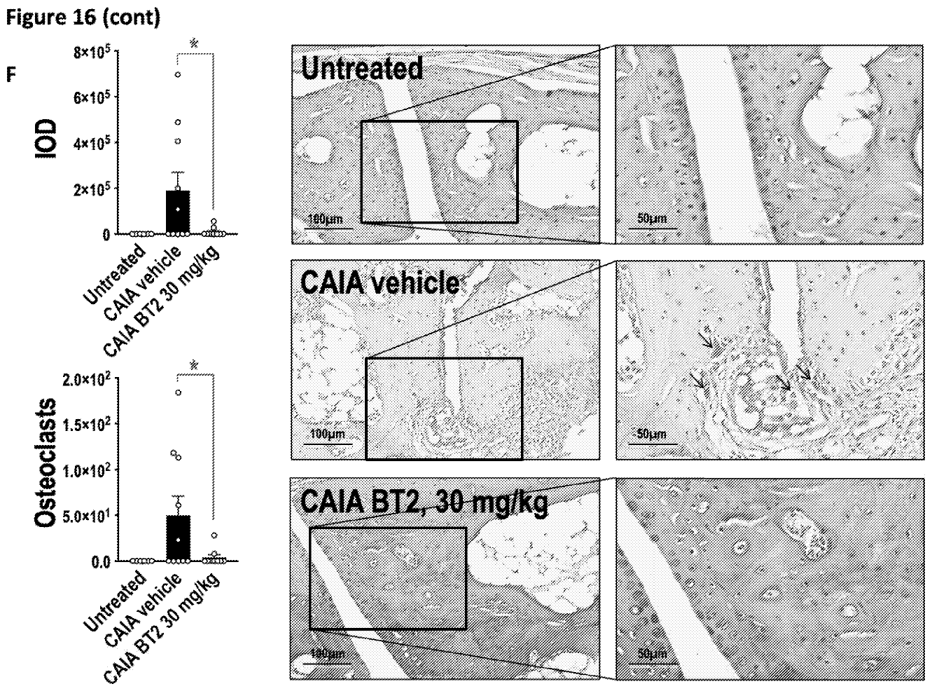

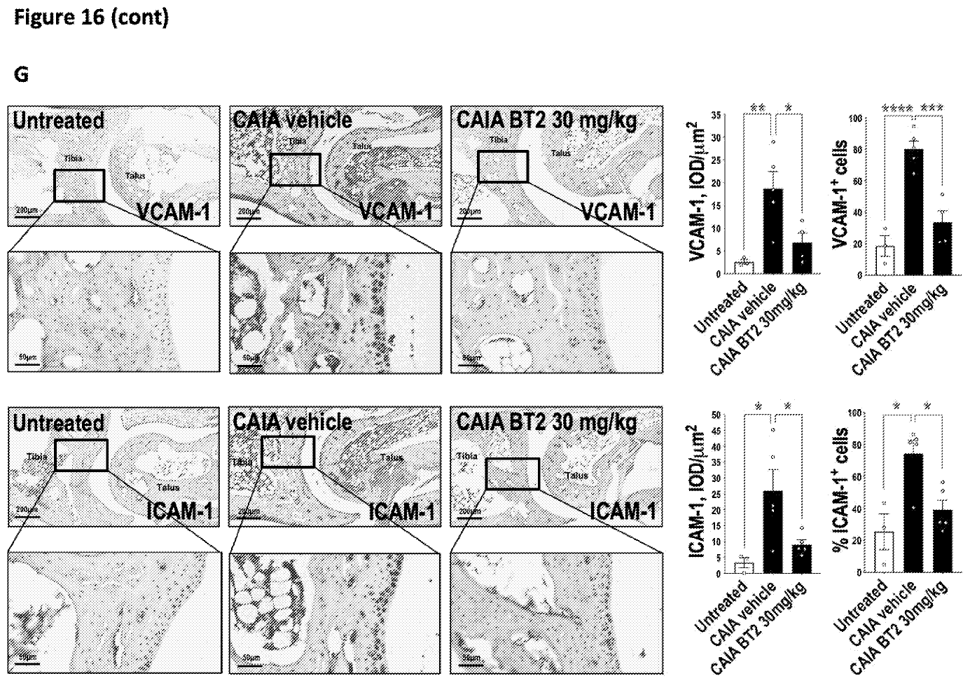

Identification of BT2, T4, T6. To identify novel small molecule inhibitors of AP-1, the ˜100,000 compound WEHI Lead Discovery Library was screened using a 293 cell-based assay in which Firefly luciferase was driven by multiple copies of the AP-1 response element. A substructure filter was applied during the course of screening to remove pan assay interference compounds (PAINS) (Baell, J. B., et al., J Med Chem 53, 2719-2740 (2010)) that typically captures the AP-1 inhibitor curcumin (Nelson, K. M., et al., J Med Chem 60, 1620-1637 (2017)). This yielded 24 available hits with IC 50 in the micromolar or submicromolar range determined using 11-point titration curves, including the dibenzoxazepinone BT2. This followed an earlier screen of a 960 compound DIVERSet library (ChemBridge) that yielded the benzophenone Cpd B/X/LK001 (FIG. 6A). T4, T6 and T7 are structural analogues of Cpd B/X/LK001, whereas BT3 is an analogue of BT2 (Table 1). BT2 was synthesized subsequent to the screen by reacting commercially available 2-amino-10-ethyldibenzo[b,f][1,4] oxazepin-11 (10H)-one (BT3) with diethyl pyrocarbonate (FIG. 6B, Scheme 1). Cpd B/X/LK001 was produced by reacting 2-methoxyethyl carbonisocyanatidate (2) (Krebs, A, et al., European Patent Office EP0230224B1 (1991)) with the commercially available (4-aminophenyl)(4-chlorophenyl)methanone (1) (FIG. 6B, Scheme 4). Treatment of Cpd B/X/LK001 with hydroxylamine hydrochloride gave T4 as a ˜1:1 mixture of E and Z isomers (FIG. 6B, Scheme 4). Flubendazole (T6) and (4-aminophenyl)(4-fluorophenyl)methanone (T7) were sourced commercially.
BT2, T4 and T6 inhibit serum-inducible endothelial FosB/ΔFosB and c-Fos expression, and block proliferation, migration and network formation in vitro. We determined the effect of BT2, T4 and T6 on the serum-inducible expression of two different AP-1 subunits in cultured human microvascular endothelial cells (HMEC-1). Endothelial cells provide a vital barrier between the flowing blood and tissue that become hyperpermeable when activated or stressed (van Hinsbergh, V. W., et al., Arterioscier Thromb Vasc Biol 7, 1018-1023 (1997)). BT2 blocked the inducible expression of FosB and ΔFosB (FIGS. 1A & 7A). T4 and T6 inhibited less potently, while BT3 and T7 demonstrated no inhibition (FIG. 1A). BT2 also blocked the inducible expression of c-Fos, a known mediator of angiogenesis (Marconcini, L., et al., Proc Natl Acad Sci USA 96, 9671-9676 (1999)) (FIGS. 1A & 7A).
We next investigated the effects of these compounds on endothelial cell growth using the xCELLigence system that monitors cell proliferation in real time. We found that BT2, T4 and T6 each inhibited serum-inducible proliferation at concentrations in a dose-dependent manner (FIG. 1B). In contrast, BT3 or T7 had no inhibitory effect. To confirm that growth inhibition was not merely due to cell death, we tested one of these compounds (BT2) in a whole cell proliferation assay in which the Countess system is coupled with Trypan Blue exclusion. BT2 inhibited serum-inducible proliferation without affecting Trypan Blue incorporation (FIG. 7B). In the dual chamber Transwell system, BT2, T4 and T6 inhibited migration of bovine aortic endothelial cells (BAEC) toward VEGF-A₁₆₅ in serum-containing medium (FIG. 1C). BAEC were used for this purpose since HMEC-1 cells lack VEGFR-2 (Flk/KDR) and only weakly migrate toward VEGF (Shao, R., et al., Biochem Biophys Res Commun 321, 788-794 (2004)). BAEC, on the other hand, express VEGFR-2 (Lamy, S., et al., Cancer Res 62, 381-385 (2002)) and migrate to VEGF-A (Hussain, S., et al., BMC Cell Biol 9, 7 (2008)). In contrast, neither BT3, nor PD98059 (allosteric MEK inhibitor), imatinib (tyrosine kinase inhibitor) or tofacitinib (Janus kinase inhibitor) had an inhibitory effect at the same concentration (FIG. 1C).
Endothelial cell repair after mechanical injury in vitro evokes a proliferative and migratory response. BT2, T4 and T6 blocked this reparative response within 48 h, whereas BT3 or T7 had no such effect (FIG. 1D). We also evaluated the effect of these compounds in an endothelial network formation assay (also known as tubule formation) atop reconstituted basement membrane matrix typically used to characterize angiogenic factors and processes (Arnaoutova, I., et al., Angiogenesis 12, 267-274 (2009)). Endothelial cells in this assay form capillary-like networks maximally within a few hours and regress thereafter. BT2, T4 and T6 inhibited network formation at 2, 4, 6 and 24 h (FIG. 1E).
BT2 prevents retinal vascular permeability and angiogenesis. Since retinal vascular permeability is a key pathologic feature in nAMD and DME/DR (Campochiaro, P. A., et al., J Mol Med (Berl) 91, 311-321 (2013)), we sought to determine the effect of BT2, T4 and T6, on fluorescein leakage induced in eyes of Brown Norway pigmented rats after multiple laser burns of Bruch's membrane around the optic nerve (Grossniklaus, H. E., et al., Prog Retin Eye Res 29, 500-519 (2010)). BT2 (192n) reduced retinal permeability by ˜50%, an effect similar to aflibercept/Eylea (200 μg administered 6 times (Days 0, 3, 7, 10, 14, 17) by intravitreal (IVT) injection over 21 days as compared with 2 injections (Days 0, 7) of BT2) or triamcinolone acetonide (Kenacort® 200 μg IVT, Day 0) (FIG. 2A). In contrast, T4 and T6, delivered as per BT2, had no inhibitory effect (FIG. 2A). Aflibercept is first-line therapy for nAMD and DME in the US, Europe and Asia-Pacific (Parikh, R., et al., Ophthalmol Retina 3, 16-26 (2019), while Kenacort is a corticosteroid commonly used to treat DME (Karacorlu, M., Eye (Lond) 19, 382-386 (2005)). BT2 also reduced vascular permeability induced by rhVEGF-A₁₆₅ in pigmented rabbits causing fluorescein leakage. Single IVT delivery of BT2 (600n) inhibited retinal leakage after 2 days by ˜50% (FIG. 2B). Immunohistochemical staining of lasered rat eyes 21 days after injury revealed that BT2 inhibited inducible CD31 staining in the IPL and INL (FIGS. 2C & 8A), where CD31 is expressed after laser injury (Ju, X., et al., Clin Exp Pharmacol Physiol 46, 75-85 (2019)). BT2 also inhibited the inducible expression of VEGF-A₁₆₅ (FIG. 2D), consistent with findings of VEGF expression mainly in the outer retina (Wang, X., et al., Int J Mol Sci 8, 61-69 (2007); Foureaux, G., et al., Braz J Med Biol Res 48, 1109-1114 (2015)). VEGF-A₁₆₅ stained in a gradient relative to the wound which was inhibited by BT2 (FIG. 2E). The murine Matrigel plug assay confirmed the anti-angiogenic properties of BT2. Matrigel containing VEGF-A₁₆₅, heparin and compound was implanted subcutaneously into C57BL/6 mice and CD31 staining in plugs after 7 days was quantified. BT2 suppressed new blood vessel formation, whereas BT3 had no effect (FIGS. 2F & 8B).
BT2 inhibits ERK phosphorylation, FosB/ΔFosB and VCAM-1 expression. Endothelial cells exposed to IL-1β undergo rapid ERK phosphorylation. IL-1β causes endothelial cell permeability (Puhlmann, M., et al., J Transl Med 3, 37 (2005)) and retinal leukostasis (Vinores, S. A., et al., J Neuroimmunol 182, 73-79 (2007)). Diabetics with macular edema have significantly higher concentrations of IL-1β among other cytokines and VEGF in the aqueous humor (Dong, N., et al., PLoS ONE 10, e0125329 (2015)). We used IL-1β as a model agonist with HMEC-1 in Western blotting experiments. BT2 inhibited IL-β-inducible ERK phosphorylation, FosB/ΔFosB and VCAM-1 expression (FIGS. 3A & 9). BT2 inhibition of VCAM-1 was further demonstrated by flow cytometry (FIGS. 3B & 10).
RNA-sequencing affirmed BT2's ability to suppress IL-1β-inducible FosB and VCAM-1 expression (FIG. 3C). From a pool of 33379 gene IDs, there were 325 genes induced by IL-1β 2-fold or more (logFC2) (Table 3C), 89 (27.5%) of which were inhibited by BT2 (logFC≥2) (Table 3B). Principal component analysis (PCA) (FIG. 3C, upper left) showed close association between biological replicates. BT2 also inhibited a range of other regulatory genes involved in cell proliferation, migration, angiogenesis and inflammation including ICAM-1, CXCL2, KLF5, Egr-1 and Fos (FIG. 3C).
Dose escalation and Western blotting experiments revealed that BT2 inhibited VCAM-1 and ERK phosphorylation more potently than PD98059 (FIGS. 3D & 11A). In contrast, BT2, like PD98059, had no effect on IL-1β-inducible p-SAPK/JNK or p-p38 (FIG. 11B). To explore the hitherto unrecognized dependence of VCAM-1 expression upon FosB/ΔFosB we performed siRNA knockdown experiments. FosB siRNA inhibited both FosB/ΔFosB and VCAM-1 whereas VCAM-1 siRNA inhibited VCAM-1 but not FosB/ΔFosB (FIG. 3E). Overexpression of ERK1 did not increase levels of phosphorylated ERK, compared with IL-1β stimulation, nor did it increase levels of FosB, ΔFosB or VCAM-1 (FIG. 11C). Similarly, overexpression of FosB or ΔFosB did not increase VCAM-1 expression compared with IL-1β stimulation (FIG. 11C). These findings indicate that, in this agonist-free system where ERK is not phosphorylated, FosB and VCAM-1 are not directly activated by ERK1 overexpression, nor is VCAM-1 directly activated by FosB or ΔFosB overexpression. These data complement our demonstration (under conditions of agonist stimulation) that BT2, which prevents ERK phosphorylation, abrogates the induction of FosB/ΔFosB and VCAM-1 in vitro (FIGS. 3A-D) and in vivo (FIGS. 4A-E). Indeed, BT2 physically interacts with MEK1 (FIG. 5C), which phosphorylates ERK (Qi, M., et al., Journal of Cell Science 118, 3569-3572 (2005)). These findings, taken together with our demonstration that IL-1β induction of VCAM-1 is blocked with FosB siRNA (FIG. 3E) suggest that while FosB is necessary for cytokine-inducible VCAM-1 expression, FosB overexpression alone is not sufficient to induce VCAM-1 without cytokine stimulation. FosB likely relies upon co-factors (or post-translational modifications) under conditions of cytokine stimulation.
Immunohistochemical staining of rat retinas revealed that BT2 suppressed inducible pERK staining in the INL, OPL and ONL (FIG. 4A) consistent with pERK expression in this proximity (Takeda, M., et al., Invest Ophthalmol Vis Sci 43, 907-911 (2002); Caicedo, A., et al., Exp Eye Res 81, 38-47 (2005)). BT2 also inhibited retinal FosB immunostaining (FIG. 4B). Moreover, BT2 reduced inducible VCAM-1 expression in the OLM (FIG. 4C) where others have found that VCAM-1 is expressed (Makhoul, M., et al., Exp Eye Res 101, 27-35 (2012)). BT2 also inhibited FosB (FIGS. 4D & 8C) and VCAM-1 (FIG. 4E) immunoreactivity in Matrigel plugs.
In further experiments, we compared the biological potency of BT2 with curcumin (Ye, N., et al., J Med Chem 57, 6930-6948 (2014)) in the endothelial network formation assay. While BT2 abolished network formation at 10/1 after 4 h, no inhibition was observed with curcumin at this concentration (FIG. 12). Curcumin appeared to inhibit network formation at 30 μM by ˜25% and 40 μM by ˜50% (FIG. 12), indicating BT2 is >40-fold more potent than curcumin in this assay.
BT2 structural analogues lack the biological potency of BT2. We next explored whether the biological potency or solubility of BT2 could be improved by structural modification. Dibenzoxazepinones are typically poorly soluble in water. Six BT2 analogues (aside from BT3) were generated (BT2-MeOA, BT2-EOMe, BT2-Pr, BT2-IC, BT2-MO, BT2-IMO) (Table 1). BT2-MeOA was synthesized by coupling methoxyacetic acid with 2-amino-10-ethyldibenzo[b,f][1,4] oxazepin-11 (10H)-one (BT3) while BT2-IC was synthesized using the diisobutyl dicarbonate (FIG. 6B, Scheme 1). BT2-Pr and BT2-EOMe were synthesized from commercially available (1) and (2) (FIG. 6B, Scheme 2) with the same protocol used to prepare BT2. A tri-deuterated derivation of BT2 was synthesized for stability analysis by alkylating 2-nitro-10H-dibenzo[b,f][1,4]oxazepin-11-one (3) with d₃-iodoethane followed by reduction of the nitro group to give compound (4) (FIG. 6B, Scheme 3). This intermediate was reacted with diethyl pyrocarbonate to give the desired product. Alkylation of 2-nitro-10H-dibenzo[b,f][1,4]oxazepin-11-one (3) with oxetan-3-ylmethyl methanesulfonate gave both O- and N-alkylated products in 13% and 48% yield respectively after column chromatography (FIG. 6B, Scheme 3). The O- and N-alkylated products were reduced to give the corresponding anilines (5) and (6) (FIG. 6B, Scheme 3) and then converted in the usual manner using diethyl pyrocarbonate to the target products BT2-IMO and BT2-MO.
Dilution of these compounds in medium containing serum revealed that only one these analogues (BT2-MeOA) had greater solubility than BT2 and that BT3 was the most soluble of all these dibenzoxazepinones. Adding serum to the diluent increased BT2 solubility, consistent with reports that serum albumin can increase the dissolution of unionizable drugs (Khoder, M., et al., Pharm Dev Technol 23, 732-738 (2018)). Neither BT2-MeOA nor any other BT2 analogue had the ability to inhibit serum-inducible proliferation (FIG. 5A) or network formation on Matrigel (FIG. 5B) as, or more potently than BT2. BT2-IC showed some inhibition of network formation at higher concentrations (FIG. 13B).
Since BT2 suppressed ERK phosphorylation, we hypothesized that BT2 may interact with MEK1 or MEK2. Binding of BT2 and PD98059 to recombinant His-MEK-1 or His-MEK2 was tested by surface plasmon resonance (SPR). Over the concentration range able to be assayed, BT2 bound to His-MEK1 significantly better than to His-MEK2 (FIG. 5C). In contrast, and as expected, PD98059 bound to both His-MEK1 and His-MEK2 (Dudley, D. T., et al., Proc Natl Acad Sci USA 92, 7686-7689 (1995)) (FIG. 5C). Over equivalent concentration ranges, there was no appreciable binding of BT3, BT2-MeOA or BT2-Pr to either MEK1 or MEK2. BT2-IC showed some interaction with MEK1 (but not MEK2). The drop-off in interaction at higher concentrations is possibly due to BT2-IC insolubility (limit of solubility 8±2 μM determined by ¹H 1D NMR spectroscopy). Western blotting revealed that BT2-IC inhibited both ERK phosphorylation (FIG. 13A) and network formation (FIG. 13B) at 3 μM (albeit less potently than BT2) but not at IAA (FIGS. 5B & D).
BT2 retains stability and biological potency after sonication and 100° C. treatment or autoclaving. Finally, in considering the potential pharmaceutical appeal of BT2, we explored whether this compound (as a sonicated preparation in saline containing 0.01% DMSO and 0.5% Tween 80) retained biological potency and stability after extreme heat treatment. Rapid resolution liquid chromatography/tandem mass spectrometry (RRLC-MS/MS) revealed that BT2 remains stable with or without heat treatment (100° C. for and 6 weeks storage at 22° C., with only 0.2% and 1% discrepancy in BT2 content in non-heat treated and heat-treated formulations, respectively (FIGS. 14A-B). BT2 retained its ability to inhibit serum-inducible endothelial proliferation under these conditions (FIG. 14C). Even more surprisingly, there was no loss in biological efficacy or degradation even up to 16 months (FIGS. 14D-F). Remarkably, the BT2 formulation remained stable and biologically active 4 months after standard autoclaving and storage at 22° C. (FIG. 14G). Antibodies and other proteins, which comprise all current nAMD/DME drugs, are typically inactivated by extreme heat (Jones, F. S., J Exp Med 46, 291-301 (1927).
BT2 inhibits monocytic cell adhesion to IL-1β-treated endothelium in vitro and monocytic transendothelial migration toward MCP-1 in vitro. VCAM-1 mediates monocyte adhesion in human umbilical vein endothelial cells (Gerszten, R. E., et al., Circ Res 82, 871-878 (1998)). In an in vitro model involving calcein-labeled THP-1 monocytic cells and endothelial cells pretreated with IL-β, THP-1 adhesion to endothelial cells is inhibited by BT2 (FIG. 15A). BT2 also inhibits the transendothelial migration of THP-1 monocytes toward MCP-1 from the upper chamber to the lower chamber (FIG. 15B).
Intraperitoneal administration of BT2 prevents footpad swelling, bone destruction and VCAM-1 and ICAM-1 expression in arthritic mice. Having established the in vitro anti-angiogenic and anti-inflammatory properties of BT2, we hypothesized that BT2 may be useful in a complex pro-inflammatory setting such as collagen antibody induced arthritis (Khachigian, L. M. Nature Protocols 1, 2512-2516 (2006)). Hind footpad thickness induced in this model is inhibited by a single administration of 30 mg/kg BT2 (FIGS. 16A & B). H&E staining revealed significant inflammation in CAIA mice injected is reduced by BT2 (FIG. 16C). 3D micro-CT analysis on hind limbs revealed that BT2 inhibits bone destruction (FIGS. 16D & E). In support of these findings, we made use of the fact that tartrate-resistant acid phosphatase (TRAP) activity is a key histocytochemical marker of bone-degrading osteoclasts (Ballanti, P., et al., Osteoporosis International 7, 39-43 (1997)). BT2 reduced TRAP staining in the joints (FIG. 16F). BT2 was found to inhibit VCAM-1 and ICAM-1 expression in bone (FIG. 16G). Additionally, BT2 (30 mg/kg) reduced plasma levels of IL-1β, IL-2 and IL-6 to normal levels but did not change IL-4 or IL-10.
No evidence of BT2 toxicity following intraperitoneal, intraarticular or gavage administration. BT2 (3 or 30 mg/kg) was administered to Balb/c mice by one of 3 routes (intraperitoneal injection, intraarticular injection or oral gavage) and tissues were assessed for signs of toxicity. There was no histopathological evidence of toxic damage due to BT2 (Table 2). Livers from most mice in all groups contained minimal to mild, infrequent inflammatory foci, occasionally associated with necrosis of individual hepatocytes or small groups of hepatocytes. This is likely a common, spontaneous background lesion in laboratory mice (Taylor, I. Mouse. in Background lesions in laboratory animals (ed. McInnes, E. F.) 45-75 (Saunders Elsevier, Edinburgh, 2012)) and not test item related. Livers from most mice in groups administered i.p. exhibited minimal to mild inflammation over the capsule, consistent with a non-specific peritoneal reaction to the injection and the effect being unrelated to the test item. Kidneys from one of 5 control mice and 4 of 30 BT2-treated mice contained infrequent inflammatory foci. Again this is a common spontaneous background lesion in laboratory mice and not test item related. Inflammation involving the pelvis of the kidney may have been due to ascending bacterial infection of the urinary tract. In liver and lung, there were other very infrequent, minimal changes not related to the treatment group. In summary, there was no histopathological evidence of toxicity following intraperitoneal, intraarticular injection or gavage administration of BT2.
Finally, GLP-compliant pharmacokinetics and ocular tolerance studies conducted by Iris Pharma (France), single intravitreal injection in rabbits (10 μg/50 μl BT2) revealed that BT2 is well tolerated macroscopically and histologically after 28 days with an ocular half-life (t_{1/2}) of 3.3 days.

TABLE 2
Severity grading of histopathology findings following intraarticular, intraperitoneal
or gavage delivery of BT2. Severity of lesions was graded histologically as follows:
0 = no abnormalities detected; 1 = minimal change; 2 = mild change; 3 = moderate
change; 4 = severe change; NA = not assessed. n = 5 mice per group.
IA denotes intraarticular, IP intraperitoneal. Vehicle was DMSO.

| Group | Control (Untreated) | | | | |
|---|---|---|---|---|---|
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 1 | 1 | 1 | 0 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 1 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IA Vehicle | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 0 | 1 | 1 | 1 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IA BT2 3 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 1 | 1 | 1 | 0 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 1 | 0 | 0 | 0 |
| inflammation, renal pelvis | 2 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IA BT2 30 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 1 | 1 | 1 | 1 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IP Vehicle | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 0 | 1 | 1 | 1 | 1 |
| inflammation, peritoneal | 1 | 2 | 1 | 1 | 1 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IP BT2 3 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 1 | 0 | 1 | 1 |
| inflammation, peritoneal | 0 | 0 | 2 | 1 | 1 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| osseous metaplasia | 0 | 1 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | IP BT2 30 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 1 | 1 | 1 | 2 |
| inflammation, peritoneal | 1 | 1 | 2 | 1 | 2 |
| mineralisation, subcapsular | 0 | 1 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 4 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | Gavage Vehicle | | | | |
| Animal | Mouse 1 | Mouse | 2 Mouse 3 | Mouse 4 | Mouse 5 |

Liver

| | | | | | |
|---|---|---|---|---|---|
| inflammation, parenchymal | 1 | 0 | 1 | 1 | 1 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | NA |
| Group | Gavage BT2 3 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 0 | 0 | 0 | 1 | 1 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 1 | 0 |
| inflammation, renal pelvis | 0 | 0 | 0 | 0 | 0 |

Lung

| osseous metaplasia | 0 | 0 | 0 | 0 | 0 |
|---|---|---|---|---|---|
| Spleen | 0 | 0 | 0 | 0 | 0 |
| Heart | 0 | 0 | 0 | 0 | 0 |
| Eye (left and right) | 0 | 0 | 0 | 0 | 0 |
| Group | Gavage BT2 30 mg/kg | | | | |
| Animal | Mouse 1 | Mouse 2 | Mouse 3 | Mouse 4 | Mouse 5 |

Liver

| | | | | | |
|---|---|---|---|---|---|
| inflammation, parenchymal | 1 | 0 | 0 | 1 | 0 |
| inflammation, peritoneal | 0 | 0 | 0 | 0 | 0 |
| mineralisation, subcapsular | 0 | 0 | 0 | 0 | 0 |

Kidney (left and right)

| | | | | | |
|---|---|---|---|---|---|
| inflammation, interstitial | 0 | 0 | 0 | 0 | 0 |
| inflammation, renal pelvis | 0 | 0 | 0 | 0 | 0 |

Lung

| | | | | | |
|---|---|---|---|---|---|
| osseous metaplasia | 0 | 0 | 0 | 0 | 0 |
| Spleen | 0 | 0 | 0 | 0 | 0 |
| Heart | 0 | 0 | 0 | 0 | 0 |
| Eye (left and right) | 0 | 0 | 0 | 0 | 0 |

### Discussion

New therapeutic approaches complementing existing VEGF-based strategies for nAMD/DR are needed (Apte, R. S., et al., Cell 176, 1248-1264 (2019)). While IVT anti-VEGF remains first-line therapy for retinal leakage, alternative therapies are required as many patients do not respond optimally, or the response is not sustained. The Comparison of AMD Treatments Trials (CATT) study with 647 nAMD patients treated with ranibizumab or bevacizumab showed that vision gains during the first 2 years were not maintained at 5 years (Maguire, M. G., et al., Ophthalmology 123, 1751-1761 (2016); Pedrosa, A. C., et al., Clin Ophthalmol 10, 541-546 (2016)). The AURA^{iv} study of 2227 nAMD patients in 8 European countries also revealed that while anti-VEGF therapy resulted in initial improvement in visual acuity, gains were not maintained over time and declined, mainly due to undertreatment (Holz, F. G., et al., Br J Ophthalmol 99, 220-226 (2015)).
Here we report our discovery and biological characterization of a novel dibenzoxazepinone from a high throughput screen of ˜100,000 compounds. BT2 blocks cell proliferation, migration, wound repair and network formation in vitro. This compound demonstrates efficacy in animal models of vascular leakage and angiogenesis (Carneiro, A., et al., Acta Ophthalmol 87, 517-523 (2009); Ameri, H., et al., Invest Ophthalmol Vis Sci 48, 5708-5715 (2007); Pan, C. K., et al., J Ocul Pharmacol Ther 27, 219-224 (2011)) that have served as key platforms in the development of nAMD/DR therapies used by millions today. BT2 prevented retinal vascular permeability in rats following choroidal laser injury as effectively as first-line therapy for nAMD and DME following 6 aflibercept injections compared with 2 of BT2 at the same dose. BT2 reduced CD31 staining in the IPL and INL, consistent with VEGF-A gain-of-function studies in amacrine and horizontal cells after studies that crossed Ptfla-Cre mice with floxed Vhl (Vhlf/f) mice to induce pseudohypoxia revealed massive neovascularization in the IPL and INL (Usui, Y., et al., J Clin Invest 125, 2335-2346 (2015)). In rabbits, we found that BT2 inhibited retinal vascular leakiness induced by VEGF-A₁₆₅.
While BT2 suppressed the inducible expression of VEGF-A₁₆₅, its effects in the retina were not confined to VEGF. BT2 inhibited ERK activation and VCAM-1 expression, both implicated in the pathogenesis of nAMD and DR (Kyosseva, S. V., et al., Ophthalmol Eye Dis 8, 23-30 (2016); Ye, X., et al., Invest Ophthalmol Vis Sci 53, 3481-3489 (2012); Jonas, J. B., et al., Arch Ophthalmol 128, 1281-6 (2010); Barile, G. R., et al., Curr Eye Res 19, 219-227 (1999)). Our findings suggest the existence of a pERK-FosB/ΔFosB-VCAM-1 cascade under conditions of cytokine stimulation. BT2 also inhibited a range of other genes involved in cell growth, migration, angiogenesis and inflammation. BT2 is more potent than PD98059 and >40-fold more potent than curcumin, the main active ingredient in the golden spice turmeric that inhibits AP-1 (Ye, N., et al., J Med Chem 57, 6930-6948 (2014) and is widely used for medicinal purposes despite double-blind placebo controlled clinical trials of curcumin not having been successful (Nelson, K. M., et al., J Med Chem 1620-1637 (2017)).
We synthesised BT2 analogues bearing a variety of substitutions at the 2- and 10-positions of the 2-amino-dibenzo[b,f][1,4] oxazepin-11(10H)-one ring system. Minor variations of the carbamate moiety (BT2-MeOA and BT2-IC) markedly affected activity as did modifications at the 10-position (BT2-Pr, BT2-EOMe, BT2-MO and BT2-IMO). We expected that BT2-EOMe, BT2-MO and BT2-IMO, all of which have lower calculated log Ps, would have increased water solubility. Although BT2-MeOA (and BT3) were more soluble than BT2, two separate assays revealed BT2 remained the most biologically potent of all these compounds indicating that larger substituents at the 2- and 10-positions are not advantageous. Comparison of BT2 with BT2-MeOA, which has the same molecular formula/weight and is an isomer of BT2 (linked through an amide) indicates that the carbamate moiety at the 2-position in BT2 is critical to BT2 function. BT2 may be amenable to lipid-based drug delivery systems, such as self-emulsifying delivery methodologies, that have improved oral absorption of poorly water-soluble drugs and facilitated high-dose toxicological studies (Chen, X. Q., et al., J Pharm Sci 107, 1352-1360 (2018)).
Rodent and rabbit models are useful in recreating certain features of retinal disease in humans, but may not totally recapitulate the human condition since nAMD and DR are complex, multifactorial chronic diseases that cannot be precisely recreated in acute experiments with single stimuli (Robinson, R., et al., Dis Model Mech 5, 444-456 (2012)). While rats offer advantages of rapid disease progression and comparative low cost, rats (like mice) do not possess a macula (Pennesi, M. E., et al., Mol Aspects Med 33, 487-509 (2012)). The size of the rabbit eye is more akin to the human eye but its posterior segment circulation differs from primates and rodents and rabbits also lack a macula (Chen, S., et al., Expert Rev Opthalmol 9, 285-295 (2014)). BT2 may overcome limitations in translatability that have hampered the broader use of humanized and species-specific reagents in animal models (Lu, F., et al., Graefes Arch Clin Exp Ophthalmol 247, 171-177 (2009)).
BT2 effects outside the retina. There is also a need for new and effective anti-inflammatory and anti-arthritic agents. Around one third of patients treated with a TNF inhibitor do not achieve 20% improvement based on American College of Rheumatology criteria (Klak, A., et al., Rheumatologia 54, 177-186 (2016)); Rubbert-Roth, A. & Finckh, A. Arthritis Research & Therapy 11 Suppl 1, S1 (2009)) which may relate to serum IFN-β/α ratio (Wampler Muskardin, T., et al., Annals of the Rheumatic Diseases 75, 1757-1762 (2016)). p-ERK levels are elevated in synovial tissue from RA patients compared with normal individuals (Thiel, M. J., et al., Arthritis Rheum 56, 3347-3357 (2007)). Moreover, serum sVCAM1 levels reflect the clinical status in RA (Navarro-Hernandez, R. E. et al., Disease Markers 26, 119-126 (2009)) and decrease in RA patients as the condition is relieved (Wang, L., et al., Experimental and Therapeutic Medicine 10, 1229-1233 (2015)). We found that BT2 delivered systemically in CAIA mice inhibited joint inflammation and bone erosion. BT2 also suppressed monocytic cell adhesion to endothelial cells and monocytic transendothelial migration to MCP-1 in vitro. Moreover systemic administration of BT2 in mice prevents footpad swelling, TRAP staining and bone destruction. Inflammation is also thought to drive all phases of atherosclerosis, from initiation, progression, and ultimately plaque rupture and infarction, causing further inflammation. The recent CANTOS (Hansson, G. K. Circulation 136, 1875-7 (2017); Ridker, P. M., et al. N Engl J Med 377, 1119-31 (2017)), COLCOT (Tardif, J. C., et al. N Engl J Med 381:2497-2505 (2019)) and tocilizumab (Kleveland, O., et al. Eur Heart J 37, 2406-13 (2016)) clinical trials revealed that inflammation is a treatable mechanism in cardiovascular disease. However, patients treated with existing anti-inflammatory approaches (such as canakinumab and colchicine) remain at considerable risk for major adverse cardiac events even with the widespread use of statins and antiplatelet therapies (Ridker, P. M., et al. N Engl J Med 377, 1119-31 (2017); Tardif, J. C., et al. N Engl J Med 381:2497-2505 (2019); Thompson, P. L. Clin Ther. 41, 41:8-10 (2019). There is also a paucity of clinically effective anti-inflammatory small molecule drugs for cardiovascular disease beyond statins (Collins, R. et al. Lancet 388, 2532-61 (2016)). This indicates the therapeutic potential of BT2 in RA and other inflammatory disease.
In conclusion, BT2 offers a new tool in the armamentarium targeting vascular permeability, angiogenic and inflammatory indications. BT2 served as a molecular tool to establish an ERK-FosB-VCAM1 axis mediating vascular permeability. Together with a favorable toxicological profile, our findings suggest clinical utility of this compound for retinal disease and RA. Unlike current clinically used antibody- or protein-based therapies that principally target the VEGF system, BT2 inhibits the inducible expression of multiple genes that underpin angiogenic and inflammatory processes not limited to VEGF. That BT2 retains biological potency even after boiling or autoclaving and several months' storage at room temperature adding further to its pharmaceutical appeal. Like triamcinolone acetonide, BT2 is poorly soluble in water and as such, could potentially offer a further advantage that a bolus injection can form a depot at the site of injection facilitating gradual release (Yang, Y., et al., Retina 35, 2440-2449 (2015)). Moreover, BT2 may be used in intravitreal reservoirs or implant strategies and ocular delivery systems facilitating sustained release (Kang-Mieler, J. J., et al. Eye (Lond) 34, 1371-1379 (2021)).

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1.-59. (canceled)

60. A method of reducing vascular permeability, neovascularisation, angiogenesis, inflammation, cell migration and/or proliferation in a subject, comprising administering an effective amount of a compound of formula (II), or a pharmaceutically acceptable salt thereof: 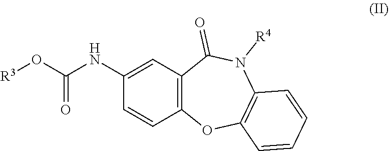
wherein:
R³ is straight or branched C₁-C₆ alkyl; and
R⁴ is straight or branched C₁-C₆ alkyl,
or R⁴ is 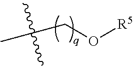
wherein q is 1, 2, 3 or 4; and R⁵ is straight or branched C₁-C₆ alkyl.

61. The method of claim 60, wherein R³ is straight C₁-C₆ alkyl or branched C₁-C₆ alkyl.

62. The method of claim 60, wherein R³ is —CH₂CH₃ or —CH₂CH(CH₃)₂.

63. The method of claim 60, wherein R⁴ is straight C₁-C₆ alkyl or branched C₁-C₆ alkyl.

64. The method of claim 60, wherein R⁴ is —CH₂CH₃ or —CH₂CH(CH₃)₂.

65. The method of claim 60, wherein R⁴ is 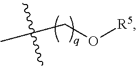
wherein q is 1, 2, 3 or 4; and R⁵ is straight C₁-C₆ alkyl or branched C₁-C₆ alkyl.

66. The method of claim 65, wherein q is 2.

67. The method of claim 65, wherein R⁵ is —CH₃.

68. The method of claim 65, wherein q is 2 and R⁵ is —CH₃.

69. The method of claim 60, wherein the compound of formula (II) is a compound of formula (II-1): 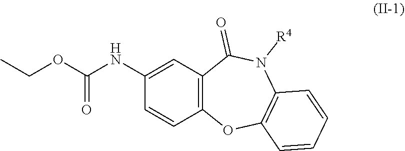
wherein:
R⁴ is straight or branched C₁-C₆ alkyl;
or R⁴ is: 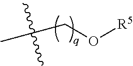
wherein q is 1, 2, 3 or 4; and R⁵ is straight or branched C₁-C₆ alkyl.

70. The method of claim 69, wherein compound of formula (II-1) is selected from:

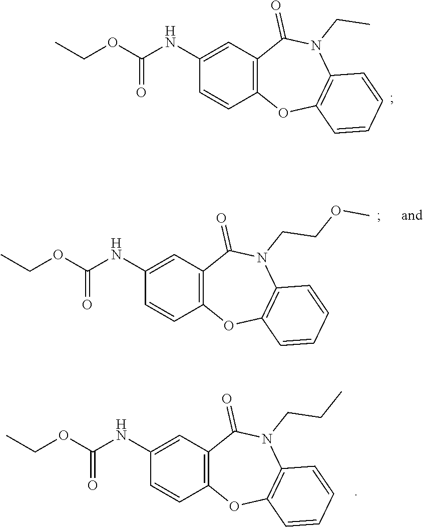

71. The method of claim 70, wherein the compound of formula (II-1) is:

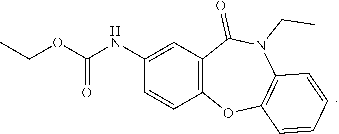 [SMILES: CCOC(=O)Nc1ccc2c(c1)C(=O)N(CC)c1ccccc1O2]

72. The method of claim 60, wherein the reducing vascular permeability, neovascularisation, angiogenesis, inflammation, cell migration and/or proliferation treats a disease or condition associated with vascular permeability, neovascularisation, angiogenesis, inflammation, cell migration and/or cell proliferation in the subject.

73. The method of claim 72, wherein the disease or condition associated with vascular permeability, neovascularisation, angiogenesis, inflammation, cell migration and/or cell proliferation is a disease or condition mediated by AP-1, and/or FosB/ΔFosB and/or ERK1/2 and/or VCAM-1 and/or VEGF-A and/or IL1-β.

74. The method of claim 72, wherein the disease or condition is selected from the group consisting of arthritis;
rheumatoid arthritis;
bone destruction;
age-related macular degeneration;
diabetic retinopathy;
macular edema;
vascular leakage;
vascular permeability;
retinal vascular permeability;
angiogenesis;
endothelial cell dysfunction;
atherosclerosis;
stroke;
myocardial infarction;
peripheral vascular disease;
stenosis;
restenosis;
inflammation;
cytokine storm;
pulmonary inflammation; and
pulmonary fibrosis.

75. A pharmaceutical composition comprising a compound of formula II, or a pharmaceutically acceptable salt thereof: 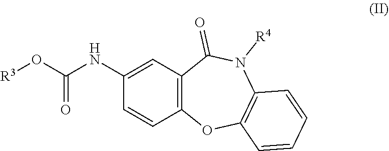
wherein:
R³ is straight or branched C₁-C₆ alkyl; and
R⁴ is straight or branched C₁-C₆ alkyl,
or R⁴ is 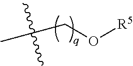
wherein q is 1, 2, 3 or 4; and R⁵ is straight or branched C₁-C₆ alkyl, and a pharmaceutically acceptable carrier.

76. The pharmaceutical composition of claim 75, wherein R³ is straight C₁-C₆ alkyl or branched C₁-C₆ alkyl.

77. The pharmaceutical composition of claim 75, wherein R³ is —CH₂CH₃ or —CH₂CH(CH₃)₂.

78. The pharmaceutical composition of claim 75, wherein R⁴ is straight C₁-C₆ alkyl or branched C₁-C₆ alkyl.

79. The pharmaceutical composition of claim 75, wherein R⁴ is —CH₂CH₃ or —CH₂CH(CH₃)₂.

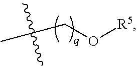

80. The pharmaceutical composition of claim 75, wherein R⁴ is wherein q is 1, 2, 3 or 4; and R⁵ is straight C₁-C₆ alkyl or branched C₁-C₆ alkyl.

81. The pharmaceutical composition of claim 80, wherein q is 2.

82. The pharmaceutical composition of claim 80, wherein R⁵ is —CH₃.

83. The pharmaceutical composition of claim 80, wherein q is 2 and R⁵ is —CH₃.

84. The pharmaceutical composition of claim 75, wherein the compound of formula (II) is a compound of formula (II-1): 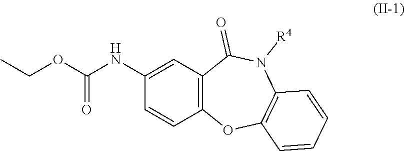
wherein:
R⁴ is straight or branched C₁-C₆ alkyl;
or R⁴ is: 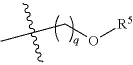
wherein q is 1, 2, 3 or 4; and R⁵ is straight or branched C₁-C₆ alkyl.

85. The pharmaceutical composition of claim 75, wherein the compound of formula (II) is:

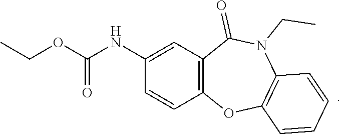 [SMILES: CCOC(=O)Nc1ccc2c(c1)C(=O)N(CC)c1ccccc1O2]

86. A pharmaceutical composition comprising a compound of the following formula: 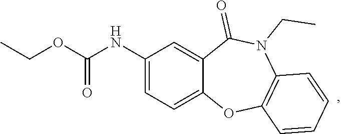 [SMILES: CCOC(=O)Nc1ccc2c(c1)C(=O)N(CC)c1ccccc1O2]
or a pharmaceutically acceptable salt thereof, and a pharmaceutically acceptable carrier.

87. The method of claim 60, wherein AP-1-dependent gene expression and/or ERK1/2-dependent gene expression and/or FosB/ΔFosB expression and/or VCAM-1 expression and/or VEGF-A expression is reduced in a cell of the subject.

88. A kit for reducing AP-1-dependent gene expression and/or ERK1/2-dependent gene expression and/or FosB/ΔFosB expression and/or VCAM-1 expression and/or VEGF-A expression, comprising a compound of formula II, or a pharmaceutically acceptable salt thereof, or a pharmaceutical composition of claim 75.

89. The kit of claim 88, wherein the compound is: 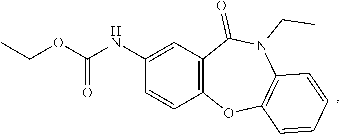 [SMILES: CCOC(=O)Nc1ccc2c(c1)C(=O)N(CC)c1ccccc1O2]
or a pharmaceutically acceptable salt thereof.
